(19) United States
(12) Patent Application Publication
(10) Pub. No.: US 2021/0310061 A1
(43) Pub. Date: Oct. 7, 2021

(54) DNA AMPLIFICATION METHOD FOR PROBE GENERATION

(71) Applicant: Dana-Farber Cancer Institute, Inc., Boston, MA (US)

(72) Inventors: Gerassimos Makrigiorgos, Chestnut Hill, MA (US); Ka Wai Leong, Melrose, MA (US); Fangyan Yu, Dorchester, MA (US)

(73) Assignee: Dana-Farber Cancer Institute, Inc., Boston, MA (US)

(21) Appl. No.: 17/268,941

(22) PCT Filed: Aug. 16, 2019
(86) PCT No.: PCT/US2019/046937

§ 371 Date: Feb. 16, 2021

Related U.S. Application Data
(60) Provisional application No. 62/765,073, filed on Aug. 17, 2018.

(51) Int. Cl. C12Q 1/6853; C12Q 1/6811

(52) U.S. Cl. CPC C12Q 1/6853; C12Q 1/6811

(57) ABSTRACT

Disclosed herein are compositions and methods for isothermal amplification of dsDNA without the use of primers. The dis-closed compositions and methods may be used for the generation of probes used in hybrid-capture techniques that usually precede sequencing. Methods of using the probes for capturing target DNA are also provided.

### GOVERNMENT SUPPORT

This invention was made with government support under grant numbers R33 CA217652 and R01 CA221874 awarded by the National Institutes of Health. The government has certain rights in the invention

### RELATED APPLICATIONS

This application is a national stage filing under 35 U.S.C. § 371 of International PCT Application PCT/US2019/046937, filed Aug. 16, 2019, which claims priority under 35 U.S.C. § 119(e) to U.S. Provisional Application, U.S.S.N. 62/765,073, filed on Aug. 17, 2018, the contents of each of which are incorporated herein by reference in their entirety.

### BACKGROUND

Sample preparation for sequencing often relies on DNA hybrid capture (Hodges et al., Nat Genet 2007, 39:1522-7; Mamanova et al., Nature Methods 2010, 7:111-8), wherein probes specific to DNA targets of interest are immobilized on solid support, and used to capture and enrich DNA targets of interest from a sample to be interrogated. Generating such immobilized probes has become very popular, however it is currently an expensive process. A mere 10-20 hybridization capture reactions can cost thousands of dollars.

### SUMMARY

Disclosed herein is a technique of isothermal DNA amplification that can be used to generate probes for hybrid capture in a way that is several orders of magnitude less expensive than currently used techniques. In some embodiments, the method of isothermally amplifying DNA uses small amounts of input dsDNA (e.g., existing probes) to generate a much higher amount of smaller versions of the input dsDNA, thereby reducing the cost of probes. The product of the disclosed amplification reaction is abundant amounts (e.g., 100-10,000 times the input DNA) of short DNA fragments which can be used for capture hybridization reactions. In addition to being used for making capture or hybridization probes, the compositions and methods disclosed herein can be used in a number of applications including, but not limited to, amplification of DNA from a biological sample so as to identify the source or species from which the DNA originated by methods such as sequencing, and amplification of clinical DNA samples (e.g., from biopsies, FFPE samples, or circulating DNA) to identify rare mutations using techniques such as nuclease-assisted mutation enrichment (NAME).

The present method is based, at least in part, on the recognition that input double-stranded DNA (dsDNA) can be amplified to produce shorter copies under isothermal conditions using a chain reaction caused by the simultaneous action of a nicking nuclease (e.g., double stranded DNA specific nuclease, DSN) and a strand-displacing polymerase (e.g., a Bst polymerase). In some embodiments, the DSN and/or the strand-displacing polymerase are thermostable. As illustrated in FIG. 1, a nicking nuclease generates non-specific, random nicks on sense and antisense input DNA strands, while a strand-displacing polymerase initiates DNA synthesis at nicked positions in the 5′ to 3′ direction by displacing the existing DNA strand. Once a sense DNA strand is displaced, it hybridizes with a similarly displaced and complementary antisense strand and re-forms double stranded DNA, thereby propagating the reaction and resulting in exponential amplification. The daughter DNA strands have smaller size than the original parent input DNA molecule and as the reaction proceeds, the size of the daughter DNA decreases progressively. This chain reaction eventually stops because of formation of by-products (e.g., diphosphate) that inhibits the action of one or both of the enzymes, or inability of the DSN to bind to daughter strands either because of their shorter length or because they are not in hybridized double-stranded form.

The final product of the reaction is short, double-stranded DNA oligonucleotides (‘probes’) corresponding in sequence to sections of the original parent DNA molecule. As compared to the original input target DNA, the product of the amplification reaction can be 100-10,000 times higher in nanograms, depending on conditions applied.

The utilization of thermostable enzymes for the current amplification reaction, such as BST and DSN, enables a highly efficient generation of amplified probes. While many isothermal DNA amplification methods are known in the art (see e.g., Gill et al., Nucleosides Nucleotides Nucleic Acids 2008, 27:224-43), they did not utilize thermostable enzymes and often require the use of one or more sets of primers, and therefore knowledge of specific sequences of the DNA to be amplified. Contrarily, the presently disclosed isothermal DNA amplification method requires no primers and thus, no specific knowledge of input DNA sequence.

In one aspect, provided herein is an isothermal DNA amplification method of generating probes from a sample of input double-stranded DNA (dsDNA). In some embodiments, the method comprises:
(a) forming a reaction mixture comprising:
   the input dsDNA,
   a nicking nuclease active at a temperature T, wherein the nicking nuclease incorporates random single-stranded breaks into dsDNA,
   a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and
(b) subjecting the reaction mixture to the temperature T under which both the nicking nuclease and the strand-displacing polymerase are active, thereby forming the probes.

In some embodiments, a method of generating probes further comprises, prior to forming the reaction mixture, forming input dsDNA from input ssDNA. In some embodiments, a method of forming input dsDNA comprises:
forming a reaction mixture comprising:
   the input ssDNA,
   deoxynucleotidyl transferase (TdT), wherein the TdT has the ability to add a polyA tail to the 3′ end of ssDNA, and
   poly-dT-primers, wherein the poly-dT primers consist of equal amounts of a poly-dT-primer with an extra G nucleotide at the 3′ end, a poly-dT-primer with an extra C nucleotide at the 3′ end, and a poly-dT-primer with an extra A nucleotide at the 3′ end;
forming a polyA tail on the 3′ end of the ssDNA;
subjecting the reaction mixture to a temperature under which the poly-dT-primers anneal to the polyA tails on the ssDNA; and
subjecting the reaction mixture to a polymerase and to a temperature under which the poly-dT-primers extend to form dsDNA.

In some embodiments, a method of forming input dsDNA comprises performing a Klenow-fragment enzymatic reaction on the input ssDNA. In some embodiments, a Klenow-fragment enzymatic reaction is performed in the presence of random oligonucleotides (e.g., random hexamers or random decamers).

In some embodiments, a method of generating probes as disclosed herein comprises forming input dsDNA from single-stranded nucleic acid by adding one or more oligonucleotides that are complementary to part (e.g., 10-150 nucleotides long) of the single-stranded nucleic acid sequences, whereby oligonucleotide extension and thus formation of dsDNA occurs via polymerase reaction. In some embodiments, a polymerase reaction occurs via a strand-displacing polymerase. In some embodiments, a polymerase reaction occurs via a strand-displacing polymerase simultaneously with probe generation.

Accordingly, in some embodiments, a method of generating probes comprises:

(a) adding to a reaction vessel, or forming a reaction mixture comprising:
input single-stranded nucleic acid (e.g., ssDNA),
one or more oligonucleotides that are complementary to at least a part of the input ssDNA, wherein the oligonucleotides are capable of extending in the presence of a strand-displacing polymerase to form dsDNA,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and

(b) subjecting the reaction mixture to the temperature T under which both the nicking nuclease and the strand-displacing polymerase are active, thereby forming the probes.

In some embodiments, ssDNA is formed by denaturing dsDNA and used as input along with one or more oligonucleotides that are complementary to part (e.g., 10-150 nucleotides long) of the single-stranded nucleic acid sequences, so that probes formed by any one of the methods disclosed herein for generating probes have sequences specific to the oligonucleotides added.

Accordingly, any one of the methods for generating probes from a sample of input dsDNA comprises:

(a) adding to a reaction vessel, or forming a reaction mixture comprising:
the input dsDNA,
oligonucleotides that are complementary to at least a part of one or both strands of the input dsDNA,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and

(b) subjecting the reaction mixture to the temperature T under which both the nicking nuclease and the strand-displacing polymerase are active, thereby forming the probes.

In some embodiments, dsDNA is first denatured to form ssDNA before forming a reaction mixture.

In some embodiments, a method of generating probes involves use of single-stranded nucleic acid (e.g., ssDNA). In some embodiments, a method of generating probes involves use of both dsDNA and single-stranded nucleic acid (e.g., ssDNA).

In some embodiments, a method of generating probes comprises:

(a) adding to a reaction vessel, or forming a reaction mixture comprising:
input dsDNA and/or single-stranded nucleic acid (e.g., ssDNA),
oligonucleotides that are complementary to at least a part of one or both strands of the input dsDNA or single-stranded nucleic acid,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and

(b) subjecting the reaction mixture to the temperature T under which both the nicking nuclease and the strand-displacing polymerase are active, thereby forming the probes.

In some embodiments, dsDNA is first denatured to form ssDNA before forming a reaction mixture with oligonucleotides, nicking nuclease, and strand-displacing nuclease.

In some embodiments, a method of generating probes using an isothermal DNA amplification method further comprises synthesizing the input dsDNA as complementary DNA (cDNA) from RNA.

In some embodiments, probes as generated in any one of the isothermal DNA amplification methods disclosed herein are a collection of dsDNA, wherein each dsDNA is a portion of the input dsDNA having corresponding or shared sequence with the input DNA, and wherein the collection of dsDNA randomly provides a coverage of 100×-10,000× of the input dsDNA.

In some embodiments, the average length of generated probes is 15-120, 20-50, 30-80, 40-120, or 15-70 bp. In some embodiments, more than 50%, 60%, 70%, 80% and even 90% of the probes are between 15-120 or 15-70 (inclusive) bp in length. In some embodiments, the amount in nanograms of generated probes is 100-10,000 times higher than the amount of input dsDNA.

In some embodiments of any one of the isothermal amplification methods disclosed herein, a reaction mixture is subjected to a temperature T for a time period of less than ten minutes. In some embodiments of any one of the isothermal amplification methods disclosed herein, a reaction mixture is subjected to a temperature T for a time period of 4-5 minutes.

Some embodiments of any one of the isothermal dsDNA amplification methods disclosed herein further comprises inactivating the nicking nuclease and the strand-displacing polymerase.

In some embodiments, an isothermal dsDNA amplification method as disclosed herein further comprises separating the nicking nuclease and the strand-displacing polymerase from the reaction mixture. In some embodiments, a method comprises separating the generated probes from the reaction mixture.

In some embodiments, a nicking nuclease is selected from the group consisting of: double-stranded DNA specific nuclease (DSN), Shrimp-based double strand specific nucleases (dsDNase), HL-dsDNAse, and DNAse I.

In some embodiments, a strand-displacing polymerase is selected from the group consisting of: a Bst DNA polymerase, phi29 polymerase, and Klenow fragment of DNA polymerase I.

In some embodiments, a nicking nuclease is selected from the group consisting of: double-stranded DNA specific nuclease (DSN), Shrimp-based double strand specific nucleases (dsDNase), and HL-dsDNAse. In some embodiments, the nicking nuclease is present in the reaction mixture at a concentration of 0.1-0.3 units. In some embodiments, a strand-displacing polymerase is a Bst DNA polymerase. In some embodiments, the strand-displacing polymerase is present at a concentration of 6-10 units; In some embodiments the temperature T is 30-70° C.

In some embodiments, a nicking nuclease is DNAse I, and a strand-displacing polymerase is Klenow fragment of DNA polymerase I; and the temperature T is 25-45° C.

In some embodiments, dNTPs comprise one or more of biotin-dUTP, 2,6 di-amino-purinetriphosphate, and d-iosinetriphosphate, including any combination thereof.

It may be useful to validate the probes generated by any one of the isothermal DNA amplification methods disclosed herein. In some embodiments, validating generated probes comprises:

attaching the probes to a solid surface;

incubating the probes with a sample of target DNA fragments to allow hybridization of the probes and target DNA sequences, wherein each target DNA fragment is ligated to an adapter;

washing away unbound DNA fragments;

releasing the target DNA fragments that are hybridized to the probes;

amplifying the released target DNA fragments using primers that are complementary to the adapters;

amplifying the released target DNA fragments using target-specific primers; and

sequencing the amplified released target DNA fragments to determine whether the amplified released target DNA fragments are specific to the probes.

As used herein, an “adapter” is a single stranded or double-stranded oligonucleotide that can be ligated to the ends of other nucleic acid molecules (e.g., DNA or RNA). An adapter may have two blunt ends, or one blunt end and one end with an overhang. Primers that are specific to adapter can then be used to run sequencing reactions.

In some aspects, provided herein is a method of interrogating target DNA regions in a sample of DNA. In some embodiments, a method of interrogating target DNA regions in a sample of DNA comprises:

(a) providing one or more input probes, wherein each input probe is a dsDNA, each single strand of which is complementary to a target DNA region, wherein the target DNA region for each input probe is different from the target DNA region for all other input probes;

(b) generating probes comprising any one of the isothermal DNA amplification methods disclosed herein, which may comprise:
(i) forming a reaction mixture comprising:
the input probe,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and
(ii) subjecting the reaction mixture to the temperature T under which both the nicking nuclease and the strand-displacing polymerase are active, thereby forming the probes;

(c) incubating a first aliquot of the generated probes with a sample of DNA suspected to have one or more of the target DNA regions to allow the generated probes to capture complementary target DNA regions;

(d) releasing the captured complementary target DNA regions from the generated probes;

(e) amplifying the captured target DNA regions; and

(f) interrogating (e.g., by sequencing, real-time PCR, digital PCR, or other downstream assays) the captured target DNA sequences.

In some embodiments, a method of interrogating target DNA regions in a sample of DNA further comprises forming the input dsDNA probes from input ssDNA probes, or from RNA.

In some embodiments, forming input dsDNA probes comprises:
forming a reaction mixture comprising:
   one or more input ssDNA probes,
   deoxynucleotidyl transferase (TdT), wherein the TdT has the ability to add a polyA tail to the 3′ end of ssDNA,
   poly-dT-primers, wherein the poly-dT primers consist of equal amounts of a poly-dT-primer with an extra G nucleotide at the 3′ end, a poly-dT-primer with an extra C nucleotide at the 3′ end, and a poly-dT-primer with an extra A nucleotide at the 3′ end; and
forming a polyA tail on the 3′ end of the ssDNA probes;
subjecting the reaction mixture to a temperature that permits annealing of the poly-dT-primers to the polyA tails on the ssDNA probes; and
extending the poly-dT-primers to form dsDNA probes.

In some embodiments, a method of interrogating a sample of DNA further comprises, prior to performing sequencing:

incubating the amplified capture target DNA regions with a second aliquot of the generated probes to allow the probes in the second aliquot of generated probes to capture complementary target DNA regions, wherein the second aliquot of generated probes is the same or different from the first aliquot of generated probes; releasing captured complementary target DNA regions from probes of the second aliquot of generated probes; and

amplifying the released target DNA regions of the second aliquot of generated probes.

In some embodiments, a sample of DNA comprises genomic DNA obtained from a biological sample. In some embodiments, a sample of DNA comprises genomic DNA that is of a micro-organism, and the method is used to identify the species of the micro-organism.

In some embodiments, genomic DNA is from a subject suspected of having one or more mutations in one or more target regions.

In some embodiments, a method to identify a microorganism as described above further comprises performing end repair to each of the generated probes to form blunt ends, and ligating the repaired ends of the generated probes to sequencing primers.

In some embodiments, a biological sample is blood, serum, plasma, urine, cheek swab, a tissue biopsy, a bronchial lavage, or a pulmonary brushing.

Some embodiments of any one of the isothermal DNA amplification methods disclosed herein further comprises forming droplets of the reaction mixture prior to subjecting the reaction mixture to a temperature T.

Provided herein is a composition comprising a collection of dsDNA probes. In some embodiments, each probe in a composition has a sequence that corresponds to or is shared with sequence of input dsDNA. In some embodiments, a collection of probes randomly provides a coverage of 100×-1000× of input dsDNA. In some embodiments, the average length of each probe in the collection of probes is 15-120, 20-50, 30-80, 40-120 or 15-70 bp. In some embodiments, more than 50%, 60%, 70%, 80% and even 90% of the probes are between 15-120 or 15-70 (inclusive) bp in length.

Provided herein is a reaction mixture comprising:
dsDNA,
a nicking nuclease, wherein the nicking nuclease at a temperature T incorporates random single-stranded breaks into dsDNA,
a strand-displacing polymerase, wherein the strand-displacing polymerase at the temperature T recognizes a single-stranded break in dsDNA and in the presence of nucleotide triphosphates extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP).

In some embodiments, a nicking nuclease is selected from the group consisting of: double-stranded DNA specific nuclease (DSN), Shrimp-based double strand specific nucleases (dsDNase), HL-dsDNAse, and DNAse I.

In some embodiments, a strand-displacing polymerase is selected from the group consisting of: Bst DNA polymerase, phi29 polymerase, and Klenow fragment of DNA polymerase I.

In some embodiments, a reaction mixture as provided herein further comprises Mg²⁺.

In some embodiments, a reaction mixture comprises one or more of the following dNTPs: dATP, dGTP, dCTP, dTTP, and analogs thereof.

In some embodiments, the nicking nuclease in a reaction mixture is selected from the group consisting of: double-stranded DNA specific nuclease (DSN), Shrimp-based double strand specific nucleases (dsDNase), and HL-dsDNAse; and the strand-displacing polymerase is a Bst DNA polymerase. In some embodiments, such a reaction mixture is at a temperature of 44-56° C.

In some embodiments, the nicking nuclease in a reaction mixture as provided herein is DNAse I, and the strand-displacing polymerase is Klenow fragment of DNA polymerase I. In some embodiments, such a reaction mixture is at a temperature of 34-40° C.

Provided herein is a kit comprising:
a nicking nuclease, wherein the nicking nuclease at a temperature T incorporates random single-stranded breaks into dsDNA,
a strand-displacing polymerase, wherein the strand-displacing polymerase at the temperature T recognizes a single-stranded break in dsDNA and in the presence of nucleotide triphosphates extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP).

In some embodiments, a kit further comprises biotin-dUTP, 2,6 di-amino-purine, and/or d-iosinetriphosphate.

In some embodiments, a kit comprises oligonucleotides that can be used together with input dsDNA or input ssDNA to make targeted probes. These probes are complementary to at least a part of the input ssDNA or strands of the input dsDNA such that they can extend in the presence of a polymerase (e.g., a strand displacing polymerase).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows the principle of the disclosed isothermal DNA amplification chain reaction involving DSN and Bst. When dsDNA is used as input in a BST-DSN reaction, the nuclease DSN nicks one strand of dsDNA to create a recognition site for BST polymerase which then synthesizes a complement of the opposite DNA strand while displacing the parent strand. The displaced-sense (or anti-sense) DNA strands subsequently can re-hybridize to complementary strands and form daughter dsDNA. Subsequent DSN nicking and BST amplification generated an exponential amplification of daughter dsDNA while progressively reducing the resulting DNA size. When single stranded nucleic acid (e.g., ssDNA) are used as input in BST-DSN reaction, the ssDNA is first subjected to a TdT reaction in the presence of dATP to generate a poly-A tail on the 3′ end. The unpurified TdT product is then used as input in a BST-DSN reaction in the presence of an anchored-oligo-dT which is extended by BST to create dsDNA as a first step in the reaction.

FIG. 2 illustrates a work flow for using the B-dUTP probes generated by Bst-DSN to capture targeted DNA from genomic DNA or ligation-mediated PCR (LMPCR) products.

FIGS. 3A-3E illustrate the amplification of PCR products or ultramers and labeling with B-dUTP by Bst-DSN. As shown in FIGS. 3A-3C, Bst-DSN chain reaction was used to amplify 10 input PCR products (FIG. 3A), the same PCR products in the presence of biotinylated dUTP, B-dUTP (FIG. 3B) and the commercially supplied single stranded ultramers (obtained from IDT) (FIG. 3C) and labeled with B-dUTP after TdT reaction. FIG. 3D shows the products generated by Bst-DSN were analyzed by Agilent DNA Chips that show the range of molecular weight of the probes obtained, 15-100 bp depending on conditions. FIG. 3E illustrates the amount of probes generated by Bst-DSN after purification via Nucleotide removal kit.

FIGS. 4A-4C illustrates use of a mix of 10 PCR products as starting material to capture the corresponding targets from ligation-mediated PCR-amplified whole genomic DNA. FIG. 4A shows that the first round of capture has been done by using the probes generated from 10 PCR products mixture in Bst-DSN with B-dUTP. All 10 targets were validated by Two-step PCR after capture. Only the sample with on target probes and hybridized with LMPCR products (boxes) were able to capture 10 specific targets. The off target-NGLY1 were not able to be amplified by all samples. FIG. 4B shows the experimental design and results of second round of capture followed by LMPCR on 1st captured product. Different amounts of probes(500 ng/50 ng/10 ng) used in capture and different amounts of PCR products (60 ng/10 ng) as input in Bst-DSN reactions were examined. FIG. 4C shows that two specific targets, NOP14 and ZPLD1, have been successfully captured by different amount of probes and amplified by two-step PCR. In contrast, very little off-target NGLY1 DNA is captured.

FIGS. 5A-5B illustrate that use of probes made from Ultramers as starting materials captures the correct DNA target. FIG. 5A shows the experimental design for examining different amounts of ‘Ultramers’ (120-bp oligonucleotides supplied by IDT; 30 ng/10 ng/2 ng) used as input in Bst-DSN reactions, and different incubation times (4 hr/16 hr/48 hr) for hybridization capture. FIG. 5B shows that all input amounts of Ultramers used in Bst-DSN and all incubation time used in hybridization were able to capture the correct target, NOP14. In contrast, the non-specific target, ZPLD1, was not significantly captured.

FIG. 6 shows a workflow for comparing capture-based target enrichment by Bst-DSN-generated probes and capture-based target enrichment using commercial Ultramer probes from IDT. 10 ng of 120 bp biotinylated Ultramers synthesized by IDT for 33 DNA targets were either used directly for a capture reaction using the IDT protocol (bottom workflow) or used to synthesize Bst-DSN biotinylated probes followed by a double capture reaction. The probes were used to capture 33 targets from LMPCR product generated from circulating DNA (cfDNA). Miseq sequencing of the captured regions was then performed to compare the established approach (IDT probes) with the current invention (Bst-DSN probes).

FIG. 7 shows real time PCR based verification that hybrid capture using probes generated using Bst-DSN reactions isolated two of the targeted genomic regions, NEB and MAP 10. Probes generated using Bst-DSN reactions captured all on-targets (4 out of 33 tested).

FIG. 8 shows real time PCR based verification that hybrid capture using probes generated using Bst-DSN reactions isolated two of the targeted genomic regions, THSD4 and MIER3.

FIG. 9 shows real time PCR based verification that hybrid capture using probes generated using Bst-DSN reactions did not isolate two of the non-targeted genomic regions ZPLD1 and ARHGEF12.

FIG. 10 shows a comparison of sequencing reads obtained by Miseq sequencing of a LMPCR product using either Bst-DSN-generated probes (MM47) or the originally synthesized Ultramer probes from IDT (MM43). Similar coverage is obtained in the two cases, indicating the utility of Bst-DSN generated probes.

FIG. 11 illustrates Bst-DSN chain reaction performed in small compartments (droplets, emulsion, microfluidics) to increase the rate of hybridization of two daughter strands by increasing special proximity.

FIG. 12 shows target-specific capture using BST-DSN probes pre-attached to streptavidin beads. Biotinylated BST-DSN probes were first immobilized on streptavidin beads. Hybridization of LMPCR products at 65 C/60 C for 16 hours was performed with labeled beads, followed by washing steps. Captured DNA was released from beads by heating at 98 C for 2 min and PCR was performed to validate the target-specific capture.

FIG. 13 shows a workflow applied for hybridization capture prior to MiSeq sequencing. The biotin-labeled commercial oligonucleotide probes from Panels A, B, and C were used as starting material for TBD reaction using B-dUTP to generate biotin-labeled TBD probes. These TBD probes were then used to capture specific targets from LMPCR product on streptavidin beads. Following LMPCR of the bead-captured DNA, the product was either sequenced directly or subjected to a second capture and then sequenced. For comparison to TBD-probe based capture, the original biotin-labeled commercial probes were tested in the same protocol, using a single round of capture was performed by original probes and followed by LMPCR amplification. The comparison of on-target captured ability between TBD probes and original probes was then validated by MiSeq sequencing.

FIGS. 14A-14B show amplification of dsDNA with and without B-dUTP labeling in BST-DSN reaction. FIG. 14A shows amplification of dsDNA when PCR product (p53 exon 8) was used as input in BST-DSN reaction using native nucleotides (dNTPs). After amplification, BST-DSN products were purified and the product size was analyzed via electrophoresis on an Agilent Bioanalyzer. Under the conditions applied, most BST-DSN products were between 20 and 80 bp while the full range of products was 15-150 bp. FIG. 14B shows amplification of dsDNA when B-dUTP was included in the dNTPs to generate biotin-labeled DNA fragments. A similar DNA fragment size distribution as in FIG. 14A was observed.

FIGS. 15A-15D show amplification of dsDNA via BST-DSN reaction with concomitant B-dUTP labeling (FIG. 15A) 10 ng, (FIG. 15B) 30 ng, (FIG. 15C) 60 ng or (FIG. 15D) 200 ng using a 10 PCR product mix was used as total DNA input in BST-DSN reaction with B-dUTP. Following amplification, BST-DSN product size was analyzed via electrophoresis on an Agilent Bioanalyzer. Under the conditions applied, most BST-DSN products were between 20 and 80 bp while the full range of products was 15-150 bp.

FIGS. 16A-16C show amplification of Panel A, B, and C by TdT-BST-DSN (TBD) reaction with concomitant B-dUTP labeling. FIG. 16A shows a workflow used for generating TBD probes from long oligonucleotides in Panels A, B, or C. FIG. 16B shows size of B-dUTP labeled TBD products as examined by DNA electrophoresis on an Agilent Bioanalyzer. Product sizes were approximately 20-120 bp. FIG. 16B shows amount of TBD probes generated from original probes of Panel A, B, and C.

FIG. 17 shows and example of using PCR to validate the target-specific capture from B-dUTP labeled BST-DSN probes generated from a mix of 10 PCR products. 800 ng of BST-DSN probes generated from a mix of 10 PCR products covering biologically relevant DNA targets were applied for capturing DNA from LMPCR products, followed by amplification of the captured DNA. The specificity of capture was verified by target-specific PCR and melting analysis for the 10 specific targets, as compared to off-target PCR applied for randomly chosen targets. All ten targets were specifically amplified from captured DNA, while no amplification from off-target DNA was observed, based on melting curve analysis.

FIGS. 18A-18C show examination of mutant and WT target capture using BST-DSN probes. BST-DSN probes generated from WT NOP14 PCR product were applied for capturing mutated and WT alleles from cancer patient 295 LMPCR product and HMC LMPCR product, following by amplification of the captured DNA. Sanger sequencing was used to verify the mutation allelic frequency MAF of the captured DNA. FIG. 18A shows detection of 81% mutant allele (G) is prior to capture from cancer patient DNA (295). FIG. 18B shows detection of 71% mutant allele (G) following capture; FIG. 18C shows detection of 100% WT allele (C) is using capture of the same WT NOP14 PCR product hybridized with HMC LMPCR library.

FIGS. 19A-19C show comparison of target capture using BST-DSN probes vs. Panel A commercial capture probes (33) ultramer biotinylated oligonucleotides. FIG. 19A shows that 1st round capture using BST-DSN probes shows inferior on-target percentage (15-20%) as compared to capture using the original probes (40%). A 2nd round capture resulted to similar on-target percentage with a single capture using commercial probes, irrespective of capture probe input, 10-100 ng (0.31-3.12 nM). FIG. 19B shows that compared to the 1st capture using commercial probes, the 2nd round of capture of BD probes shows comparable coverage and FIG. 19C shows uniformity with 2nd round capture of BD probes.

FIGS. 20A-20B show correlation between captured DNA coverage and GC content (FIG. 20A) and free energy (FIG. 20B), panel A capture probes. A Similar correlation was found between coverage and GC content/secondary structure and free energy for both TBD probes and original ultramers. Kinefold were applied to analyze the free energy. Statistical analyses were performed with PRISM 6 software (GraphPad).

FIG. 21 shows DSN digestion points on DNA during a BST-DSN reaction. DSN cutting sites were inferred from MiSeq sequencing data, by examining the starting position of individual sequencing reads on four representative regions. The sequence positions noted in red and highlighted in yellow represent DSN cutting sites on four representative regions of TBD probes generated from Panel A. The arrows indicate the positions where an alternative enzyme (CviPII ‘nickase’) would be expected to digest the same sequences.

FIGS. 22A-22E show evaluation of target capture TBD probes generated from Panel B (190 targets) and Panel C (7,816 targets). FIG. 22A shows that compared to a single round of capture using the original, commercially available probes, using a single round of capture via TBD probes generates inferior on-target ratio (Panel B probes, 1-50 ng, 0.02-1.19 nM). Two rounds of TBD probe capture generate a superior on-target ratio, provided at least 5 ng TBD probes are used as input. FIG. 22B shows a similar conclusion as for the on-target ratio applies also to the panel B coverage. FIG. 22C shows that compared to a single round of capture using the original, commercially available probes, using a single round of capture via TBD probes generates inferior on-target ratio (Panel C probes, 1-100 ng, 0.58-58 pM). Two rounds of TBD probe capture generate a superior on-target ratio, provided at least 5 ng TBD probes are used as input. FIG. 22D shows a similar conclusion as for the on-target ratio applies also to the panel C coverage. FIG. 22E shows the fold-80 base penalty, a measure of uniformity, is presented for probe panels B and C.

FIGS. 23A-23C demonstrates TBD efficiency and resource savings. FIG. 23A shows number of double-capture reactions that can be performed following TBD generated probes, starting from oligonucleotide probes currently used for either one capture or for 16 capture reactions. FIG. 23B shows the cost of target enrichment prior to NGS using the original probes of Panel C was compared to the double-capture using 5 ng of TBD probes per round of capture. FIG. 23C shows the cost of target enrichment prior to NGS using the original probes of Panel C was compared to the double-capture using 50 ng of TBD probes per round of capture. TBD probes largely reduce the cost of target enrichment prior to NGS sequencing for either 16 or for 96 capture reactions. Commercial list prices were used for this comparison.

FIGS. 24A-C illustrate use of oligonucleotides to made dsDNA from ssDNA. single stranded target DNA (NOP14 gene sequence) and the oligonucleotides F1 and/or R1 that were used to convert the NOP14 target to double stranded DNA and to subsequently generate amplified probes containing sequence from NOP14 target DNA. FIG. 24A provides sequences of the ssNOP sequence and oligonucleotides used to make dsNOP sequence. FIG. 24B the reaction conditions for generating probes. FIG. 24C shows monitoring of the amplification of NOP14 probes in real time using the DNA intercalating dye LCgreen in the reaction. Detection of the fluorescence was performed in a real time PCR machine. Each ‘cycle’ corresponds to 12 sec of isothermal incubation at 60° C.

### DETAILED DESCRIPTION

With the rapidly decreasing cost of sequencing, methods for efficient and low cost sample preparation become increasingly important. Target enrichment prior to targeted re-sequencing comprises a major part of the effort and cost involved in sample preparation ( Mamanova et al., Nat Methods, 7, 111-118; Mertes et al., Brief Funct Genomics, 10, 374-386). Target enrichment via hybridization capture is adopted commonly, especially when large panels of DNA targets consisting of hundreds or thousands of targets need be sequenced. Such hybrid capture relies on the availability of biotinylated probe panels, which comprise a significant portion in the overall cost of sample preparation for sequencing. Approaches employing biotinylated PCR products as capture probes for small numbers of DNA targets have also been described (Maricic et al., PLoS One, 5, e14004).

The present disclosure provides a method to amplify any available panel of probes without requiring information on the sequences involved.

DNA amplification methodologies are a key feature of molecular biology and recombinant DNA technologies. They are used in many applications, for example, to identify a microorganism (e.g., a virus, or bacteria) in a biological sample, to identify mutations in a sample of genomic DNA, or building nucleic acid-based circuits to achieve a particular function in a cell. Polymerase chain reaction (PCR) is the most widely used DNA amplification method. While PCR is relatively easy, it still requires a thermocycling machine. To circumvent the need to cycle temperatures, various isothermal DNA amplification techniques (e.g., transcription mediated amplification, nucleic acid sequence-based amplification, signal mediated amplification of RNA technology, strand displacement amplification, rolling circle amplification, loop-mediated isothermal amplification of DNA, isothermal multiple displacement amplification, helicase-dependent amplification, single primer isothermal amplification, and circular helicase-dependent amplification) have been developed. These techniques require the use of one or more primer pairs, and thus, knowledge of the DNA sequence to be amplified so that primers can be designed.

Disclosed herein is a method of amplifying DNA (e.g., ssDNA or dsDNA) under isothermal conditions that does not require use of primers, or knowledge of the DNA sequence to be amplified. In some embodiments, the amplified product of this isothermal DNA amplification method is a collection of dsDNA. Accordingly, in some embodiments, disclosed herein is a method of generating probes from a sample of input double-stranded DNA (dsDNA). Also disclosed herein is a method of using the probes as generated by any one of the methods disclosed herein to capture targets from a sample of DNA suspected to have one or more of the target DNA regions. Methods involving capture of nucleic acid can be extremely costly to perform, and the biggest cost of such methods is attributed to the cost of capture probes. In some embodiments, the methods of generating probes as described herein are a means of reducing the cost of capture probes.

In some embodiments, a method of generating probes comprises forming a reaction mixture comprising a nicking nuclease and a strand-displacing polymerase, and then subjecting the reaction mixture to a temperature under which both the nicking nuclease and the strand-displacing polymerase are active, thereby forming the probes.

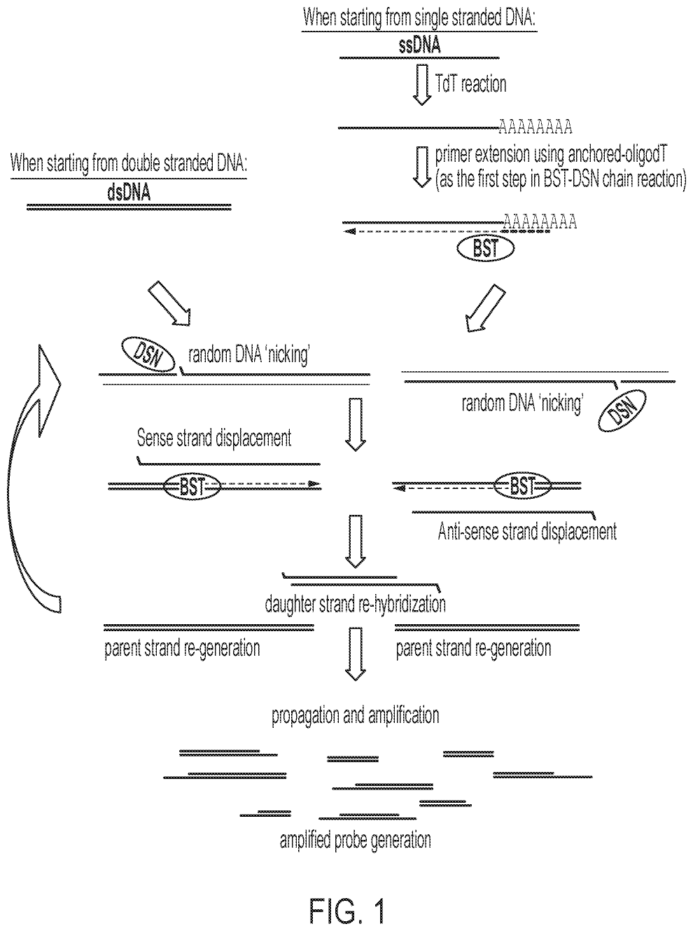

FIG. 1 provides an explanation of the chain reaction that occurs when both enzymes: a nicking nuclease, and a strand-displacing polymerase, are active and in the same mixture as input dsDNA. A nicking nuclease that is active at a temperature T incorporates random single-stranded breaks (or nicks) into the dsDNA. Simultaneously, a strand-displacing polymerase that is also active at the temperature T recognizes a single-stranded break (or “nick”) in dsDNA, and, in the presence of deoxynucleotide triphosphate (dNTPs), extends the single strand having the break, and displaces the ssDNA fragment that is 3′ relative to the break. The displaced ssDNA subsequently hybridizes with other complementary ssDNA to form daughter dsDNA, which in turn is acted upon by the two enzymes. This chain reaction results in the formation of dsDNA probes that are shorter versions of the input DNA, or short dsDNA that have sequences that are common with or shared by the input dsDNA.

The chain reaction stops after a particular time because either the daughter dsDNA on average is too short for the nicking nuclease to bind to it, or because byproducts (e.g., diphosphates) formed during the reaction inhibit the activity of either one or both of the nicking nuclease and strand-displacing polymerase. In some embodiments, the chain reaction stops because the temperature at which the reaction is carried out is too high for short daughter ssDNA to hybridize to other complementary daughter ssDNA. The rate of reaction and timing of saturation can be controlled by adjusting the concentrations of the nicking nuclease and the strand-displacing polymerase, as well as the temperature of the reaction.

### Nicking Nucleases and Strand-Displacing Polymerases

As used herein, a “nicking nuclease” is an enzyme that, when active, incorporates random “nicks,” or single-stranded breaks in dsDNA. A nicking nuclease shows strong preference for cleaving dsDNA compared to ssDNA or dsRNA. Nicking nucleases may show some activity towards ssDNA when concentrations of both the nicking nuclease and the substrate are present at high concentrations. However, this non-specific activity towards ssDNA is not detectable in the presence of competitive dsDNA. Nicking nucleases typically are able to act on dsDNA that are 10 bp or longer.

Non-limiting examples of nicking nucleases are double stranded DNA specific nuclease (DSN), Shrimp-based double strand specific nucleases dsDNase, and HL-dsDNase.

DSN is an enzyme purified from hepatopancreas of Red King (Kamchatka) crab, and is also referred to as duplex-specific nuclease. It is commercially available (e.g., from Evrogen; Catalog numbers EA001, EA002, and EA003). DSN acquires its enzymatic activity in the presence of divalent cations (e.g., Mn²⁺, Co²⁺, or Mg²⁺) at a concentration of at least 5 mM (e.g., at least 5 mM, at least 6 mM, at least 7 mM, at least 8 mM, at least 9 mM, at least 10 mM, or at least 20 mM). In some embodiments, the concentration of divalent cations in the reaction mixture of any one of the methods disclosed herein when using DSN as a nicking nuclease is 2-50 mM (e.g., 2-10 mM, 5-10 MM, 5-20 mM, 5-40 mM, 5-50 mM, 10-20 mM, or 20-50 mM).

The optimum temperature for DSN activity is 60° C. DSN retains only 10% of its maximal activity above 80° C. This sharp decrease in activity may be attributable, at least in part, to dsDNA substrate denaturation. The optimal pH for DSN activity is 6.6. At pH values between 3 and 5, DSN displays only 10% of its maximal activity. DSN is stable at a wide range of pH (from 4 to 12) and temperatures below 65° C. About 60% of DSN activity remains after 30 minutes incubation at 70° C., and 40% after incubation at 80° C.

DSN is inhibited by certain chelating agents (e.g., EDTA). Incubation of DSN with aggressive chemicals like 1% SDS, 10 mM β-mercaptoethanol, and 0.3% hydrogen peroxide at 37° C. results in only a moderate drop in activity, after 30 min incubation about 90% of activity is preserved. However, a sharp decrease in activity is observed upon chemical treatment at 60° C. SDS completely inhibits DSN activity, while β-mercaptoethanol and hydrogen peroxide induce approximately 70% and 80% loss in activity, respectively. DSN is highly sensitive to ionic force (e.g., a 10 times decrease in catalytic activity is observed in the presence of 0.2 M NaCl). The addition of chaotropic agents or polyamines to the reaction mixture also results in suppression of enzyme activity. DSN is tolerant to proteinase K treatment (incubation at 37° C. for 30 min).

Double strand specific nucleases dsDNase and HL-dsDNase are also commercially available (e.g., from ArticZymes). dsDNase is highly active in a temperature range of 20-40° C. It needs at least 2.5 mM (e.g., at least 2.5 mM, at least 5 mM, at least 7.5 mM, or at least 10 mM) of a divalent cation (e.g., Mn²⁺, Co²⁺, or Mg²⁺) for activity and has an optimal pH at 7.5. dsDNase can be heat inactivated by heat treatment at 15 min at 65° C., or 20 min at 60° C. The enzyme requires at least 1 mM DTT (e.g., 1 mM, 2 mM, 3mM or more) and pH≥8 for complete inactivation.

HL-dsDNase is active in a temperature range of 20-40° C. It needs at least 2.5 mM (e.g., at least 2.5 mM, 5 mM, 7.5 mM, or 10 mM) of a divalent cation (e.g., Mn²⁺, Co²⁺, or Mg²⁺) for activity and has an optimal pH at 7.5. HL-dsDNase can be heat inactivated by heat treatment at 5 min at 58° C. The enzyme requires at least 1 mM DTT (e.g., 1 mM, 2 mM, 3mM or more) and pH≥8 for complete inactivation.

In some embodiments, DNAse I is used as a nicking nuclease. DNAse I is an endonuclease that can digest both single- and double-stranded DNA, and is commercially available (e.g., from ThermoFisher, and New England Biolabs (NEB)). The enzyme activity is strictly dependent on Ca²⁺ and is activated by Mg²⁺ or Mn²⁺ ions. However, in the presence of Mg²⁺, DNase I cleaves each strand of dsDNA independently in a statistically random fashion. In the presence of Mn²⁺, the enzyme cleaves both DNA strands at approximately the same site, producing DNA fragments with blunt-ends or with overhang termini of one or two nucleotides. DNAse I is usually used at 37° C. where its activity is maximum. DNAse can be inactivated by physical denaturation (e.g., by mixing vigorously or vortexing).

As used herein, a “strand-displacing polymerase” is a polymerase that recognizes a nick or single-stranded break in dsDNA, and, in the presence of deoxynucleotide triphosphate (dNTPs), extends the single strand having the break, and displaces the ssDNA fragment that is 3′ relative to the break. The term “strand displacement” describes the ability to displace downstream DNA encountered during synthesis.

Non-limiting examples of strand-displacing polymerases are phi29 (e.g., available at NEB, catalog No. M0269), Bst DNA polymerase and variants thereof (e.g., wildtype Bst, Bst large fragment, Bst 2.0, and Bst 3.0 available at NEB), and Klenow fragment of DNA polymerase I.

The activity of phi29 is maximum at a temperature of 20-37° C. (e.g., 30° C.).

Bst polymerases are typically most active in the temperature range of 60-72° C. (e.g., 65° C.). Depending on the specific Bst polymerase, inactivation can be achieved by incubation at a temperature of 80° C. or higher for approximately 5 minutes.

Klenow fragment of DNA polymerase I is active at a temperature of approximately 37° C.

Other non-limiting examples of strand-displacing polymerases are Poly A polymerase, reverse transcriptase, Sequenase, SP6 DNA polymerase, T4 DNA polymerase, T7 DNA polymerase, exo.sup.—Vent (New England Biolabs), exo.sup.—Deep Vent (New England Biolabs), Bst (BioRad), phi29 (New England biolabs), exo.sup.—Pfu (Stratagene), Bca (Panvera), and Sequencing Grade Taq (Promega). The polymerases Tth (Boehringer), Tfl (Epicentre), REPLINASE (DuPont) and REPLITHERM (Epicentre) strand displace from a nick, but also have 5′-3′ exonuclease activity. These polymerases are useful in the presently disclosed methods after removal or decreasing exonuclease activity, e.g., by genetic engineering. The following patent publications describe polymerases that can be used in the methods disclosed herein and are incorporated by reference in their entirety: WO 2007/075987, WO 2007/075873, and WO 2007/076057.

Any one of the isothermal DNA amplifying methods disclosed herein depends on the simultaneous activity of a nicking nuclease and a strand-displacing polymerase. Therefore, the method disclosed herein involves selecting a nicking nuclease, selecting and a strand-displacing polymerase that are compatible (i.e., both enzymes provide substantial activity) at the same conditions (e.g., temperature, pH, divalent cation concentration, and other buffering conditions such as concentration of a particular buffer or salt). Accordingly, in some embodiments, DSN is paired with a Bst polymerase for an amplification reaction run at a temperature of 60-72° C. (e.g., 60 or 65° C.). In some embodiments, DSN, dsDNase, or HL-dsDNase is paired with a Bst polymerase for an amplification reaction run at a temperature of 45-55° C. In some embodiments, DNAse I is paired with Phi29 or Klenow fragment of DNA polymerase I for an amplification reaction run at a temperature of 30-45° C. (e.g., 37° C.). Table 1 provides conditions for activity of various enzymes, as well as conditions under which they may be inactivated.

TABLE 1
Conditions for activation and inactivation of nicking
nucleases and strand-displacing polymerases.

| | | | Optimal | | |
|---|---|---|---|---|---|
| | Optimal | | divalent cation | | Conditions for |
| Enzyme | Temperature | Optimal pH | concentration | | inactivation |
| DSN | 60° C. | 6.6-7.5 | >5 | mM | EDTA, room temperature |
| | | | | | SDS, BME, hydrogen |
| | | | | | peroxide, 60° C. |
| | | | | | >0.2 M NaCl |
| | | | | | Chaotropic agents |
| dsDNase | 20-40° C. | 7.5 | >2.5 | mM | 65° C., 15 min |
| | | | | | 60° C., 20 min |
| | | | | | 1 mM DTT, pH > 8 |
| HL-dsDNase | 20-40° C. | 7.5 | >2.5 | mM | 58° C., 5 min |
| | | | | | 1 mM DTT, pH > 8 |
| DNAse I | 37° C. | 7.5 | >2.5 | mM | vortexing |
| Phi29 | 20-37° C. | 7.5 | >2.5 | mM | EDTA |
| Bst | 60-72° C. | 7.5 | >2.5 | mM | EDTA |
| Klenow fragment | 37° C. | 7.5 | >2.5 | mM | EDTA |
| of DNA | | | | | |
| polymerase I | | | | | |

Another factor influencing the outcome of the isothermal DNA amplification method disclosed herein is the concentration of both enzymes, in absolute values, as well as relative to each other. If there is too much nicking nuclease relative to the strand-displacing polymerase in the reaction mixture, the input DNA could be digested by the nicking nuclease before being copied. If there is too much strand-displacing polymerase relative to the nicking nuclease, adequate amplification may not occur. Table 2 provides some examples of pairs of nicking nucleases and strand-displacing polymerases, their concentrations, and temperatures at which they can be used for isothermal DNA amplification, and the size of the resultant probes that are generated.

The concentration of nicking nuclease can be adjusted so as to alter the average size of the probes generated. For example, if a high concentration (e.g., 1 unit) of nicking nuclease is used, the size of the generated probes is smaller. The concentration of strand-displacing polymerase generally affects the duration of the reaction until saturation is reached. Generally, the higher the concentration of strand-displacing polymerase, the shorter the time needed to reach saturation. Furthermore, incorporation of labeled dNTPs (e.g., biotinylated dUTP) slows the rate of reaction.

TABLE 2
Examples of enzymes and their compatible concentrations and temperatures

| | | | | | | |
|---|---|---|---|---|---|---|
| | | | | | | Range of |
| | | | Concentration | | | average |
| | Concentration | Strand- | of strand- | | | size of |
| Nicking | of Nicking | displacing | displacing | Reaction | Saturation | generated |
| nuclease | nuclease | polymerase | polymerase | Temperature | time | probes |

With biotinylated dUTP

| | | | | | | | | |
|---|---|---|---|---|---|---|---|---|
| DSN | 0.2 | units | Bst | 8 units | 55-70° C. | 4-5 min | 20-50 | bp |
| | | | polymerase | | | | | |
| DSN | 0.1 | units | Bst | 8 units | 55-70° C. | | 30-80 | bp |
| | | | polymerase | | | | | |
| DSN | 0.05 | units | Bst | 8 units | 55-70° C. | | 40-120 | bp |
| | | | polymerase | | | | | |

| | | | | | |
|---|---|---|---|---|---|
| DSN | 0.2 | units | Bst | 4 units | 55-70° C. |
| | | | polymerase | | |
| DSN | 0.1 | units | Bst | 4 units | 55-70° C. |
| | | | polymerase | | |
| DSN | 0.05 | units | Bst | 4 units | 55-70° C. |
| | | | polymerase | | |

Without biotinylated dUTP

| | | | | | |
|---|---|---|---|---|---|
| DSN | 0.2 | units | Bst | 8 units | 55-70° C. |
| | | | polymerase | | |
| DSN | 0.1 | units | Bst | 8 units | 55-70° C. |
| | | | polymerase | | |
| DSN | 0.05 | units | Bst | 8 units | 55-70° C. |
| | | | polymerase | | |
| DSN | 0.2 | units | Bst | 4 units | 55-70° C. |
| | | | polymerase | | |
| DSN | 0.1 | units | Bst | 4 units | 55-70° C. |
| | | | polymerase | | |
| DSN | 0.05 | units | Bst | 4 units | 55-70° C. |
| | | | polymerase | | |

Methods of measuring the specific activity of enzymes is known in the art, and is usually provided by a vendor when an enzyme is obtained from a commercial source. In some embodiments, the activity of a nicking nuclease is measured using the Kunitz assay (M. Kunitz. (1950) J Gen Physiol. 33: 363-377) or a variation thereof, where one unit is defined as the amount of enzyme added to a particular amount (e.g., 50 μg/ml) of calf thymus DNA that causes an increase of 0.001 absorbance units per minute. In some embodiments, a unit of a strand-displacing polymerase is defined as the amount of enzyme that will incorporate a particular amount in nmol (e.g., 0.5 pmol, 10 nmol, or 25 nmol) of dNTP into acid insoluble material in 30 minutes at 65° C. or 37° C.

### Probes

Probes generated by any one of the isothermal DNA amplification methods disclosed herein are a collection, or a library, of dsDNA molecules that have sequences that correspond to, or are shared by the input dsDNA. In some embodiments, probes comprise dsDNA with one blunt end, dsDNA with two blunt ends, and dsDNA with two overhangs. In some embodiments, an overhang is a 3′ overhang. In some embodiments, an overhang is a 5′ overhang.

The average size of the probes is less than the size of the input DNA that is being amplified, and is governed by the concentration of the nicking nuclease relative to the strand-displacing polymerase. The higher the ratio of nicking nuclease to strand-displacing polymerase, the smaller the size (i.e. length) of the probes that are generated. In some embodiments the average size of the probes is expressed as the arithmetic mean of the sizes of all the probes generated by a reaction. In some embodiments, the average size of the probes is expressed as the geometric mean, or the median of the sizes of all the probes generated by a reaction. In some embodiments, the average size of probes is 5-250 bp (e.g., 5-10, 5-15, 5-20, 10-15, 10-20, 10-40, 10-50, 20-50, 5-250, 5-200, 5-150, 5-100, 10-250, 10-200, 10-150, 10-100, 10-80, 10-70, 15-250, 15-200, 15-150, 15-100, 15-80, 15-70, 15-50, 15-40, 20-250, 20-200, 20-100, 40-250, 40-200, 40-150, 40-100, 40-80, 50-100, 50-150, 50-200, 50-250, 70-100, 70-150, 70-200, 70-250, 100-200, or 100-250 bp). In some embodiments, the average size of the probes as generated by any one of the methods disclosed herein is greater than 250 bp (e.g., 250-300, 300-350, 350-400, 250-400, 400-500, 250-500, or 500 bp or more). In some embodiments, more than 50%, 60%, 70%, 80% and even 90% of the probes are between 15-250, 15-120 or 15-70 (inclusive) bp in length. Methods of measuring the average size of a collection of dsDNA is known in the art. For example, a sample of a collection of dsDNA can be run on a size exclusion gel. In some embodiments, size-exclusion chromatography is used to measure the average size of the collection of dsDNA.

In some embodiments, the size of probes generated by any one of the methods disclosed herein is characterized by a median size. In some embodiments, the median size of probes is 5-250 bp (e.g., 5-10, 5-15, 5-20, 10-15, 10-20, 10-40, 10-50, 20-50, 5-250, 5-200, 5-150, 5-100, 10-250, 10-200, 10-150, 10-100, 10-80, 10-70, 15-250, 15-200, 15-150, 15-100, 15-80, 15-70, 15-50, 15-40, 20-250, 20-200, 20-100, 40-250, 40-200, 40-150, 40-100, 40-80, 50-100, 50-150, 50-200, 50-250, 70-100, 70-150, 70-200, 70-250, 100-200, or 100-250 bp). In some embodiments, the median size of the probes as generated by any one of the methods disclosed herein is greater than 250 bp (e.g., 250-300, 300-350, 350-400, 250-400, 400-500, 250-500, or 500 bp or more).

Since dsDNA that is smaller than the input DNA is continuously formed during the course of any one of the isothermal DNA amplification methods disclosed herein, a factor that determines the definition of a generated probe depends on the duration of time that a reaction has run. In some embodiments, “probes” are the dsDNA that are smaller than the input DNA present in the reaction mixture at a time at or after which the reaction has saturated. For example, if an isothermal DNA amplification reaction as disclosed herein saturates in 5 minutes after start of the reaction, all the dsDNA present in the reaction mixture at any time at or after 5 minutes after the start of the reaction that are smaller than the input dsDNA are “probes.” In some embodiments, a reaction is stopped before saturation is reached. In such cases, “probes” are the dsDNA present in the reaction mixture that are smaller than the input dsDNA at a time at or after which the reaction was stopped. Methods of stopping a reaction are discussed below.

As referred to herein, “saturation” means that the reaction is no longer making more nucleic acid, i.e., the mass of the nucleic acid in the reaction has reached steady state. Methods of measuring mass of nucleic acid (e.g., all nucleic acid, or dsDNA) in a sample are known in the art and can also be made in real-time. For example, mass of nucleic acid in a sample can be measured by adding a dye to the sample and measuring a light signal (e.g., fluorescence) of the dye that increases based on the amount of nucleic acid present. Such signals can be read in real-time using instruments such as a plate reader, or a real-time PCR machine. In some embodiment, UV absorbance may indicate the amount of nucleic acid in a sample.

Table 2 provides examples of reaction saturation times for different reaction conditions. In some embodiments, a reaction saturates in 2-20 minutes (e.g., 2-20, 3-20, 4-20, 4-5, 4-6, 4-7, 4-8, 4-9, 4-10, 5-10, 5-15, 5-20, 5-11, 5-12, 8-10, 8-12, 10-12, 10-15, or 15-20 minutes). In some embodiments, a reaction mixture is subjected to a temperature T at which both a nicking nuclease and strand-displacing polymerase is active for a period of 2-20 minutes (e.g., 2-20, 3-20, 4-20, 4-5, 4-6, 4-7, 4-8, 4-9, 4-10, 5-10, 5-15, 5-20, 5-11, 5-12, 8-10, 8-12, 10-12, 10-15, or 15-20 minutes). In some embodiments, a reaction is stopped in 2-20 minutes (e.g., 2-20, 3-20, 4-20, 4-5, 4-6, 4-7, 4-8, 4-9, 4-10, 5-10, 5-15, 5-20, 5-11, 5-12, 8-10, 8-12, 10-12, 10-15, or 15-20 minutes). In some embodiments, a reaction is stopped after it is estimated that saturation is reached. For example, if saturation is expected to be reached 8 minutes after the start of a reaction, the reaction may be run for 10 minutes. Methods of stopping a reaction are discussed below.

Probes formed by any one of the isothermal DNA amplification methods disclosed herein have sequences that are represented by, correspond to, or are shared by the input DNA. In some embodiments, the collection of dsDNA that make up probes formed in a reaction randomly provide a coverage of 50-10,000× (e.g., 50-200×, 50-500×, 50-1000×, 50-1500×, 50-10,000×, 100-200×, 100-1000×, 200-1000×, 500-1500×, 1000-10,000×, 2000-10,000×, 1500-10,000×, or 5000-10,000×) of the input DNA. “Coverage” can be thought of as the number of times a particular base pair of input dsDNA is represented in the formed probes. A coverage of 100× means that, on average, each base pair in the input dsDNA is present in the probes 100 times, or by a count of a 100. A coverage of 100× also means that, on average, each base pair in the input dsDNA has been copied 100× on average. “Coverage” can also be thought of as the “amplification factor,” which is the number of times a base pair or sequence in the input dsDNA has been amplified. Finally, “coverage” may be thought of as represented by the increase in the mass or amount of the dsDNA, resulting from the creation of the probes which are copies of the input dsDNA.

### Input dsDNA and Forming Input dsDNA

In some embodiments, input dsDNA is genomic DNA. In some embodiments, input dsDNA comprise a whole genome of an organism, or is a portion of a whole genome of an organism (e.g., target-enriched genomic DNA). In some embodiments, input dsDNA is cell-free circulating DNA that is fragmented. In some embodiments, input dsDNA is one or more probes (e.g., Ultramers). In some embodiments, input dsDNA from which probes are generated comprises one or more DNA targets (e.g., 2-10,000, 5-10,000, 10-10,000, 20-10,000, 50-10,000, 100-10,000, 500-10,000, 1,000-10,000, 100-200, 100-500, 150-200, 1000-5000, 5000-9000, 6000-8000, 2-1000, 2-100, 10-100, 10-1000, 20-100, 20-500, 10-20, 5-50, 2-5, 2-20, or 5-10 DNA targets), each have a sequence that is different from other DNA targets.

In some embodiments, input dsDNA is on average 40-2000 bp (e.g., 40-100, 40-200, 40-120, 40-2000, 40-1000, 50-200, 100-150, 100-200, 100-500, 100-1000, 100-2000, 500-2000, or 1000-2000 bp) long. In some embodiments, input dsDNA is on average 20-5000 bp (e.g., 20-5000, 20-3000, 20-2000, 40-100, 50-150, 50-200, 100-500, 100-1000, 100-2000, 100-3000, 100-4000, 100-5000, 200-500, 200-1000, 200-2000, 500-1000, 500-2000, 1000-2000, 1000-3000, 2000-4000, 2000-5000, 40-100, 40-200, 40- 120, 40-2000, 40-1000, 50-200, 100-150, 100-200, 100-500, 100-1000, 100-2000, 500-2000, or 1000-2000 bp) long.

While the methods disclosed herein depend on the action of a nicking nuclease on dsDNA, any one of the methods disclosed herein may be applied to ssDNA or RNA by first converting it to dsDNA. Accordingly, any one of the DNA amplification methods disclosed to generate probes from input dsDNA herein comprises, in some embodiments, first forming input dsDNA from input ssDNA or RNA.

ssDNA from which dsDNA is made can be of any length. In some embodiments, input ssDNA is on average 20-5000 bp (e.g., 20-5000, 20-3000, 20-2000, 40-100, 50-150, 50-200, 100-500, 100-1000, 100-2000, 100-3000, 100-4000, 100-5000, 200-500, 200-1000, 200-2000, 500-1000, 500-2000, 1000-2000, 1000-3000, 2000-4000, 2000-5000, 40-100, 40-200, 40- 120, 40-2000, 40-1000, 50-200, 100-150, 100-200, 100-500, 100-1000, 100-2000, 500-2000, or 1000-2000 bp) long.

In some embodiments, a method of forming input dsDNA from input ssDNA comprises use of a DNA nucleotidylexotransferase or terminal transferase (e.g., deoxynucleotidyl transferase).

In some embodiments, a method of forming input dsDNA from input ssDNA comprises forming a reaction mixture comprising:

the input ssDNA,

deoxynucleotidyl transferase (TdT), wherein the TdT has the ability to add a polyA tail to the 3′ end of ssDNA, and

poly-dT-primers, wherein the poly-dT primers consist of equal amounts of a poly-dT-primer with an extra G nucleotide at the 3′ end, a poly-dT-primer with an extra C nucleotide at the 3′ end, and a poly-dT-primer with an extra A nucleotide at the 3′ end; forming a polyA tail on the 3′ end of the ssDNA;

subjecting the reaction mixture to a temperature under which the poly-dT-primers anneal to the polyA tails on the ssDNA; and

subjecting the reaction mixture to a polymerase and to a temperature under which the poly-dT-primers extend to form dsDNA.

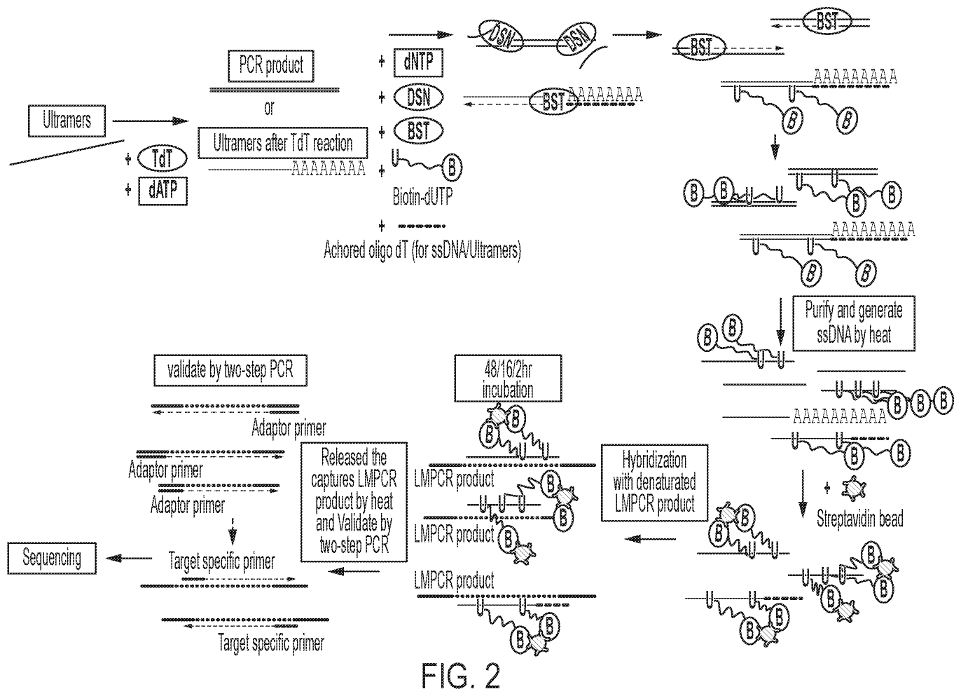

An example of such a method is illustrated in FIG. 2.

In some embodiments, forming input dsDNA from ssDNA comprises performing a Klenow-fragment enzymatic reaction (see e.g., https://www.neb.com/products/m0210-dna-polymerase-i-large-klenow-fragment # Product %20Information) on the input ssDNA. In some embodiments, a Klenow-fragment enzymatic reaction is performed in the presence of random oligonucleotides (e.g., random hexamers or random decamers).

In some embodiments, a method of generating probes as disclosed herein comprises forming input dsDNA from single-stranded nucleic acid by adding one or more oligonucleotides that are complementary to part (e.g., 10-150 nucleotides long) of the single-stranded nucleic acid sequences, whereby oligonucleotide extension and thus formation of dsDNA occurs via polymerase reaction. In some embodiments, a polymerase reaction occurs via a strand-displacing polymerase. In some embodiments, a polymerase reaction to form dsDNA from single-stranded nucleic acid is performed before probe generation. In some embodiments, a polymerase reaction to form dsDNA from single-stranded nucleic acid occurs via a strand-displacing polymerase simultaneously with probe generation.

Accordingly, in some embodiments, a method of generating probes comprises:

(a) forming a reaction mixture comprising:
input single-stranded nucleic acid (e.g., ssDNA),
oligonucleotides that are complementary to at least a part of the input single-stranded nucleic acid,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and

(b) subjecting the reaction mixture to the temperature T under which both the nicking nuclease and the strand-displacing polymerase are active, thereby forming the probes.

In some embodiments, ssDNA is formed by denaturing dsDNA and used as input along with one or more oligonucleotides that are complementary to part (e.g., 2-30 bp) of the single-stranded nucleic acid sequences, so that probes formed by any one of the methods disclosed herein for generating probes have sequences specific to the oligonucleotides added.

Accordingly, any one of the methods for generating probes from a sample of input dsDNA comprises:

(a) forming a reaction mixture comprising:
the input dsDNA,
oligonucleotides that are complementary to at least a part of one or both strands of the input dsDNA,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and

(b) subjecting the reaction mixture to the temperature T under which both the nicking nuclease and the strand-displacing polymerase are active, thereby forming the probes.

In some embodiments, dsDNA is first denatured to form ssDNA before forming a reaction mixture.

In some embodiments, a method of generating probes involves use of single-stranded nucleic acid (e.g., ssDNA). In some embodiments, a method of generating probes involves use of both dsDNA and single-stranded nucleic acid (e.g., ssDNA).

In some embodiments, a method of generating probes comprises:

(a) forming a reaction mixture comprising:
input dsDNA and/or single-stranded nucleic acid (e.g., ssDNA),
oligonucleotides that are complementary to at least a part of one or both strands of the input dsDNA or single-stranded nucleic acid,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and

deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and

(b) subjecting the reaction mixture to the temperature T under which both the nicking nuclease and the strand-displacing polymerase are active, thereby forming the probes.

In some embodiments, dsDNA is first denatured to form ssDNA before forming a reaction mixture with oligonucleotides, nicking nuclease, and strand-displacing nuclease.

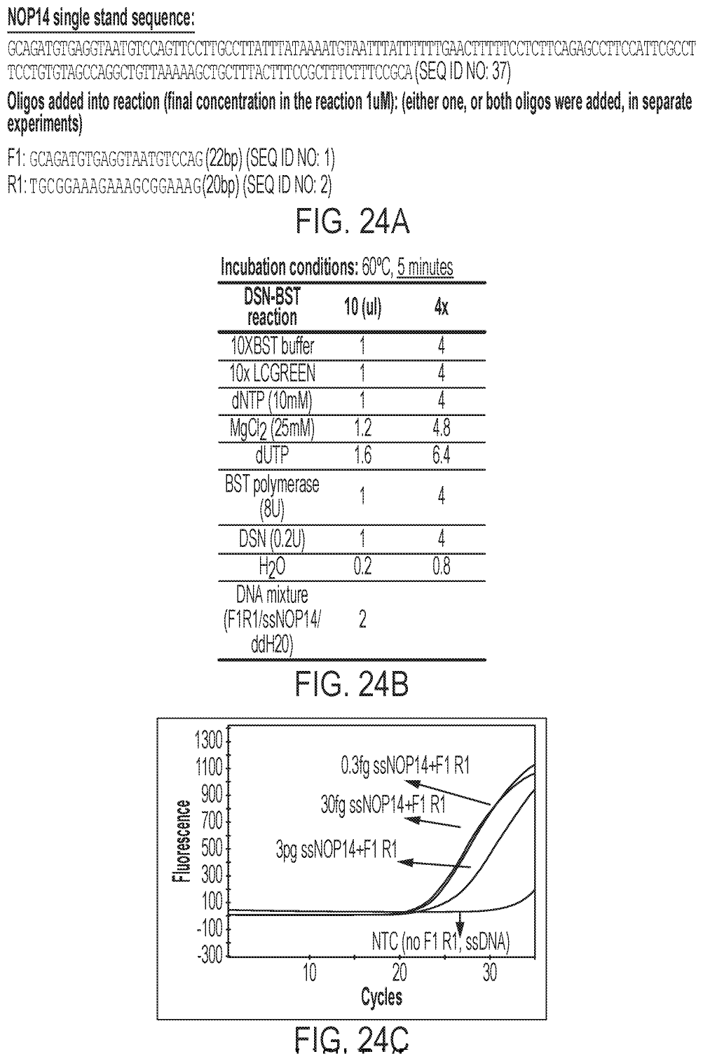

Oligonucleotides to make dsDNA from ssDNA as disclosed herein can be of any sequence that is complementary to a sequence that is to be interrogated, either to make probes to interrogate that sequence in one or more other samples, or to interrogate whether a desired sequence is present in a sample. For example, if it is desired to make probes for a particular gene of interest (e.g., a gene the mutant form of which is implicated in a disease such as cancer), then oligonucleotides that are complementary to the sequence of that gene can be used in conjunction with input ssDNA to make input dsDNA to make dsDNA probes that are complementary to the gene of interest. On the other hand such oligonucleotides can be used to interrogate in a sample of DNA whether the gene of interest is present. In some embodiments, the methods used herein involving oligonucleotides can be used to interrogate whether one or more viruses are present in a biological sample. FIG. 24 provides an example of using oligonucleotides (e.g., F1 and R1 with sequences SEQ ID NOs: 1 and 2, respectively) to make probes using a single strand sequence of NOP14.

In some embodiments, an oligonucleotide as used herein to make dsDNA is 10-150 nucleotides long (e.g., 10-20, 10-30, 10-50, 10-60, 10-100, 20-50, 20-30, 20-100, 40-100, 50-100, 50-120, 50-150, or 100-150 nucleotides long).

In some embodiments, the starting material for the Bst-DSN reaction can be RNA instead of single/double stranded DNA. One of many known methods can be used to form double stranded complementary DNA (cDNA) as input dsDNA from RNA. Accordingly, in some embodiments, input dsDNA for any one of the methods described herein is complementary DNA (cDNA) from RNA.

### Generating Probes in Constrained Spaces

As evident in FIG. 1, the mechanism by which the chain reaction disclosed herein operates depends on the ability of the daughter DNA target strands to re-hybridize once formed. The re-hybridization creates a new double stranded template that propagates the reaction. However, the ability of daughter strands to re-hybridize depends on their concentration. If one starts the reaction with very low amounts of input dsDNA, or if the input dsDNA is highly complex (e.g., mammalian or plant genomic DNA), then the generated daughter strands have very low concentration. This inhibits re-hybridization, and as a result the chain reaction either stops or may amplify debris DNA (e.g., contamination DNA).

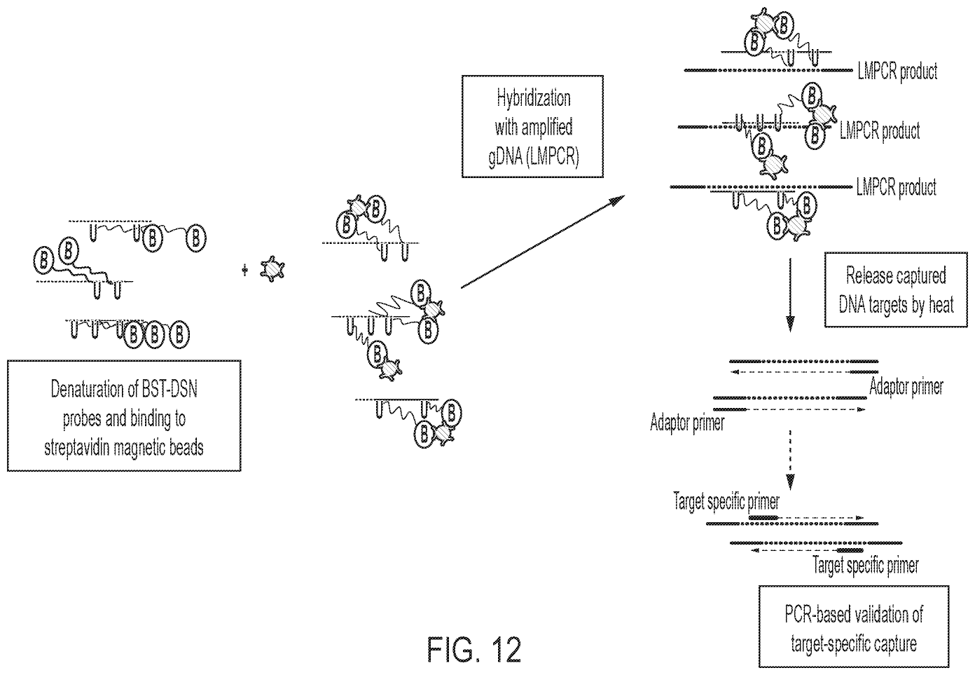

To remedy this problem and to ensure that the daughter strands can find each other even when highly complex DNA is to be amplified, the isothermal DNA amplification chain reaction as disclosed herein can be performed in minute droplets, or emulsion, or nano/pico-litter sized micro-reactors (see e.g., FIG. 12). One example of how to achieve this is water-in-oil droplets as are frequently utilized to perform droplet PCR reactions. When reactions are performed in droplets, then the small size of the droplets ensures that the daughter strands in Bst-DSN reaction will be tightly packed and are likely to hybridize in order to enable propagation of the chain reaction and uniform representation of all targets.

### Enzyme Inactivation and Separation after the Chain Reaction

As discussed above, any one of the methods of isothermally amplifying DNA as disclosed herein can be stopped when, before, or after saturation is reached. There are numerous ways in which a reaction can be stopped. In some embodiments, a reaction is stopped, or slowed substantially (e.g., by 50%, 60%, 70%, 80%, 90%, 95%, 99%, or 99.9% or greater), by inactivating or substantially diminishing the activity of either one or both of the nicking nuclease and strand-displacing polymerase (e.g., reduced by 50% or more, 60% or more, 70% or more, 80% or more, 90% or more, or 99% or more compared to the enzyme's maximum activity). In some embodiments, the activity of either or both the nicking nuclease and strand-displacing polymerase is inhibited or diminished by changing the temperature of the reaction mixture. In some embodiments, the activity of either or both the nicking nuclease and strand-displacing polymerase is inhibited or diminished by subjecting the reaction mixture to a temperature at or above which one or both enzymes are inactivated permanently, e.g., by denaturation (e.g., by exposure to 95° C. or higher). In some embodiments, the activity of either or both the nicking nuclease and strand-displacing polymerase is inhibited or diminished by adding a chelating agent. Non-limiting examples of chelating agents include ethylenediaminetetraacetic acid (EDTA), porphine, vitamin B-12, and dimercaprol.

In some embodiments, the enzymes are separated from the reaction mixture in order to stop their action on the nucleic acid present in the reaction mixture. In some embodiments, enzymes are separated from the reaction mixture by size filtration. Chromatography may also be used to separate the enzymes from the reaction mixture. In cases where the enzymes contain a tag (e.g., a His tag, myc tag, or Flag tag), the enzymes can be immunoprecipitated out from the reaction mixture using an antibody or molecule that binds specifically to the tag.

Similarly, in some embodiments, tagged probes generated in the reaction mixture are separated from the reaction mixture to prevent further enzyme action on them by using an antibody or molecule that binds specifically to the tag. For example, if biotinylated probes are generated, they may be separated from the reaction mixture using streptavidin beads.

### Incorporation of Modified Nucleotides

For formation of a second strand from a nick by a strand-displacing polymerase, deoxynucleotide triphosphates (dNTPs) are required in the reaction. In some embodiments, a reaction mixture comprises one or more the following dNTPs: deoxyadenosine triphosphate (dATP), deoxythymidine triphosphate (dTTP); deoxycytosine triphosphate (dCTP), and deoxyguanosine triphosphate (dGTP). In some embodiments, a reaction mixture comprises dATP, dTTP, dCTP, and dGTP.

Incorporation of a labeled nucleotide (e.g., biotinylated uracil) by using biotin-dUTP in the place of dTTP, or in addition to the four natural nucleotides in the amplification reaction results in formation of labeled (e.g., biotinylated) probes that can be bound directly to streptavidin solid support, such as streptavidin magnetic beads that are often used for hybrid capture reactions. FIG. 2 demonstrates the full workflow that can be used to generate biotinylated amplified probes via any one of the methods disclosed herein, bind those to beads, and then use the beads to attract specific targets from human genomic DNA (or from whole-genome-amplified genomic DNA, or ligation-mediated PCR, LMPCR-amplified whole genomic DNA).

Accordingly, in some embodiments, a reaction mixture of any one of the methods disclosed herein comprises dATP, dTTP, dCTP, dGTP, and biotin-dUTP. In some embodiments, dTTP is replaced by biotin-dUTP and the reaction mixture comprises dATP, biotin-dUTP, dCTP, and dGTP.

Additional modified nucleotides may also be incorporated in the amplified probes during the reaction. For example, 2,6 di-amino-purine may be incorporated into probes via a strand-displacing polymerase (e.g., a Bst polymerase), replacing adenosine in the generated DNA probes and will provide a higher melting temperature Tm for the generated probes.

Accordingly, in some embodiments, a reaction mixture of any one of the methods disclosed herein comprises dATP, dTTP, dCTP, dGTP and 2,6 di-amino-purine. In some embodiments, dATP is replaced by 2,6 di-amino-purinetriphosphate and the reaction mixture comprises 2,6 di-amino-purinetriphosphate, dTTP, dCTP, dGTP.

In some embodiments, inosine replacing guanine lowers the Tm. Accordingly, in some embodiments, a reaction mixture of any one of the methods disclosed herein comprises inosinetriphosphate. In some embodiments, dGTP is replaced by inosinetriphosphate and the reaction mixture comprises dATP, dTTP, dCTP, and inosinetriphosphate. In some embodiments, the reaction mixture comprises dATP, dTTP, dCTP, dGTP and inosinetriphosphate.

In some embodiments, a reaction mixture comprises both biotin-dUTP, as well as inosinetriphosphate or 2,6 di-amino-purinetriphosphate to both incorporate a tag in the generated probes and also increase/decrease their melting temperature.

### Validating Formed Probes

The probes generated by any one of the isothermal DNA amplification methods disclosed herein can be validated for use in a number of ways. Since the probes are a library of dsDNA molecules that have sequences that correspond to, or are shared by the input dsDNA, one way to validate them is to simply sequence them. Another way in which to validate them is to test whether they are able to bait, or bind to target nucleic acids.

In some embodiments, validated probes formed in any one of the methods disclosed herein comprises:

attaching the probes to a solid surface;

incubating the probes with a sample of target DNA fragments to allow hybridization of the probes and target DNA sequences, wherein each target DNA fragment is ligated to an adapter;

washing away unbound DNA fragments;

releasing the target DNA fragments that are hybridized to the probes;

amplifying the released target DNA fragments using primers that are complementary to the adapters;

amplifying the released target DNA fragments using target-specific primers; and

interrogating (e.g., by sequencing) the amplified released target DNA fragments to determine whether the amplified released target DNA fragments are specific to the probes.

Such a validation method is illustrated in FIG. 2.

### Use of Probes

Provided herein is a method of interrogating target DNA regions in a sample. In some embodiments, probes generated by any one of the isothermal amplification methods as disclosed herein are used to as bait to capture target DNA sequences in a sample of DNA suspected to have one or more of the target DNA regions. In some embodiments, a method of interrogating target DNA regions in a sample of DNA comprises:

(a) providing one or more input probes (e.g., Ultramers), wherein each input probe is a dsDNA, each single strand of which is complementary to a target DNA region, wherein the target DNA region for each input probe is different from the target DNA region for all other input probes;

(b) generating probes according of any one of isothermal DNA amplification methods disclosed herein, wherein each of the input probes serves as an input dsDNA;

(c) incubating a first aliquot of the generated probes with a sample of DNA suspected to have one or more of the target DNA regions to allow the generated probes to capture complementary target DNA regions;

(d) amplifying the captured target DNA regions; and

(e) performing sequencing on the captured target DNA sequences.

In some embodiments, captured target DNA regions are released before being sequenced (e.g., by raising the temperature above the melting temperatures of all the probe-target duplexes).

In some embodiments, the efficiency of capture is characterized by the percentage of specific or desired target sequences captured versus the percentage of off-target or non-specific sequences that are captured. An “on-target percentage” is the percentage of captured DNA that is specific or desired. For example, if the on-target percentage for a given capture reaction is 20%, then this means that 20% of all the sequences captured in the reaction were specific or desired, while 80% of the captured sequences are non-specific or off-target. “On-target percentage” can also be thought of as the percentage of captured DNA targets that are true positives and not false positives.

In some embodiments, the on-target percentage after a round of capture using probes generated using any one of the methods disclosed herein is less than 20% (e.g., less than 20%, less than 15%, less than 10%, less than 5%, less than 4%, less than 3%, less than 2%, less than 1%, less than 0.8%, less than 0.6%, less than 0.4%, less than 0.2%, or less than 0.1%).

In some embodiments, the on-target percentage of a capture reaction performed using any of the generated probes described herein after one round of capture is less than the on-target percentage of a capture reaction performed under substantially the same conditions but using the input dsDNA from which the probes are made (e.g., commercially available probes that are longer than the generated probes) as capture probes. To improve the rate of on-target percentage, at least another round of capture (e.g., a total of 2 or more, a total of 3 or more, a total of 4 or more, or a total of 5 or more rounds of capture) can be performed using a another (e.g., a second, a third, a fourth, or a fifth) aliquot of generated probes. Accordingly, in some embodiments, a method of interrogating target DNA regions in a sample of DNA comprises, after a first round a capture using a first aliquot of generated probes, releasing the captured complementary target DNA regions from the generated probes from the probes in the first aliquot of generated probes, amplifying the released target DNA regions, and incubating the amplified capture target DNA regions with a second aliquot of the generated probes. In some embodiments, a second round of capture is carried out without amplifying the released nucleic acid from the first round of capture.

In some embodiments, the first and second aliquots of the generated probes are the same. In some embodiments, the first and second aliquots of the generated probes are different, but from the same composition of generated probes, so that the composition of the first and second aliquots is similar (e.g., the average size and concentration of probes is the same). In some embodiments, a second round of capture is performed using an aliquot of generated probes that is sourced from a different source than that of the first aliquot, such that the composition of the first and second aliquots is different. For example, the first and second aliquots of generated probes can have different average sizes of probes, different sequences because the input dsDNA had different sequences, or different concentration of probes. In some embodiments the size of a first and second aliquot of generated probes can be different (e.g., a first aliquot can be 10 μl while a second aliquot may be 20 μl). In some embodiments, a second round of capture is followed by releasing captured complementary target DNA regions from probes of the second aliquot of generated probes, and amplifying the released target DNA regions of the second aliquot of generated probes before analysis (e.g., by sequencing).

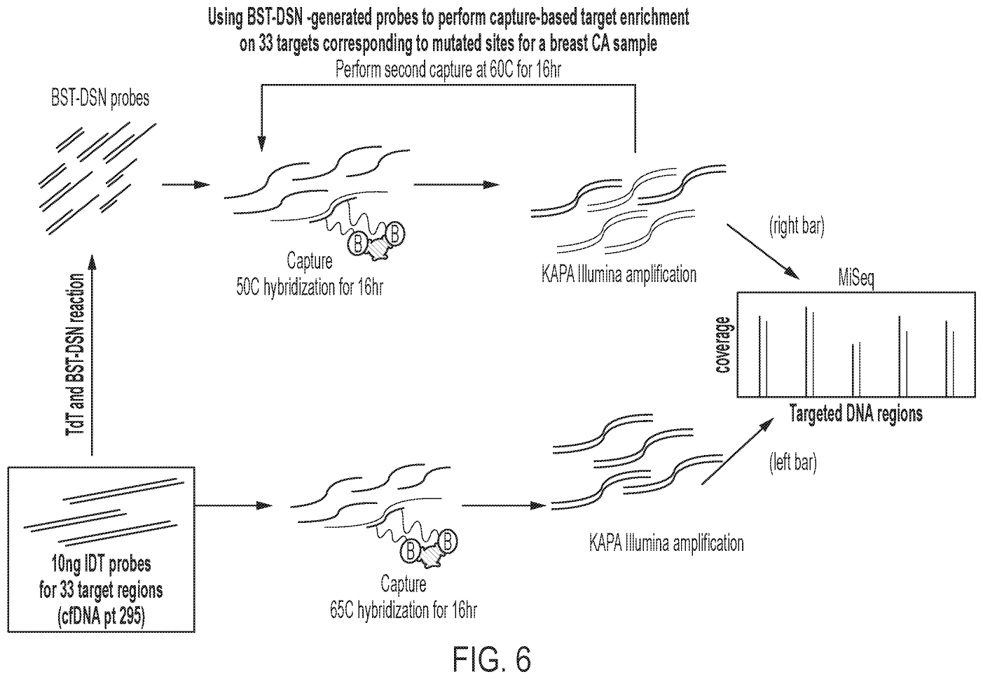

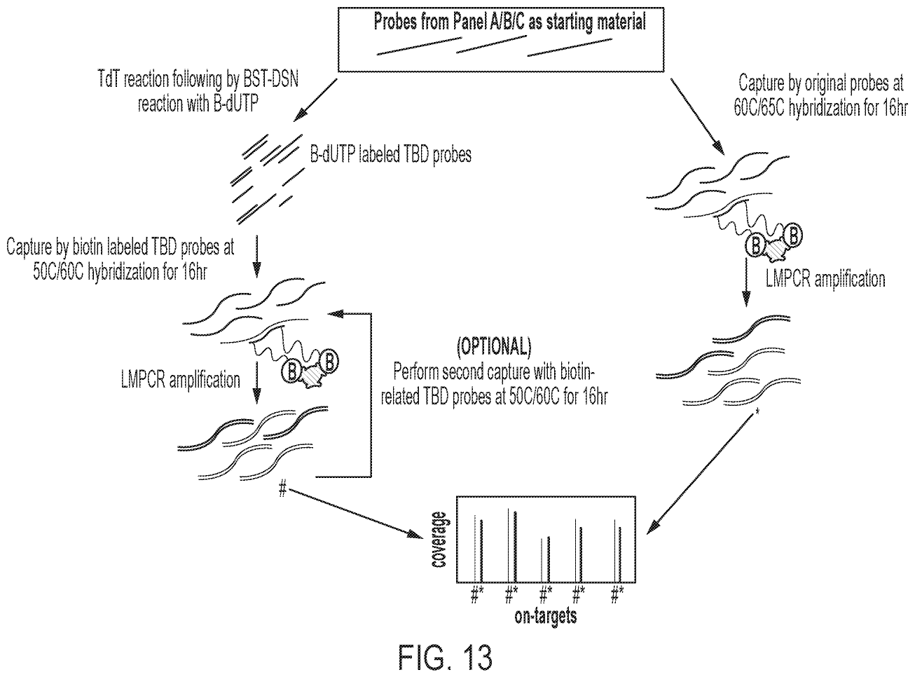

Examples of interrogating target DNA regions in a sample of DNA using probes generated by isothermal amplification as disclosed herein are provided in FIG. 6 and in FIG. 13.

In some embodiments, the on-target percentage after a second round of capture is 15-99% (15-20, 20-30, 30-40, 40-50, 50-60, 60-70, 70-80, 80-90, 15-30, 15-50, 20-40, 20-50, 20-60, 30-50, 30-60, 30-80, 50-70, 50-80, 50-90, 60-80, 60-90, 60-95, 90-95, 95-99, 50-95, 50-99, or 90-99%). In some embodiments, the on-target percentage after two rounds of capture with probes generated using isothermal amplification methods as disclosed herein is greater than one round of capture with input dsDNA that was used to make the generated probes.

A method of interrogating target DNA regions may include a second capture. Accordingly, a method of interrogating target DNA regions may further comprise releasing captured complementary target DNA and incubating the released complementary target DNA with a fresh aliquot of the probes to allow the probes in the fresh aliquot to capture complementary target DNA regions.

In some embodiments input probes are made from ssDNA, or RNA as described above. In some embodiments, a sample of DNA comprises genomic DNA, which may be obtained from a biological sample. In some embodiments, genomic DNA is that of a micro-organism and the method is used to identify the species of the micro-organism. In some embodiments, genomic DNA is from a subject suspected of having one or more mutations in one or more target regions.

### Composition of Probes

The present disclosure provides a composition comprising probes that are a library of dsDNA molecules that have sequences that represent, correspond to, or are shared by an input dsDNA, or a template DNA, RNA or other nucleic acid. The probes of the composition have characteristics that are described above. In some embodiments, a composition of probes as described herein comprises one or more buffering components such as buffering agents (e.g., Tris-HCl, borate, acetic acid, or citric acid) and salts (e.g., NaCl, or KCl). In some embodiments, a composition of probes comprises stabilizing agents and preserving agents (e.g., tehalose).

In some embodiments, a composition of probes is freeze-dried. In some embodiments, a composition of probes is deposited on an FTA Card.

### Kit

Provided herein is a kit comprising one or more nicking nucleases and one or more strand-displacing polymerases. In some embodiments, such a kit further comprises deoxynucleotide triphosphates (dNTPs), which may comprises one or more of the following dNTPs: deoxyadenosine triphosphate (dATP), deoxythymidine triphosphate (dTTP); deoxycytosine triphosphate (dCTP), and deoxyguanosine triphosphate (dGTP). In some embodiments, a kit comprises dNTPs that are modified, for example, labeled dNTPs (e.g., biotin-dUTP), and dNTPs that can alter the melting temperature of probes into which they are incorporated (e.g., inosinetriphosphate, and 2,6 di-amino-purinetriphosphate). In some embodiments, a kit comprises divalent salts (e.g., MgCl₂) that can be used to adjust the activity of a nicking nuclease and/or a strand-displacing nuclease. In some embodiments, a kit as provided herein includes reagents to inactivate or diminish the activity of enzymes (e.g., DTT, or bME). In some embodiments, a kit comprises pre-made buffering stock solutions that can be added to input dsDNA, and one or more strand-displacing polymerases to start a reaction. A kit may also provide instructions, or guidance, of how to pair a nicking nuclease and a strand-displacing polymerase with recommended reaction temperatures, pH, and reaction compositions.

### Reaction Mixture

Provided herein is a reaction mixture comprising a nicking nuclease, a strand-displacing polymerase, and dNTPs as described above and elsewhere in this disclosure. In some embodiments, a reaction mixture comprises DNA and a Bst polymerase. In some embodiments, a reaction mixture comprises Shrimp-based double strand specific nucleases (dsDNase) or HL-dsDNAse, and a Bst polymerase. In some embodiments, a reaction mixture comprises DNAse I and a Klenow fragment of DNA polymerase I. In some embodiments, a reaction mixture comprises DNAse I, phi29 polymerase, and a Klenow fragment of DNA polymerase I. dNTPs may comprise one or more of the following dATP, dGTP, dCTP, dTTP, or analogs thereof. In some embodiments, dNTPs comprise iosinetriphosphate, or 2,6 di-amino-purinetriphosphate.

In some embodiments, a reaction mixture comprises any one of the reaction mixtures described in Table 2. In some embodiments, a reaction mixture may comprise any one of the reaction mixtures described in Table 2 at a temperature listed in Table 2.

A reaction mixture may comprise one or more buffering components such as buffering agents (e.g., Tris-HCl, borate, acetic acid, or citric acid), salts (e.g., NaCl, or KCl), and/or divalent cations (e.g., Mg²⁺), or other components that are compatible with both a nicking nuclease and strand-displacing polymerase that are present in the reaction mixture.

### Whole Genome Amplification for Identification of Microorganisms and Identification of Mutants

In addition to being used to make probes for application such as hybrid capture, the disclosed isothermal DNA amplification method may be used to amplify whole genomes from any species including micro-organisms, such as bacteria, and viruses, which can then be used for sequencing analysis. Following DNA amplification by any one of the methods disclosed herein on a sample of dsDNA that may potentially contain micro-organisms, the generated amplified short-DNA (probes) can be used for sequencing to identify the presence of any unknown micro-organism. To do this, an end-repair reaction followed by ligation of sequencing primers can be used by employing standard commercial kits, such as the NEB Next II system for Illumina sequencing (New England Biolabs).

Another application of the disclosed isothermal DNA amplification methods pertains to generating amplified DNA of short size using a clinical DNA sample. For example, using patient-derived genomic DNA (e.g., obtained from biopsies, FFPE DNA, blood, urine, cheek swab, a bronchial lavage, or pulmonary brushing, or circulating DNA) as input dsDNA, short versions of the same DNA sample can be produced using any one of the methods disclosed herein. Such short size ‘target’ DNA may be useful in combination with mutant enrichment and identification methods such as ‘Nuclease Assisted Mutation Enrichment with probe overlap (NAME-PRO). Therefore, probes generated using any one of the method disclosed herein are capable of capturing both wild-type and mutant target nucleic acid molecules.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Generation of dsDNA Probes Using a Bst-DSN Chain Reaction, and Use of the Generated Probes to Enrich Target Sequences

FIG. 1 illustrates the basic principle of using the Bst-DSN chain reaction for DNA amplification. The combination of Bst and DSN is ideal, as both enzymes work optimally at similar temperatures (e.g., 65° C.) and buffer conditions, while the reaction components required for Bst (dNTPs, NaCl and Mg++ content) are compatible with DSN activity.

### Generation of Labeled Probes

One application of this method is to generate probes that may be used for DNA hybrid capture. To this end, probes that have a tag via which the probes may be immobilized can be very useful. For example, incorporation of a biotinylated nucleotide such as biotin-dUTP in the place of dTTP, or in addition to the four natural nucleotides in the Bst-DSN reaction results in generation of biotinylated probes that can be bound directly to streptavidin solid support, such as streptavidin magnetic beads that are often used for hybrid capture reactions. FIG. 2 demonstrates a workflow that can be used to generate biotinylated amplified probes via Bst-DSN reaction, bind those to streptavidin beads, and then use the beads to attract specific targets from human genomic DNA (or from whole-genome-amplified genomic DNA, or ligation-mediated PCR (LMPCR)-amplified whole genomic DNA).

### Conversion of Single-Stranded DNA to Double-Stranded DNA

While the method described in FIG. 1 requires dsDNA as a template, the Bst-DSN chain reaction can be applied to ssDNA or RNA by first converting them to dsDNA. FIG. 2 demonstrates the specific case where the starting DNA comprises single stranded DNA from which amplified probes are to be generated. Before the Bst-DSN reaction is initiated, the single stranded DNA is converted to double stranded DNA via a terminal deoxynucleotidyl transferase (TdT) reaction, which adds a polyA tails at the 3′ end of the single stranded molecule. Following this, a poly-dT primer with an extra X nucleotide at the 3′ end (X=G, C, and A in equal amounts) is added to the reaction, and the Bst-DSN reaction can be initiated. In the first instance, Bst extends the poly-dT primer to generate double stranded DNA following which the Bst-DSN reaction can proceed as usual.

There are many known alternatives to the TdT method for converting ssDNA to dsDNA. For example, dsDNA may be generated using a Klenow-fragment enzymatic reaction with random hexamers that generate a second DNA strand from the single-stranded DNA ‘Ultramer’.

### Using Bst-DSN Reaction Generated Probes for Hybrid-Capture

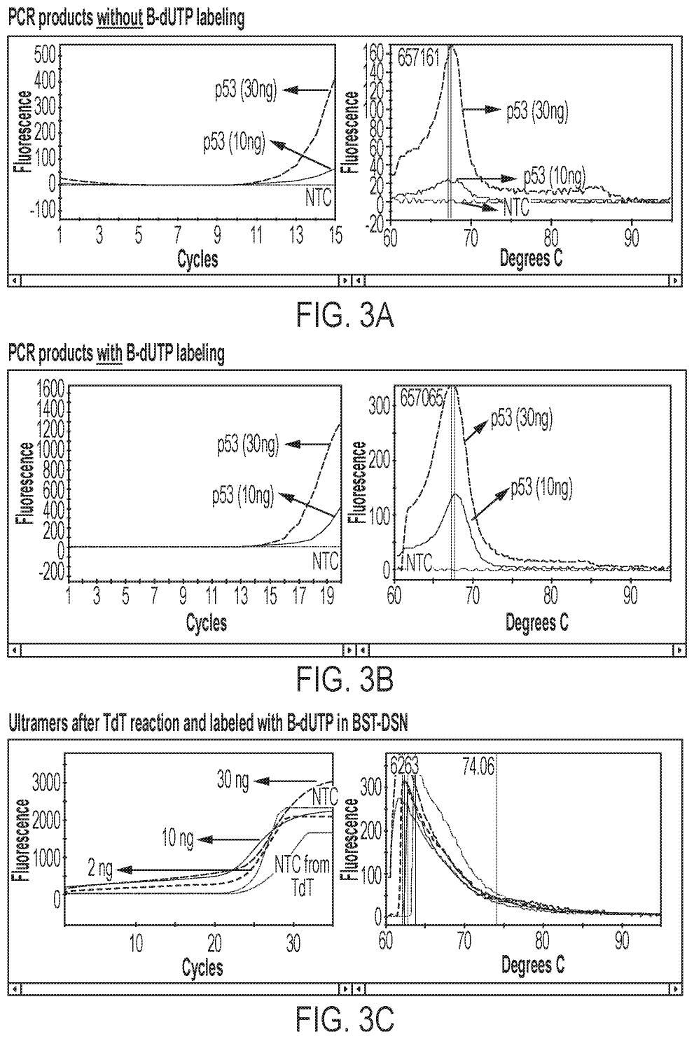

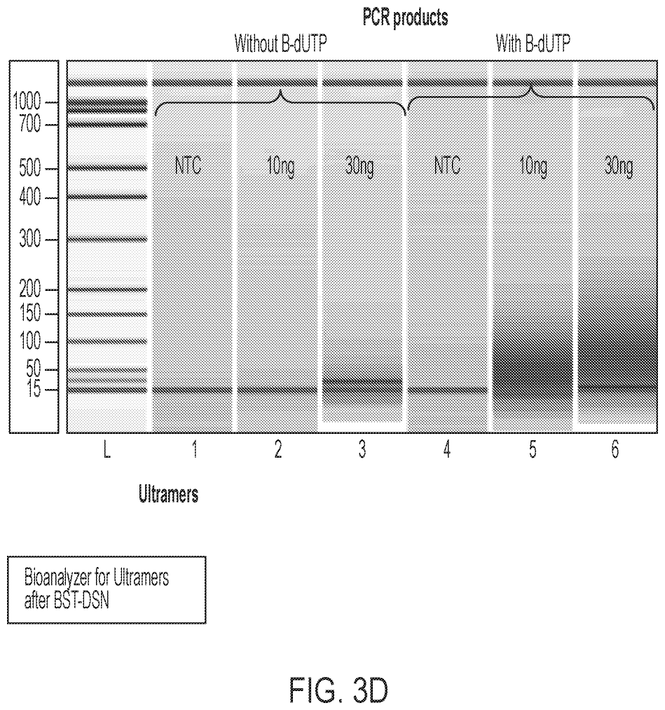

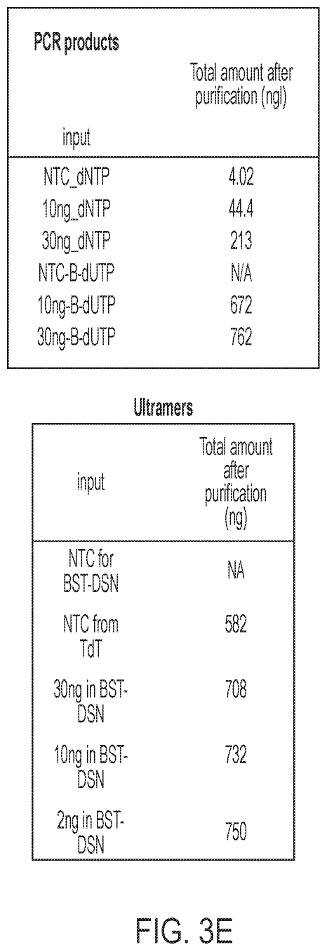

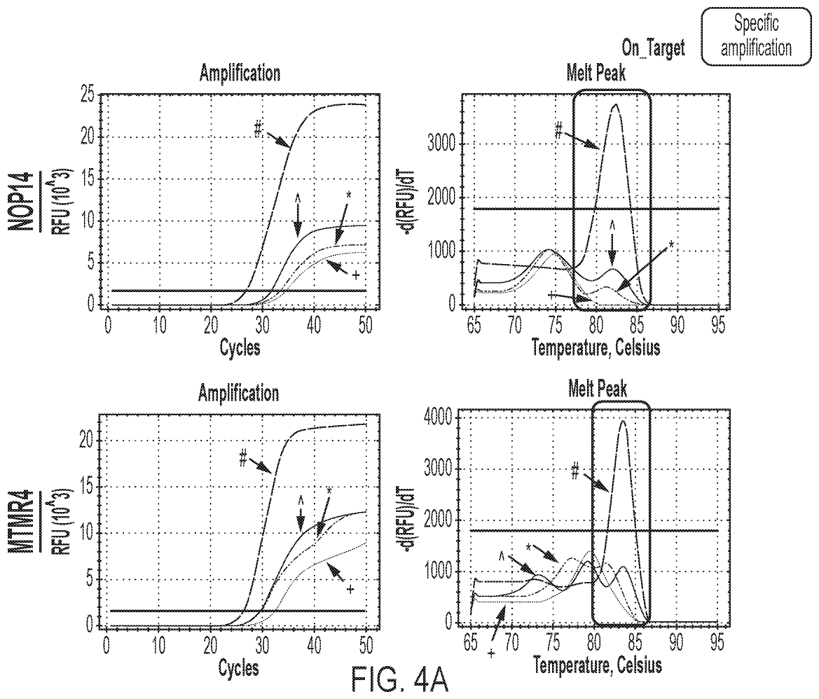

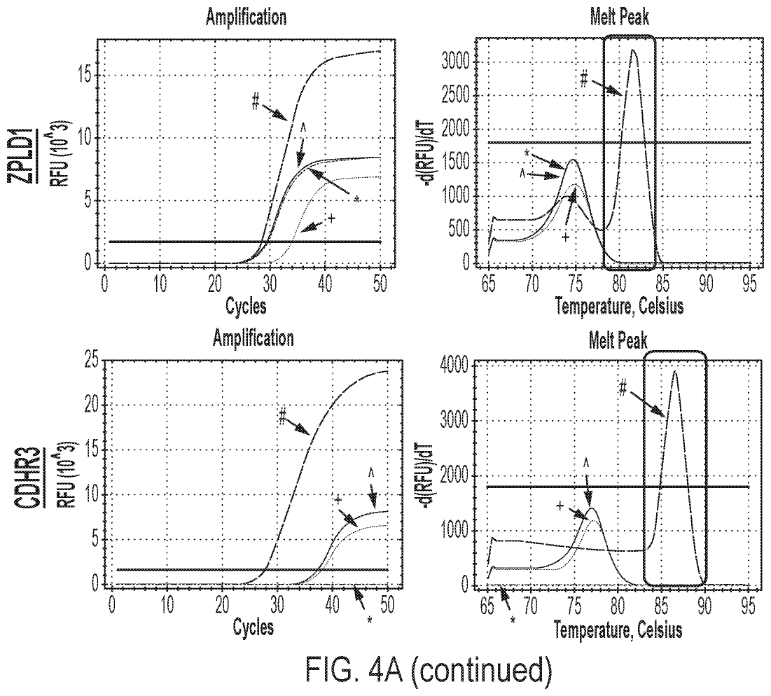

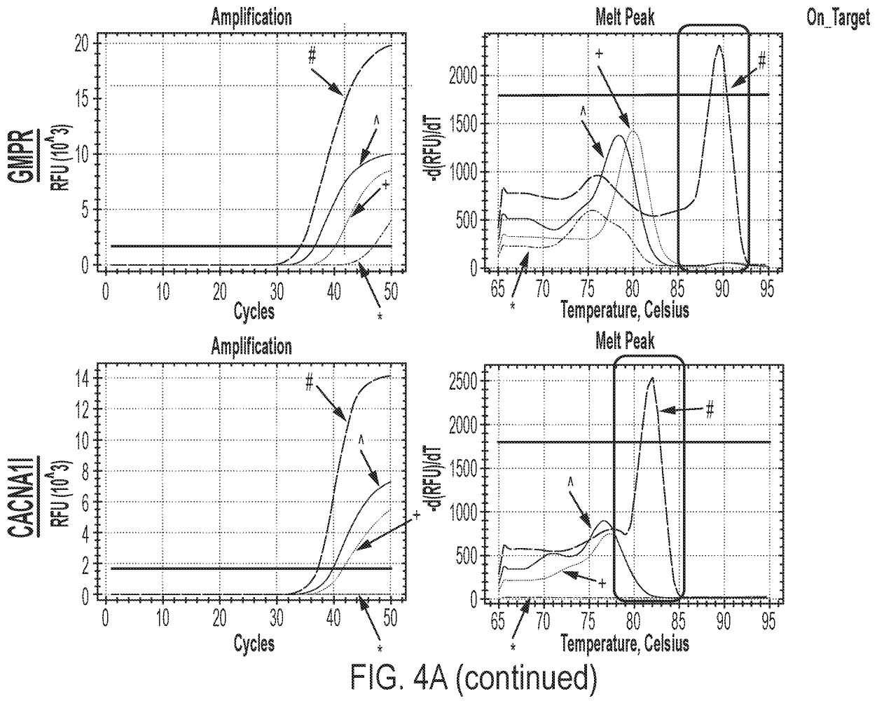

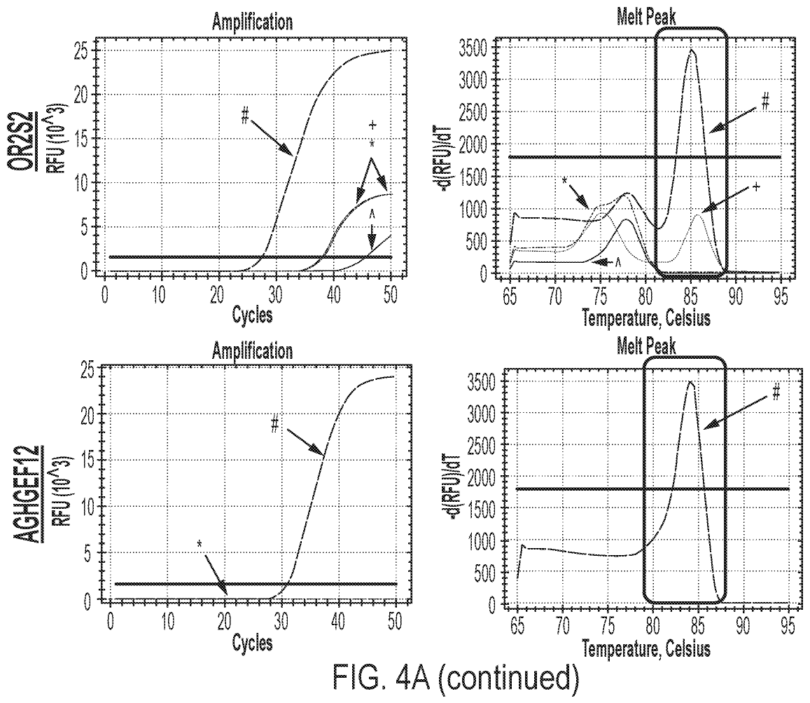

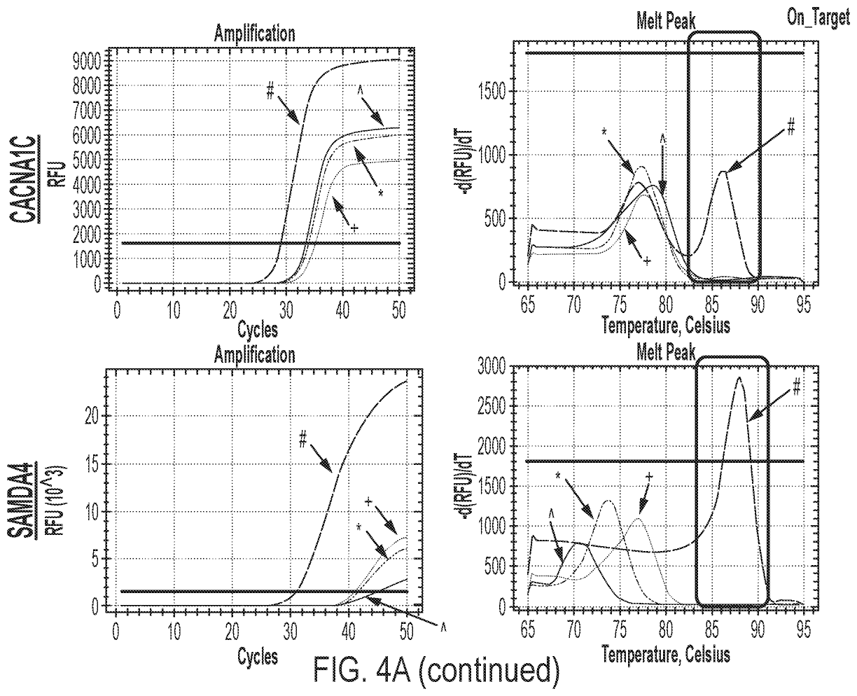

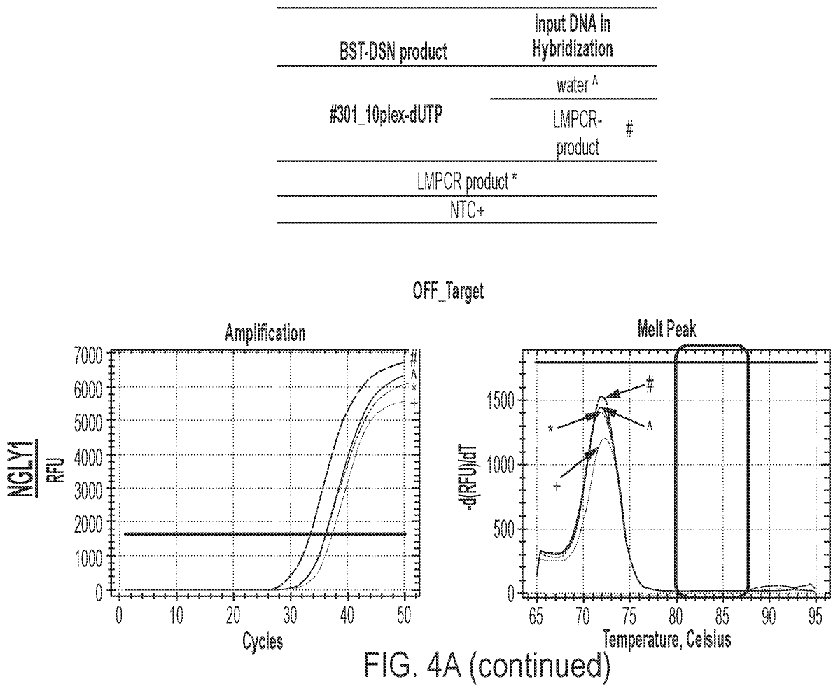

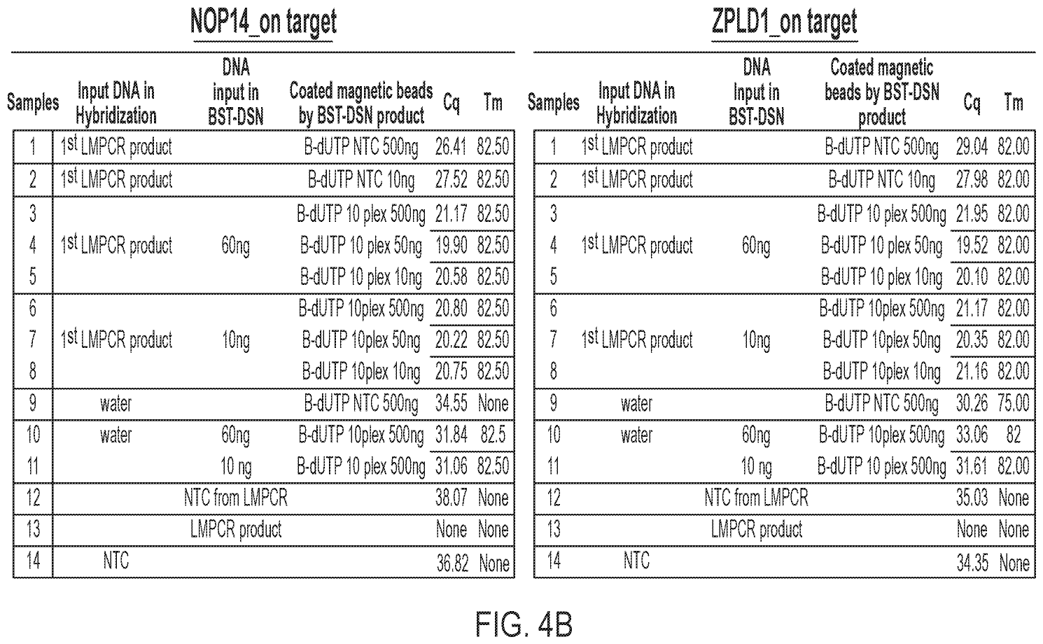

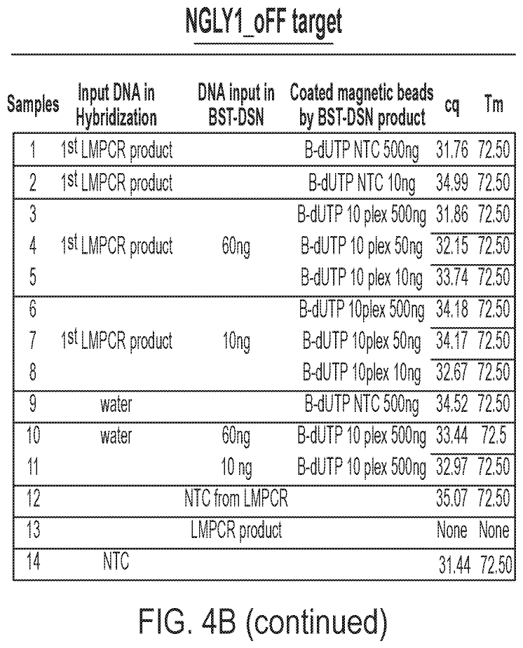

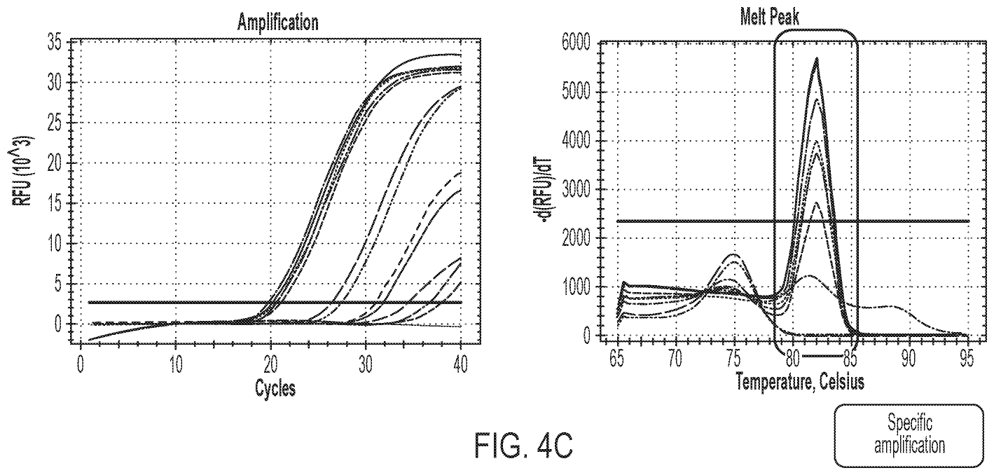

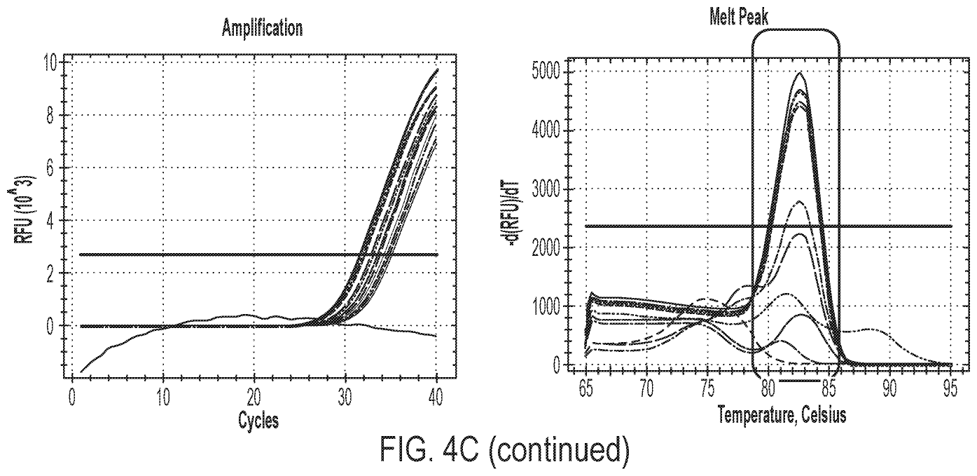

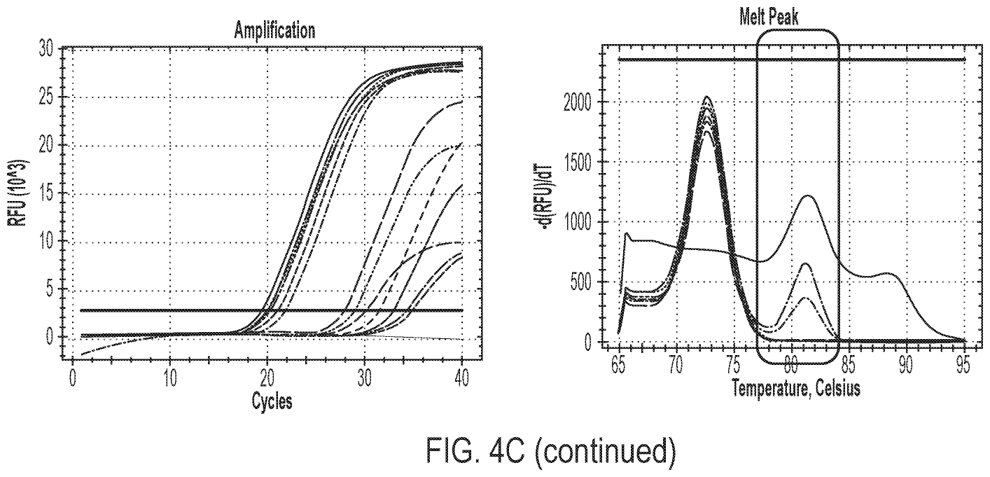

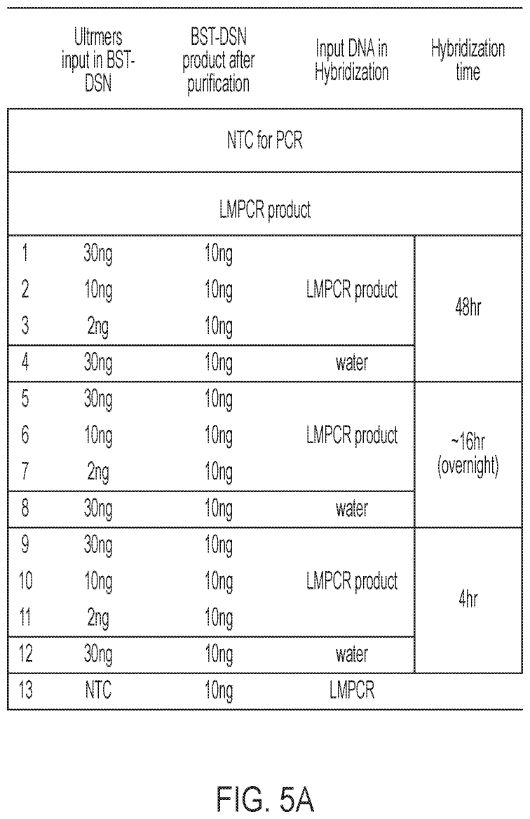

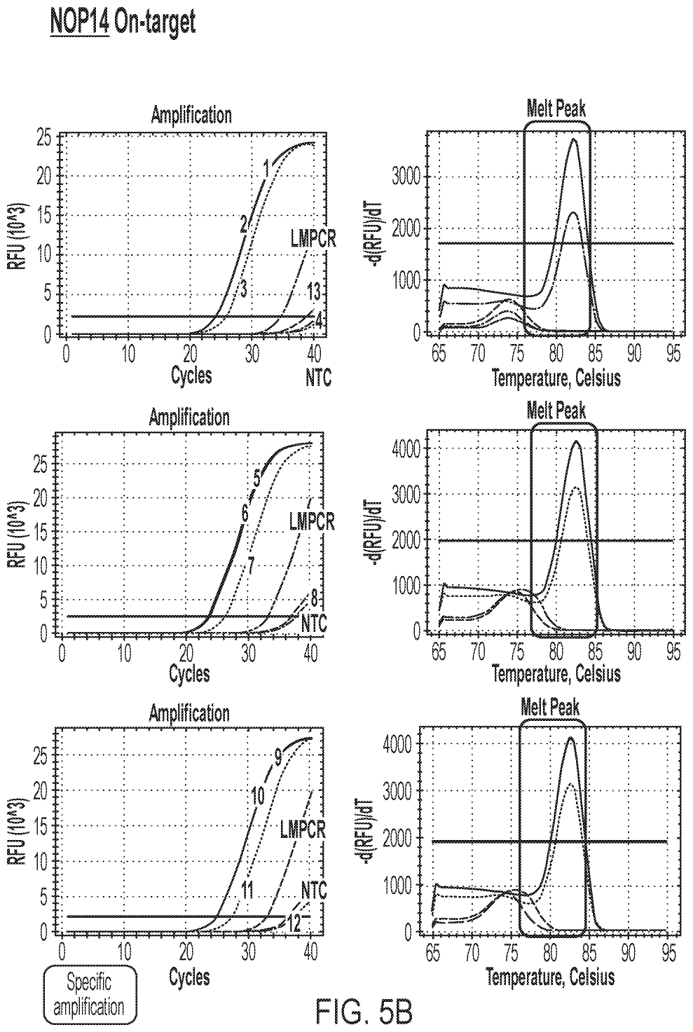

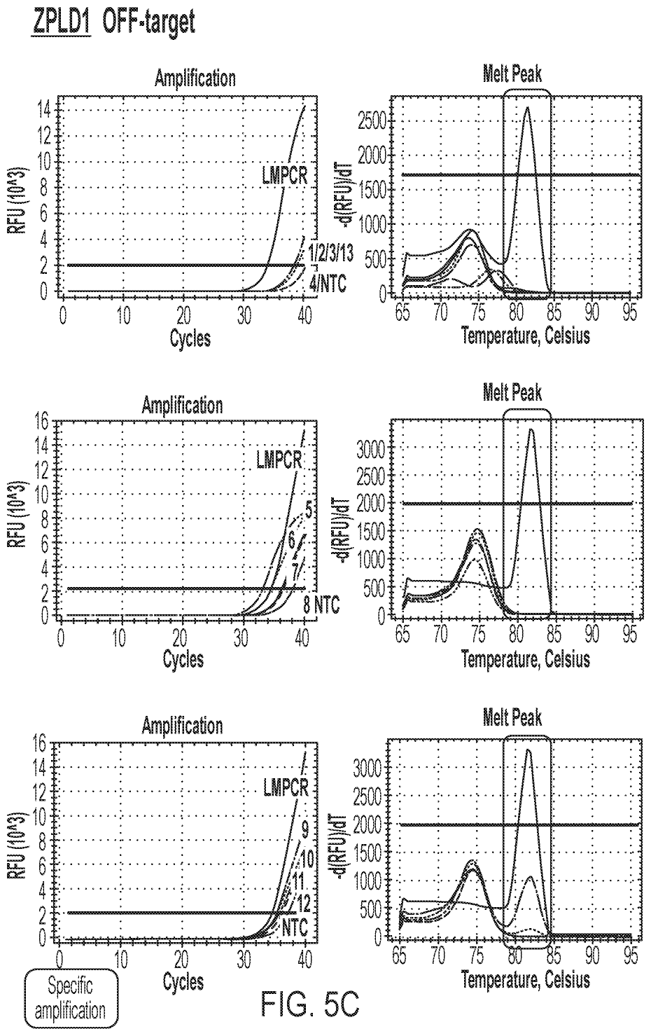

FIGS. 3-6 show results where the Bst-DSN reaction is used to generate biotinylated amplified probes using as starting material (a) 10 individually amplified PCR products which have been mixed in a single sample; (b) a single stranded Ultramer (120 bp long) from the NOP gene which was first converted to dsDNA via a TdT tailing reaction, and then used to generate NOP gene biotinylated capture probes that capture the corresponding sequences from amplified human genomic DNA—LMPCR product.

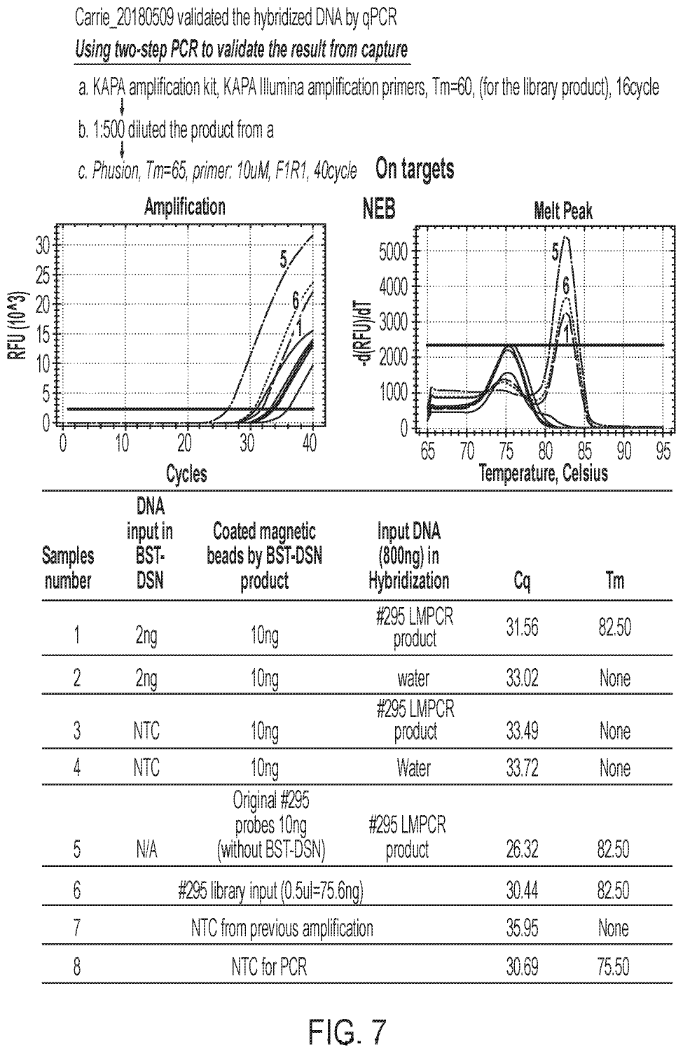

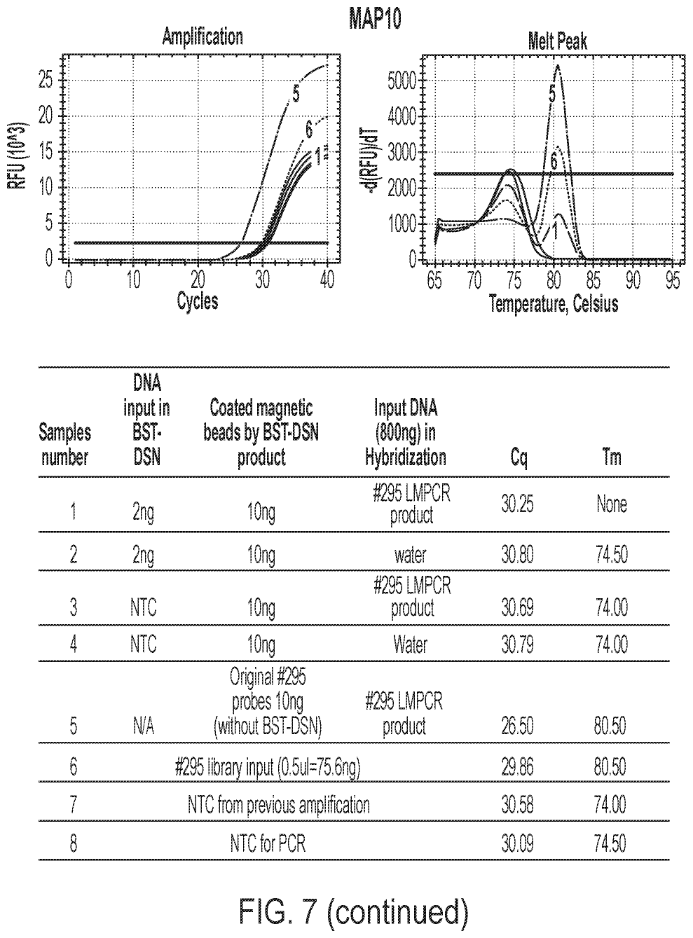

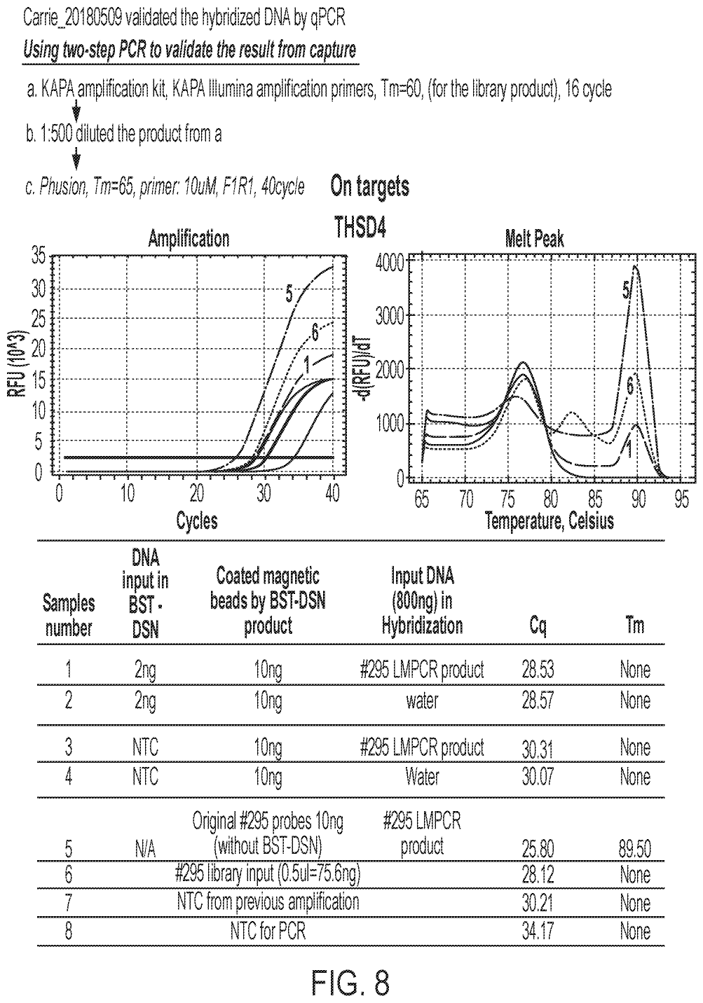

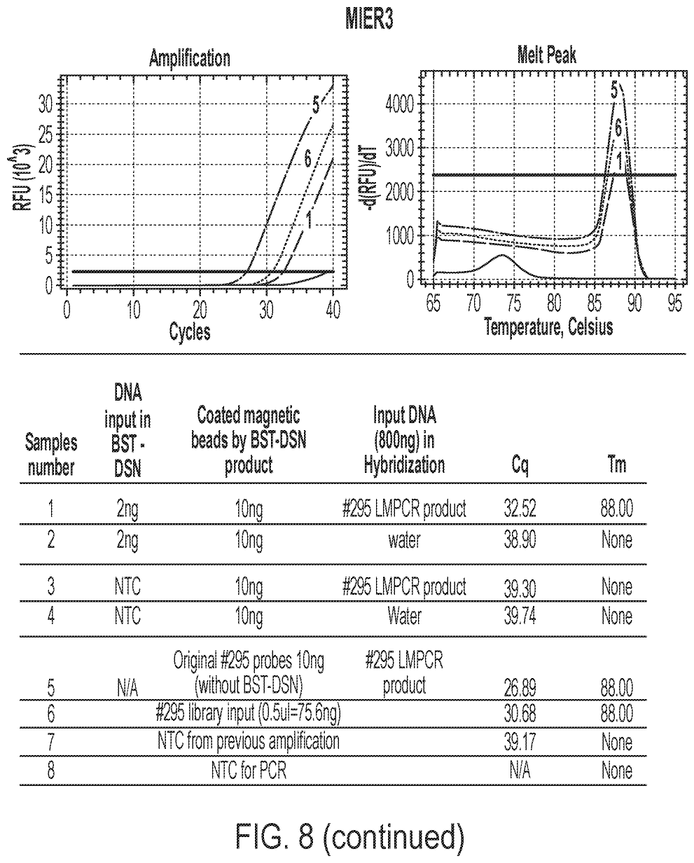

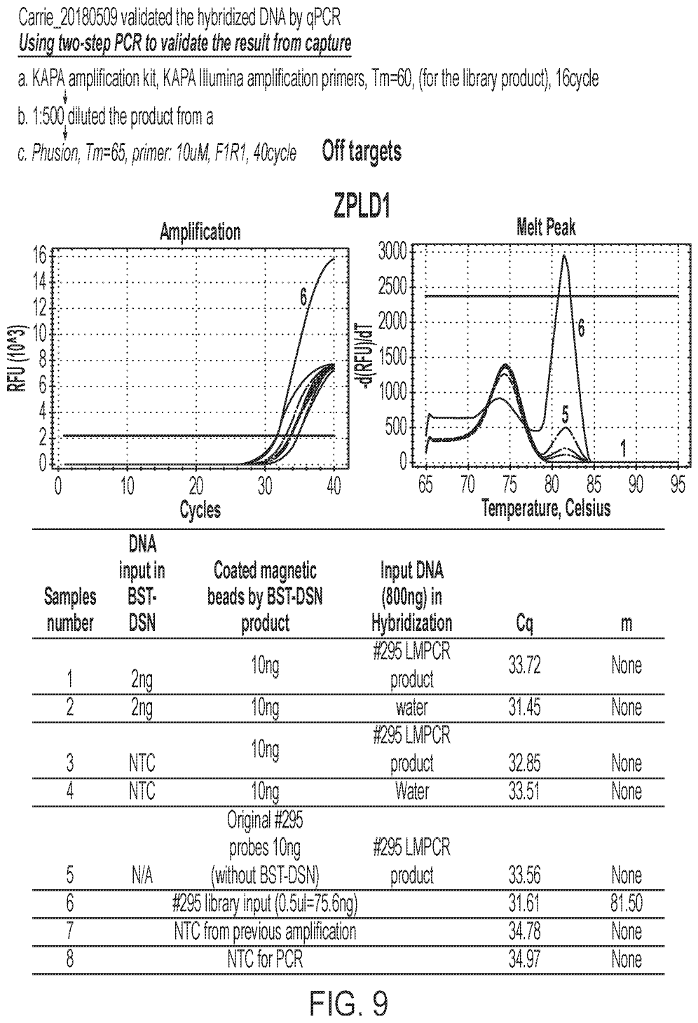

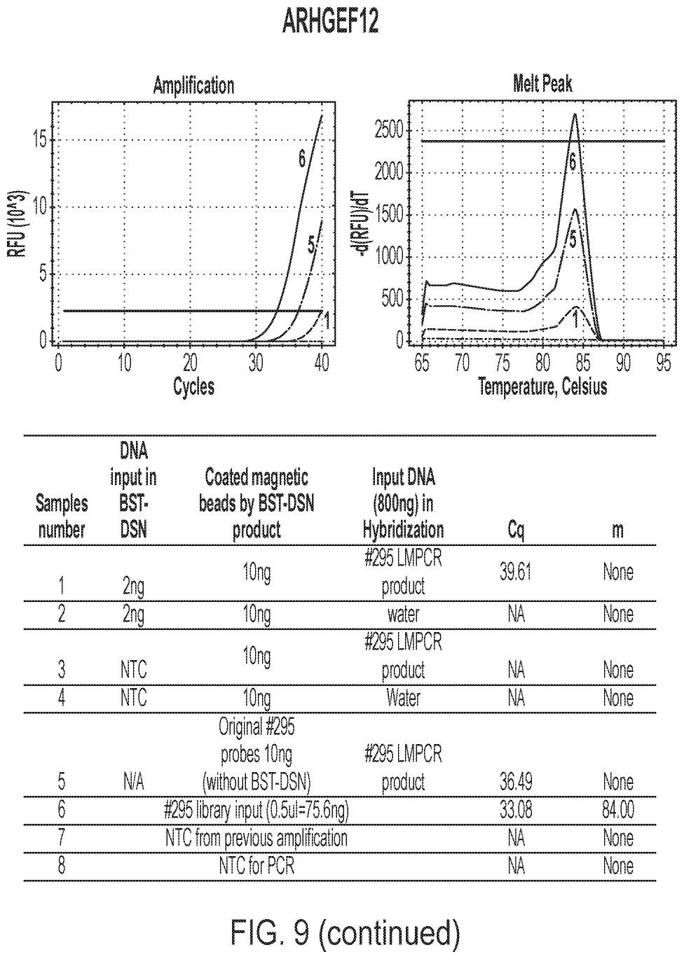

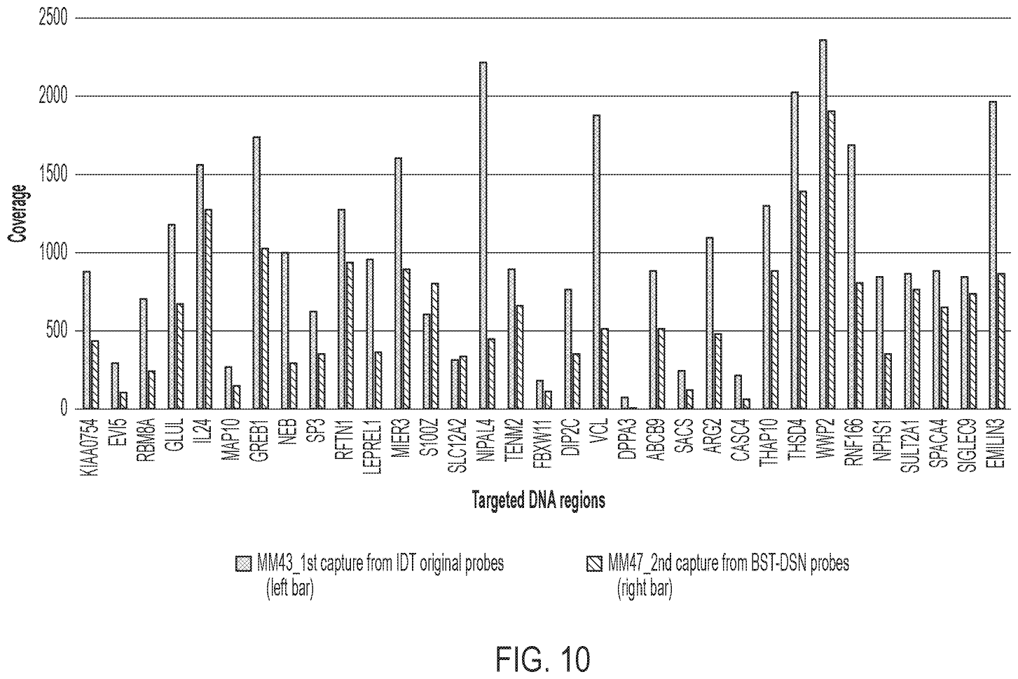

FIGS. 7-11 demonstrate further results for simultaneous capture of 33 DNA targets from patient DNA. To generate the Bst-DSN probes, 33 single stranded commercially synthesized probes (‘Ultramers’ from IDT) corresponding to genomic targets mutated in the primary tumor of an individual were used. FIGS. 7-11 demonstrate the generation of amplified biotinylated probes from these 33 Ultramers, which can then be used in hybrid capture reactions to enrich for the same 33 targets when hybridized with the human genomic DNA amplified from the blood of the same individual (circulating DNA as the starting material). The workflow followed is illustrated in FIG. 7, while FIGS. 8-10 demonstrate real-time PCR based validation for the capture specificity by measuring on- and off-targets that were captured by the probes.

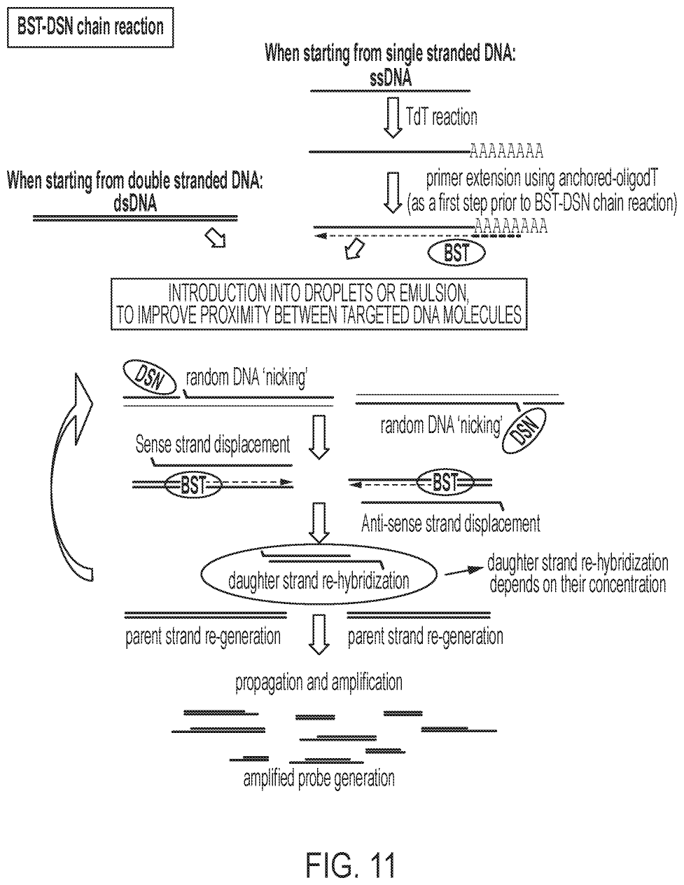

FIG. 11 compares sequencing results obtained after using commercially available capture probes and results obtained after using the Bst-DSN-generated probes. The results of the commercial probes are similar to the results of the Bst-DSN probes. However, the Bst-DSN probes are the result of amplification from the original probes and contain at least 100 times more DNA, thereby enabling at least 100 times more DNA target capture reactions. Accordingly, the cost per hybrid-capture reaction decreases by a factor of at least 100.

### Example 2: Generation of dsDNA Probes Using a Bst-DSN Chain Reaction, Validation of Probes; and Use of the Generated Probes to Enrich Target Sequences

In this Example, an isothermal DNA amplification reaction, BST-DSN is presented using two thermostable enzymes, BST DNA polymerase (BST) and duplex-specific nuclease (DSN) which has minimal activity on single stranded DNA (4,5) while it generates single strand breaks on double stranded DNA with no apparent sequence preference (4).

When dsDNA is applied as starting material in a BST-DSN reaction, DSN produces random single strand breaks (nicks, FIG. 1). The nicks are then recognized by BST which initiates strand displacement DNA synthesis and re-generates the original dsDNA molecule. The displaced DNA may re-hybridize with displaced DNA from an opposite strand of the DNA target and forms a daughter dsDNA which participates in new BST-DSN reactions. The BST-DSN chain reaction produces short DNA fragments from dsDNA template and reaches completion within minutes.

When ssDNA is applied as starting material in BST-DSN reaction, an additional step is included to convert ssDNA to dsDNA (FIG. 1). Terminal deoxynucleotidyl transferase (TdT) is employed to synthesize a poly-A tail on the 3′end of ssDNA. Products from TdT reaction are then used in BST-DSN reactions that include an anchor-oligo-dT to generate dsDNA from the poly-A tails, following which the chain amplification by BST-DSN takes place. By including biotinylated nucleotides in the TdT-BST-DSN (TBD) reaction, the amplification reaction produces copious amounts of biotinylated probes that can be used directly as ‘baits’ for target enrichment from human genomic DNA; thereby greatly increasing the number of reactions that can be performed from an initial input of capture probes and reducing the overall sample preparation cost. The TBD reaction-generated capture probes were validated using either a custom-made panel of PCR products as input DNA, or commercially available sets of long oligonucleotides (‘ultramers’) covering 190 or 7,816 genomic targets of interest, and by performing target enrichment and sequencing from amplified cell-free circulating DNA.

### Materials and Methods

### Cell-Free Circulating DNA (cfDNA) and Ligation-Mediated PCR (LMPCR)

cfDNA from healthy volunteers were obtained from Brigham and Women's Hospital and the Dana Farber Cancer Institute under Institutional Review Board approval. cfDNA was isolated from plasma using the QIAamp Circulating Nucleic Acids Kit (Qiagen) and was quantified on a Qubit 3.0 fluorometer using a dsDNA HS assay kit (Thermo Fisher Scientific). cfDNA was then subjected to end-repair and adaptor ligation (NEBNext Ultra II DNA Library Prep Kit, New England Biolabs, NEB) followed by 15 cycles of amplification via ligation-mediated PCR (LMPCR) using Q5 DNA polymerase (NEB).

LMPCR product similarly obtained by using cfDNA from cancer patient #295 was also used for this study, under Institutional Review Board approval. Somatic mutations in this sample had been previously identified via exome sequencing of the primary tumor, as well as via exome sequencing of cfDNA (6,7). To generate low mutation allelic frequency from this sample, a 20-fold dilution into LMPCR product obtained from healthy volunteers' cfDNA was applied.

### Polymerase Chain Reaction (PCR) Amplification of Gene Targets

PCR reactions targeting p53, NOP14, MTMR4, ZPLD1, CDHR3, GMPR, CACNA1I, OR2S2, AGHGEF12, CACNA1C, SAMDA4, KRAS, BRAF and NGLY1 were performed on CFX Connect™ real-time PCR machine (Bio-Rad Laboratories) using Phusion High-Fidelity DNA polymerase (Thermo Fisher Scientific) per the protocol provided in Table 3. All primers were synthesized by IDT (Integrated DNA Technologies, IDT) using primers depicted in Table 4.

TABLE 3
PCR protocols
PCR reaction for target specific amplification from human genome

| | | Final | | | | | |
|---|---|---|---|---|---|---|---|
| | 25 μl | concentration/ | | | | | |
| Reaction setup | Rxn (μl) | amount | | Thermo cycling | | | |
| 5 X Phusion HiFi buffer | 5 | 1 X | | 98 C. | 2 | min | 40 cycles |
| dNTP (10 mM each) | 2 | 0.8 | mM | 98 C. | 10 | sec | |
| 10 X LCGreen | 2 | 0.8 X | | 65 C. | 30 | sec | |
| 10 uM primer (F1 + R1) from Table 3 | 0.5 | 200 | nM | 72 C. | 30 | sec | |
| Phusion polymerase | 0.2 | 0.2 | U | 72 C. | 5 | min | |

| | | | | |
|---|---|---|---|---|
| 5 ng Human male genomic DNA | 1 | 5 | ng | Melting (optional) |

| | |
|---|---|
| dH2O | 14.3 |

Post-capture PCR for LMPCR products

| | | Final | | | | | | |
|---|---|---|---|---|---|---|---|---|
| | 25 μl | concentration/ | | | | | | |
| Reaction setup | Rxn (μl) | amount | | Thermo cycling | | | | |
| Q5 master mix | 25 | 1X | | 98 | C. | 30 | sec | 14 cycles |
| Illumina adaptor primer (F1 + R1) (10 uM) | 5 | 1 | uM | 98 | C. | 15 | sec | |
| MgCl2 (25 mM) | 1 | 0.5 | mM | 65 | C. | 30 | sec | |
| | | | | 72 | C. | 30 | sec | |
| | | | | 72 | C. | 1 | min | |

| | | |
|---|---|---|
| 4 | C. | Hold |

Two-step PCR
a.) LMPCR

| | | Final | | | | | |
|---|---|---|---|---|---|---|---|
| | 50 μl | concentration/ | | | | | |
| Reaction setup | Rxn (μl) | amount | | Thermo cycling | | | |
| 5 X Phusion HiFi buffer | 5 | 1 X | | 98 C. | 2 | min | 15 cycles |
| dNTP (10 mM each) | 2 | 0.8 | mM | 98 C. | 10 | sec | |
| 10 X LCGreen | 2 | 0.8 X | | 60 C. | 30 | sec | |
| Illumina adaptor primer (F1 + R1) (10 uM) | 0.5 | 200 | nM | 72 C. | 30 | sec | |
| Phusion polymerase | 0.2 | 0.2 | U | 72 C. | 5 | min | |

| Captured DNA | 10 | Melting (optional) |
|---|---|---|
| dH2O | 4.3 | |

b.) Target specific PCR

| | | Final | | | | | |
|---|---|---|---|---|---|---|---|
| | 25 μl | concentration/ | | | | | |
| Reaction setup | Rxn (μl) | amount | | Thermo cycling | | | |
| 5 X Phusion HiFi buffer | 5 | 1 X | | 98 C. | 2 | min | 40 cycles |
| dNTP (10 mM each) | 2 | 0.8 | mM | 98 C. | 10 | sec | |
| 10 X LCGreen | 2 | 0.8 X | | 65 C. | 30 | sec | |
| 10 uM specific primer (F1 + R1) from supplementary Table 1 | 0.5 | 200 | nM | 72 C. | 30 | sec | |
| Phusion polymerase | 0.2 | 0.2 | U | 72 C. | 5 | min | |

| 1:500 diluted product from LMPCR | 1 | Melting (optional) |
|---|---|---|
| dH2O | 14.3 | |

TABLE 4
Primers for PCT Reactions

| SEQ | | | |
|---|---|---|---|
| ID | Primer | | |
| NO: | ID | Sequence | Functions |
| 1 | NOP14-F1 | GCAGATGTGAGG | specific target |
| | | TAATGTCCAG | for 10 plex |
| 2 | NOP14-R1 | TGCGGAAAGAAA | capture |
| | | GCGGAAAG | |
| 3 | MTMR4-F1 | TGCTTCTGAAAG | specific target |
| | | GCAGTCTTC | for 10 plex |
| 4 | MTMR4-R1 | AGCGTTAATGAT | capture |
| | | GATGAAGATAAT | |
| | | TTG | |
| 5 | ZPLD1-F1 | CTGTGAGAGAGA | specific target |
| | | ACAATGGC | for 10 plex |
| 6 | ZPLD1-R1 | AGCCATTCACAT | capture |
| | | AAATGACTCCT | |
| 7 | CDHR3-F1 | GGTCTGGGTTCC | specific target |
| | | AGAAATGAG | for 10 plex |
| 8 | CDHR3-R1 | AGCATCTCAGTG | capture |
| | | ATTGACAGG | |
| 9 | GMPR-F1 | GACGGAAGCTCA | specific target |
| | | AGCTCTTC | for 10 plex |
| 10 | GMPR-R1 | CCCATCTCCGTA | capture |
| | | AGAAAAGCA | |
| 11 | CACNA1I-F1 | CCATTGTGCTAC | specific target |
| | | TGTCAGTCAT | for 10 plex |
| 12 | CACNA1I-R1 | CCCGTTCCCTGT | capture |
| | | CCCAC | |
| 13 | OR2S2-F1 | GGATGAGTTTGT | specific target |
| | | CTGAAAGATCCT | for 10 plex |
| 14 | OR2S2-R1 | AGGGGAGGAAAA | capture |
| | | AGGTCTTC | |
| 15 | ARHGEF12-F1 | TGTCTTTTCCTT | specific target |
| | | TCTCTGTTTTCT | for 10 plex |
| | | CT | capture |
| 16 | ARHGEF12-R1 | ACTGTACGAAGA | |
| | | CTGGATTGTC | |
| 17 | CACNA1C-F1 | TGGCTTTGCAGG | specific target |
| | | TCGAAGG | for 10 plex |
| 18 | CACNA1C-R1 | GTGAGCTACCTG | capture |
| | | ATGATGAACC | |
| 19 | SAMD4A-F1 | GCCACTTCGTTA | specific target |
| | | GAAGACCG | for 10 plex |
| 20 | SAMD4A-R1 | TGGTGATAGTAG | capture |
| | | TGTGTCTGTCC | |
| 21 | NGLY1-F1 | GCTGTATCAGAT | non-specific |
| | | CGCAATTTCC | target for |
| 22 | NGLY1-R1 | CATCTTTTGCTT | 10 plex |
| | | ATATTTCCTGGA | capture |
| | | AGT | |
| 23 | KRAS-F1 | ATTTCAGTGTTA | non-specific |
| | | CTTACCTGTCTT | target for |
| | | G | 10 plex |
| 24 | KRAS-R1 | ACAGGCTCAGGA | capture |
| | | CTTAGCAA | |
| 25 | BRAF-F1 | ACCACATTACAT | non-specific |
| | | ACTTACCATGCC | target for |
| 26 | BRAF-R1 | TGGGCAGATTAC | 10 plex |
| | | AGTGGGAC | capture |
| 27 | p53-Ex8-F1 | GAACAGCTTTGA | Single |
| | | GGTGGGTGTTT | target PCR |
| 28 | p53-Ex8-R1 | TGGTCTCCTCCA | |
| | | CCGCTTC | |
| 29 | Illumina | AC ACT CTT | LMPCR |
| | adaptor | TCC CTA CAC | |
| | primer-F1 | GAC CT CTT | |
| | | CCG ATCT | |
| 30 | Illumina | GACTGGAGTTCA | |
| | adaptor | GACGTGTCTCTT | |
| | primer-R1 | CCGATCT | |

### BST-DSN Reaction Using PCR Products as Input

BST 2.0 DNA polymerase (BST) and duplex-specific nuclease (DSN) were purchased from NEB and Sapphire North America, respectively. BST-DSN reaction was conducted. PCR products were mixed in a 10 μl final volume of BST-DSN reaction master mix per the protocol provided on Table 5. BST-DSN reaction was conducted in a Cepheid Smart cycler II thermocycler set at a constant 65° C. as shown on Table 5. The reaction was followed in real time by including a DNA intercalating dye, LCGreen (BioFire Diagnostics) in the reaction and reading the fluorescent signal in 12 second ‘cycles’. A QIAquick™ Nucleotide Removal Kit (Qiagen) was used to purify the BST-DSN products and the size of BST-DSN products was analyzed by Agilent DNA Chip 1000 (Agilent).

### BST-DSN Reaction Using Single Strand DNA (ssDNA) as Input

Custom long oligonucleotide ‘ultramer’ probes (33-plex, Panel A) were obtained from IDT (Integrated DNA Technologies). NEBNext Direct™ Cancer hotspot panel (Panel B) and xGen Pan Cancer™ panel (Panel C) were purchased from NEB and IDT, respectively. Terminal deoxynucleotidyl transferase (TdT, from NEB) reaction was performed on a thermocycler (Mastercycler Nexus, Eppendorf) to generate poly-adenines at the 3′end of ssDNA prior to BST-DSN reaction. The protocol is described in Table 5. The products generated from TdT reaction were then employed in a 10 μl final volume of BST-DSN reaction master mix containing Biotin-11-dUTP (B-dUTP) and anchored-oligo-dT (Table 5). Nucleotide removal kit (Qiagen) was used to purify the TBD products and the size of TBD products was analyzed on an Agilent DNA Chip 1000 analyzer (Agilent). Reactions were repeated at least three times to check the repeatability of results.

### Hybridization Capture

On-Bead Hybridization Capture

At first, capture using BST-DSN probes generated from PCR products was examined employing an on-bead hybridization capture procedure as described by Maricic et al (3). Briefly, 800 ng of BST-DSN probes generated from a mix of 10 PCR products were denatured at 98° C. for 2 min and immobilized on Dynabeads™ magnetic beads (Thermo Fisher Scientific) at room temperature for 15 min. Immobilized beads were then washed three times with 1× BWT (1 M of NaCl, 5 mM of Tris-Cl, pH8.0, 0.5 mM of EDTA, pH8.0) and 0.05% of Tween-20 buffer and re-suspended with 500 ng denatured LMPCR products in 1× oligonucleotide buffer, following by incubation at 65° C. for 16 hours. After washing one time with BWT buffer and two times with 1× Phusion buffer, the captured DNA was released in 1× Phusion buffer by incubation at 95° C. for 2 min. Target-specific capture was then validated by two-step PCR (FIG. 12).

### In-Solution Hybridization Capture Using IDT Hybridization and Wash Kit

In-solution hybridization capture was performed using xGen hybridization and wash kit according to the IDT protocol to examine the sequence-capture ability of TBD probes generated from Panel A/B/C. The NTC TBD, which is a No Template Control experiment where all steps are included in the absence of input DNA, was used as a negative control in capture. Briefly, LMPCR products (500 ng for the first capture and 200 ng for the second capture) and 7.5 μl of Human Cot DNA were concentrated by 1.8× AMPure XP beads (Beckman Coulter) and re-suspended in 1× hybridization buffer with hybridization enhancer, blocking oligos and probes prior to incubation at specific hybridization temperature for 16 hours. The hybrids formed between LMPCR targets and biotinylated probes were immobilized on Streptavidin beads at hybridization temperature for 45 min. Heat-wash was then applied at hybridization temperature with 1× Stringent Wash Buffer for 5 min, following by washing with wash buffer 1, 2 and 3 at room temperature. The captured DNA was then re-suspended in 20 μl Nuclease-Free water. Post-capture PCR was performed in a final volume of 50 μl Q5 master mix (NEB) with 1 μmol/L of each Illumina adaptor primer respectively. Post-capture PCR was performed as description on Table 3, following by purification using 1.8× AMPure beads (Beckman Coulter). The purified DNA was then eluted in 20 μl of 1× low-EDTA TE buffer (Quality Biological). Only one round of capture was performed for the original (commercial) probes, while either one or two rounds of capture was performed for TBD probes. The hybridization was performed at 65° C. or at 60° C. for the original commercial probes, per manufacturer's specifications. The hybridization temperature for TBD probes generated from Panel A was performed at 50° C. or 60° C. for the first capture, and 60° C. for the second capture. For TBD probes generated from Panel B/C, 50° C. was used for the hybridization in both first and second capture (FIG. 13)

### Two-Step PCR Validation after Capture

Two-step PCR was initially used to validate the presence of discrete DNA targets following target enrichment. The captured DNA was first amplified on CFX Connect™ real-time PCR machine (Bio-Rad Laboratories) using Illumina adaptor primers in a final volume of 25 μl using Phusion polymerase master mix per the protocol provided on Table 3. After amplification using Illumina adaptor primers, DNA was diluted 500× in Nuclease-Free water. Two μl of diluted DNA was employed as template for target specific PCR Prep, Table 3.

### Illumina MiSeq Sequencing and Data Analysis

The captured DNA underwent library preparation using NEBNext Ultra II DNA Library Kit for Illumina sequencing (NEB). Samples were quality and quantity tested via Agilent Bio-analyzer and KAPA Library Amplification Kit (KAPA Biosystems), and then pooled in a single tube prior to Illumina MiSeq Sequencing at the Molecular Biology Core Facility, Dana-Farber Cancer Institute. Human genome hg19 was employed as template for alignment prior to data analysis conducted via ngsCAT software tool (8) and Picard (http://broadinstitute.github.io/picard/) analysis.

### Results

### BST-DSN Reaction

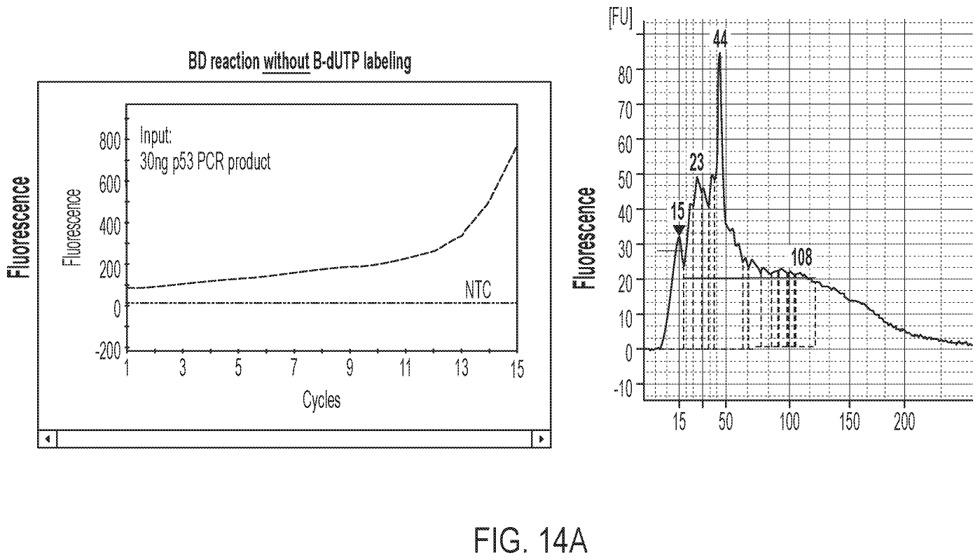

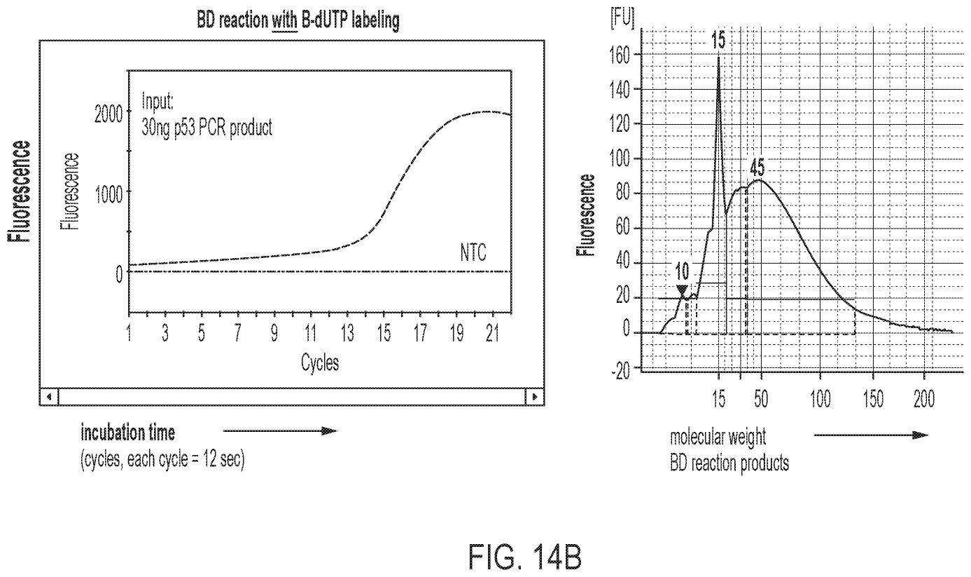

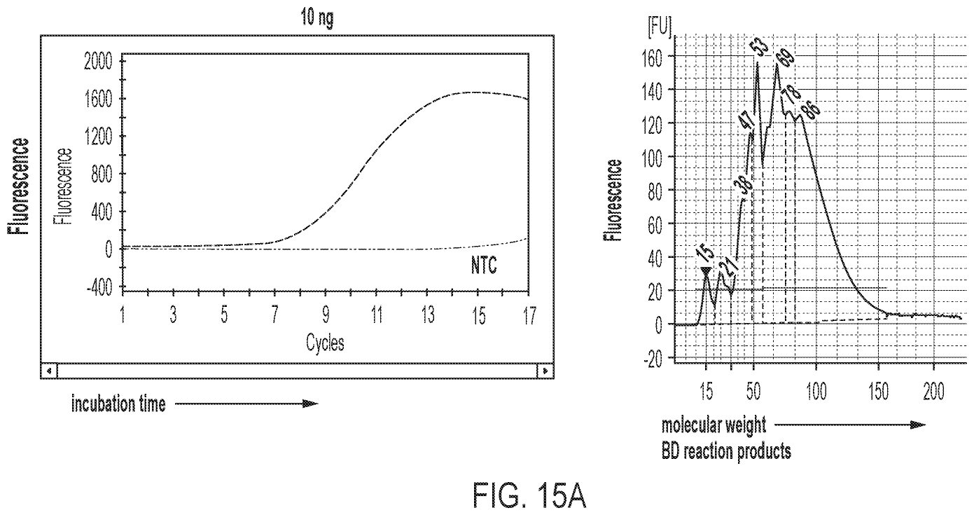

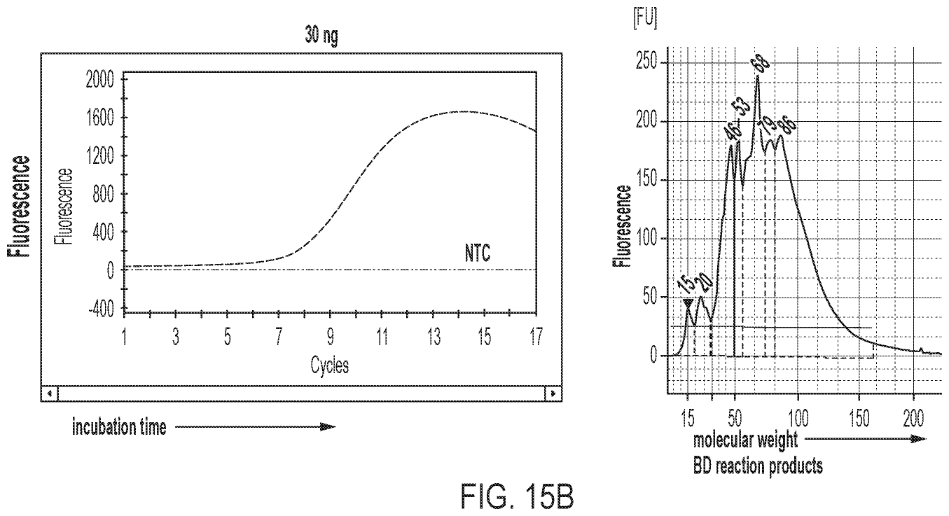

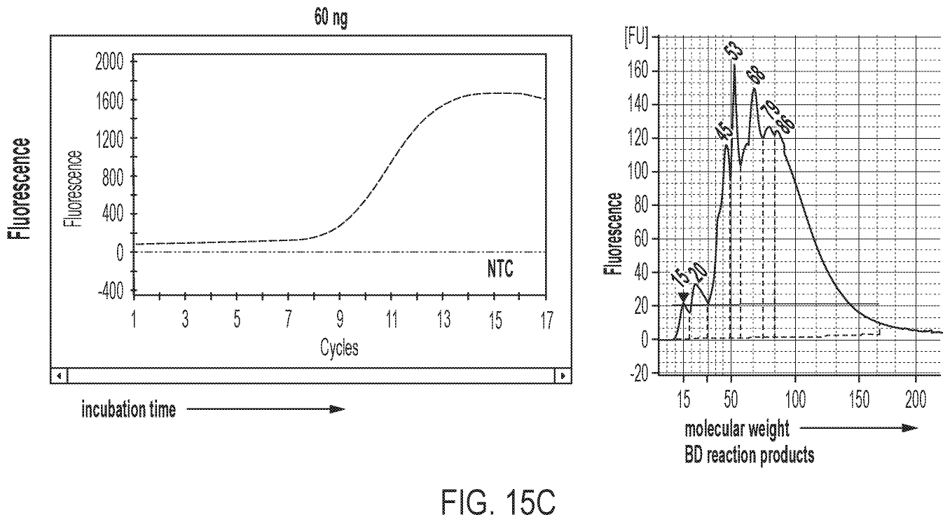

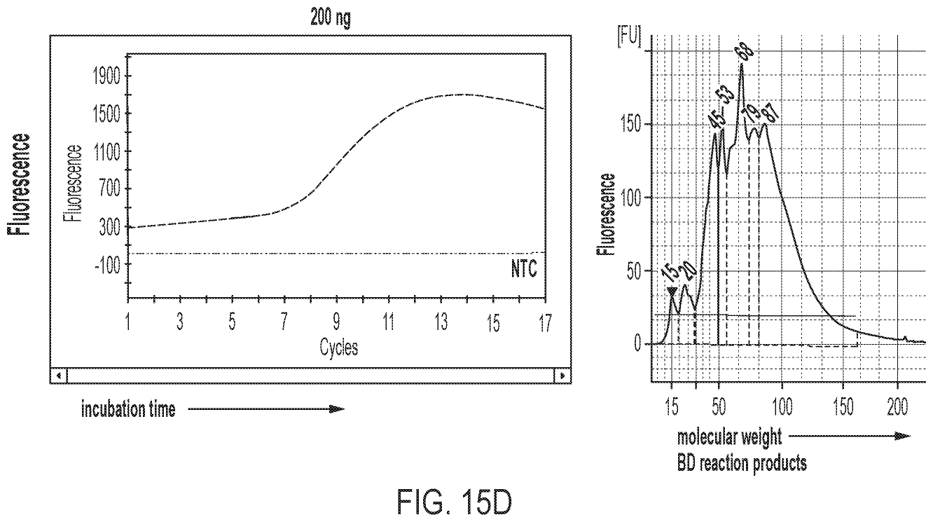

BST-DSN reaction with dsDNA (PCR products): The BST-DSN reaction (FIG. 1) was applied using a single PCR product as input (p53 exon 8, 157 bp amplicon) and the reaction was monitored in real time using a DNA intercalating dye (FIG. 14A-14B). Following completion of the reaction, the size range of BST-DSN products was measured via electrophoresis. The BST-DSN reaction, with or without B-dUTP labelling, amplified linearly at the beginning and entered an exponential phase after ˜2.5 min incubation at 65° C. Most BST-DSN products from a single PCR amplicon were in a size range of 20-80 bp (FIG. 14A-14B). Next, a mix of 10 PCR products, containing varying amounts of 10-200 ng total DNA, was used as input in the presence of B-dUTP. The amplification of the 10 PCR products reached plateau after ˜3 min incubation (FIG. 15A-15D). Moreover, BST-DSN products 20-80 bp size were generated irrespective of DNA input amount under the conditions applied, while the full range of products was 15-150 bp.

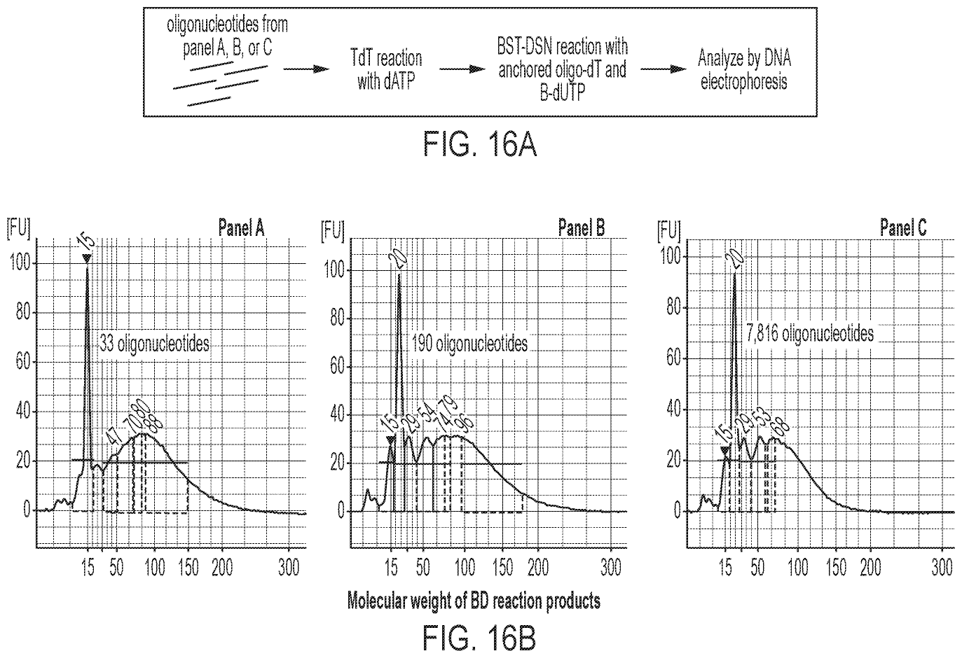

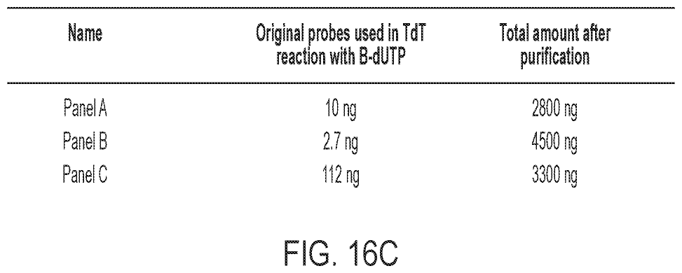

BST-DSN reaction with ssDNA (synthetic oligonucleotides): Single stranded oligonucleotides (107-2313 bp long) were used with TdT and followed by BST-DSN reaction to produce amplified B-dUTP-labeled probes. The probe sets tested were a set of 33 target-specific 120 bp long oligonucleotides previously used in our laboratory for hybridization capture and target enrichment prior to sequencing (Panel A, ‘ultramers’ from IDT, 120 bp/probe); the NEBNext™ Direct hotspot cancer panel (Panel B, 190 targets covering 50 genes probes, 107-2313 bp/probe) and the xGen Pan Cancer panel (Panel C, 7,816 ultramer probes covering 127 genes, 120 bp/probe). Results showed that 2800 ng, 4500 ng and 3300 ng of TBD probes were generated from an initial 10, 2.7 and 112 ng of Panels A-C, respectively. The median probe size is 69 bp and the range of probe sizes is 20-120 bp (FIG. 16A-16C).

### Application of Biotinylated TBD Probes for Target Specific Capture

To test the application of biotinylated probes towards target-specific capture from amplified genomic DNA, the products of TBD reactions were used as part of DNA sample preparation protocol prior to sequencing. Circulating DNA obtained from normal volunteers was end-repaired, ligated to adaptors and amplified via ligation-mediated PCR (LMPCR). The LMPCR product was then used for target-specific enrichment using the biotinylated BST-DSN probes for target capture on streptavidin-coated magnetic beads.

### Target Capture Via Probes Generated Using a Mix of PCR Products

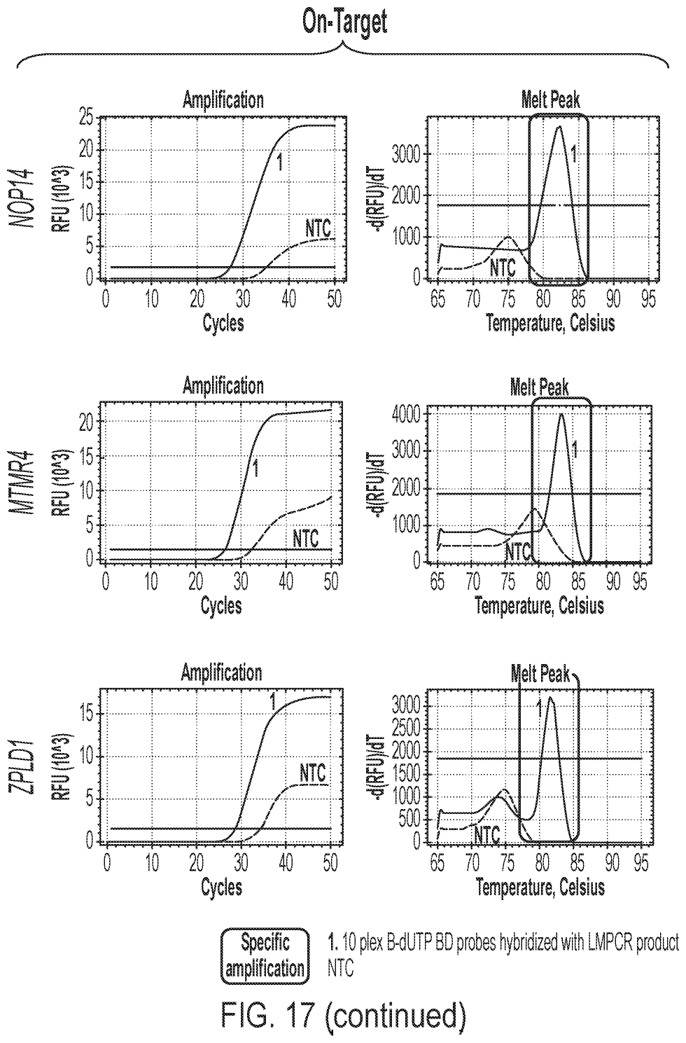

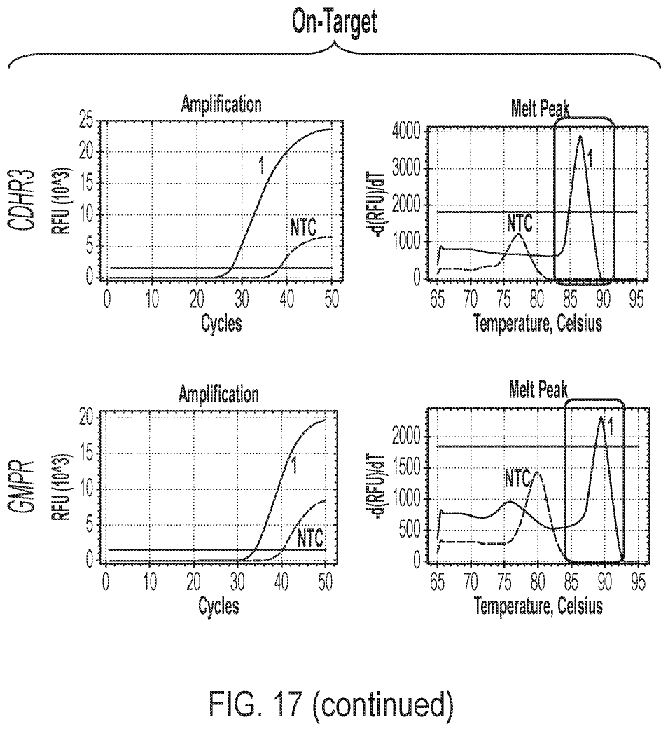

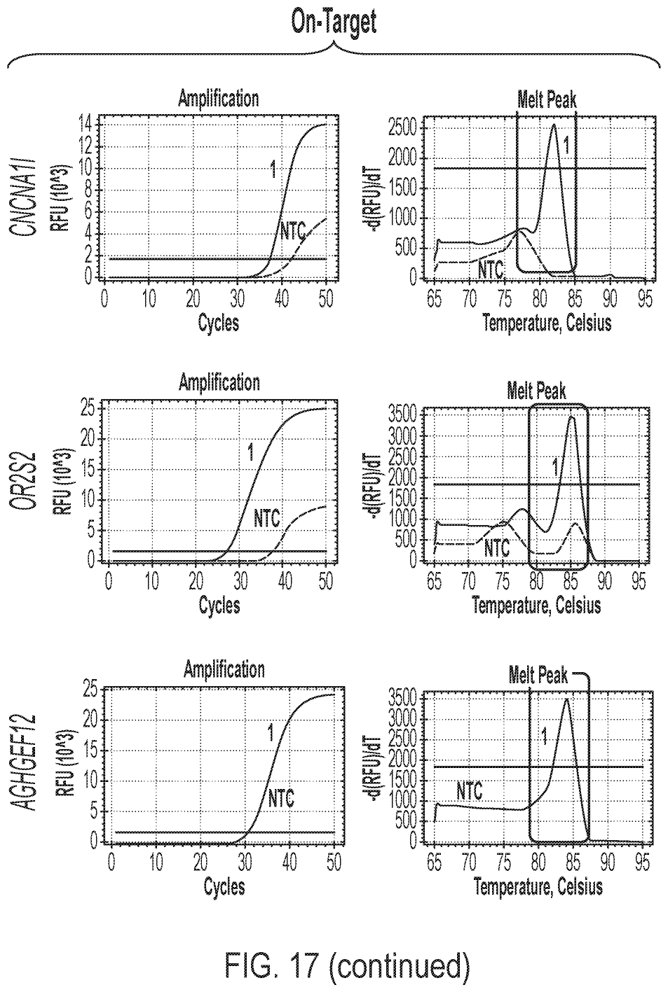

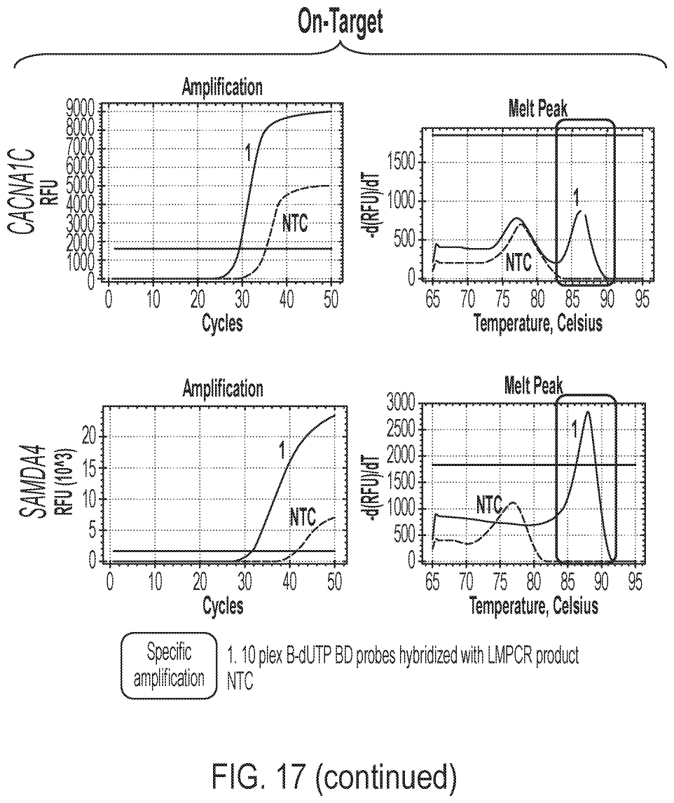

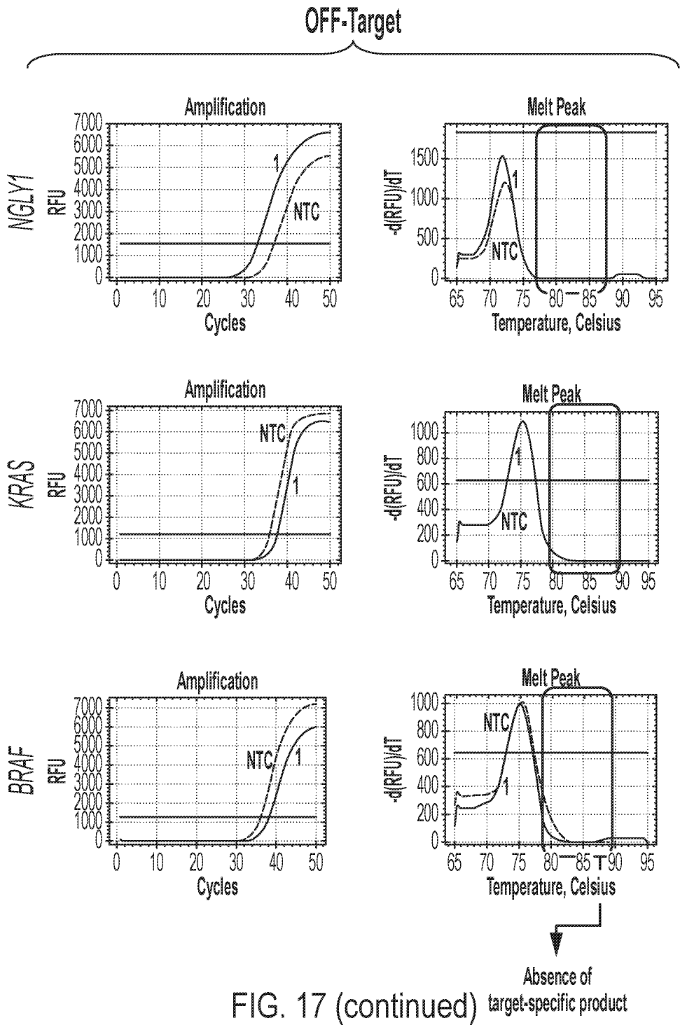

As a first test for hybrid capture, biotinylated BST-DSN probes generated from an equimolar mix of 10 PCR products were bound to streptavidin beads and then used to enrich the 10 specific targets from LMPCR product, according to the incubation protocol by Marici et al (3), FIG. 12. Following washing steps, ten specific targets (‘On-Target’) and three non-specific targets (Off-Target) were examined by target-specific PCR. All specific targets showed amplification from bead-bound DNA, while no amplification from the non-specific targets was observed (FIG. 17).

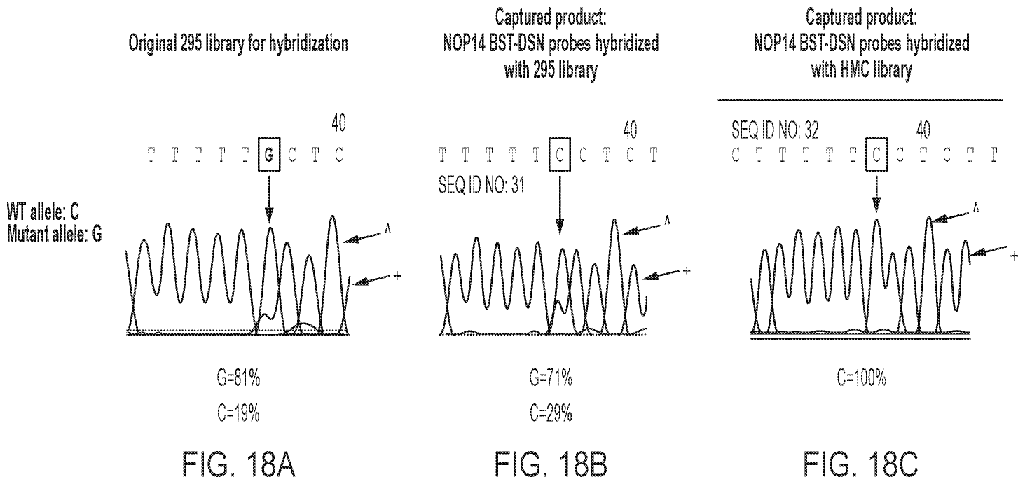

To examine whether BST-DSN probes capture both WT and mutant alleles from target DNA, BST-DSN capture probes corresponding to a mutation-containing target (NOP14 gene PCR product) were used. These were used for target capture using either HMC WT control LMPCR product or LMPCR product from DNA containing NOP14 mutation with mutation allelic frequency MAF of ˜81%. After capture, nested PCR followed by Sanger sequencing was applied. An MAF of 0% and 71% were observed using WT and mutation-containing LMPCR products, respectively (FIG. 18A-18C).

### Target Capture Using TBD Probes Generated from Panel A (33-Target Long-Oligonucleotide ‘Ultramer’ Mix)

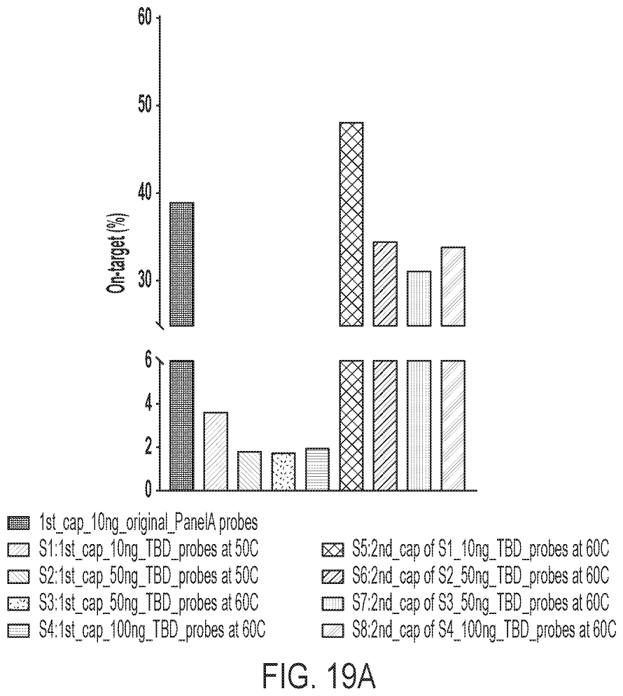

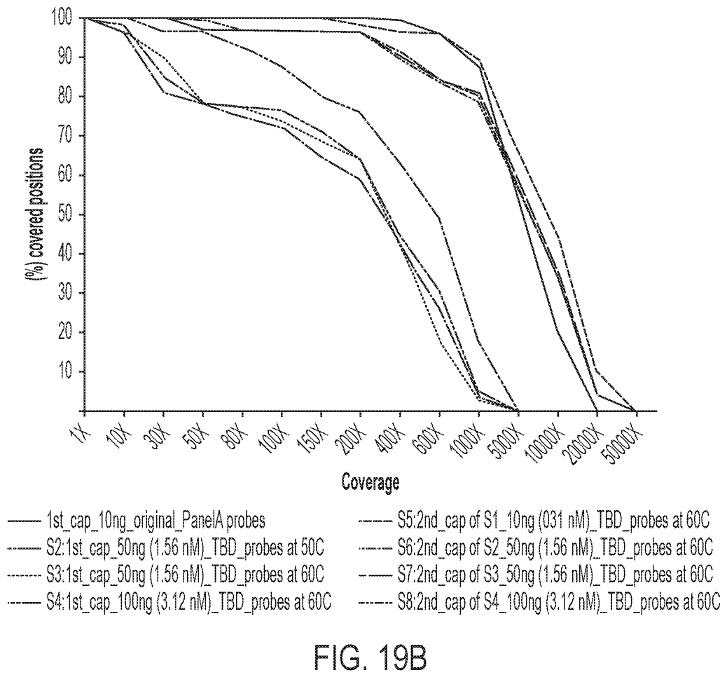

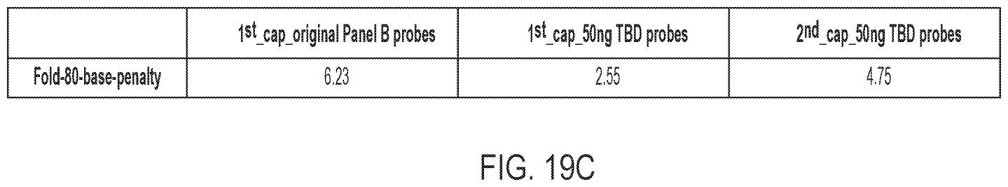

TBD probes generated from 33 biotin-labeled ultramers, Panel A, were used in capture reactions from LMPCR products, followed by sequencing. In this workflow (FIG. 13), biotinylated probes were first hybridized to the target DNA in solution and then bound to beads, as per manufacturer protocol. Either a single or two rounds of target-specific capture was applied to the biotinylated TBD probes. The amounts of TBD probes used as input in the hybridization reaction was varied to assess the impact on capture efficiency (Table 6). The captured DNA was analyzed by MiSeq-based sequencing and the capture ability was examined using on-target sequence ratio, coverage and uniformity using the ngsCAT tool (8). The data show that a single round of capture using TBD probes displays inferior on-target percentage and uniformity compared to a single round capture using the original commercial probes (FIG. 19A). Performing a second capture via TBD probes show that a second capture using TBD probes produces comparable results to a single round capture using the original probes (35-40% on-target ratio for either case). Similar conclusions apply to the uniformity of sequence coverage using the original probes vs. TBD-probes (FIG. 19B). The analysis of Fold-80-base-penalty also showed similar results, 2.55 and 4.75 on the 1^{st} and 2^{nd} round capture using TBD probes and 6.23 using original probes (FIG. 19C).

TABLE 6
Concentration of TBD probes used in capture

| | Concentration (total) | | Concentration (each) | |
|---|---|---|---|---|
| Panel A (33 targets) | | | | |
| 10 ng_TBD_probes | 10.3 | nM | 0.31 | nM |
| 50 ng_TBD_probes | 51.5 | nM | 1.56 | nM |
| 100 ng_TBD_probes | 103 | nM | 3.12 | nM |
| Panel B (190 targets) | | | | |
| 1 ng_TBD_probes | 4.53 | nM | 0.02 | nM |
| 5 ng_TBD_probes | 22.66 | nM | 0.12 | nM |
| 10 ng_TBD_probes | 45.32 | nM | 0.24 | nM |
| 50 ng_TBD_probes | 226.59 | nM | 1.19 | nM |
| Panel C (7816 targets) | | | | |
| 1 ng_TBD_probes | 4.53 | nM | 0.58 | pM |
| 5 ng_TBD_probes | 22.66 | nM | 2.9 | pM |
| 10 ng_TBD_probes | 45.32 | nM | 5.8 | pM |
| 50 ng_TBD_probes | 226.59 | nM | 29 | pM |
| 100 ng_TBD_probes | 453.19 | nM | 58 | pM |

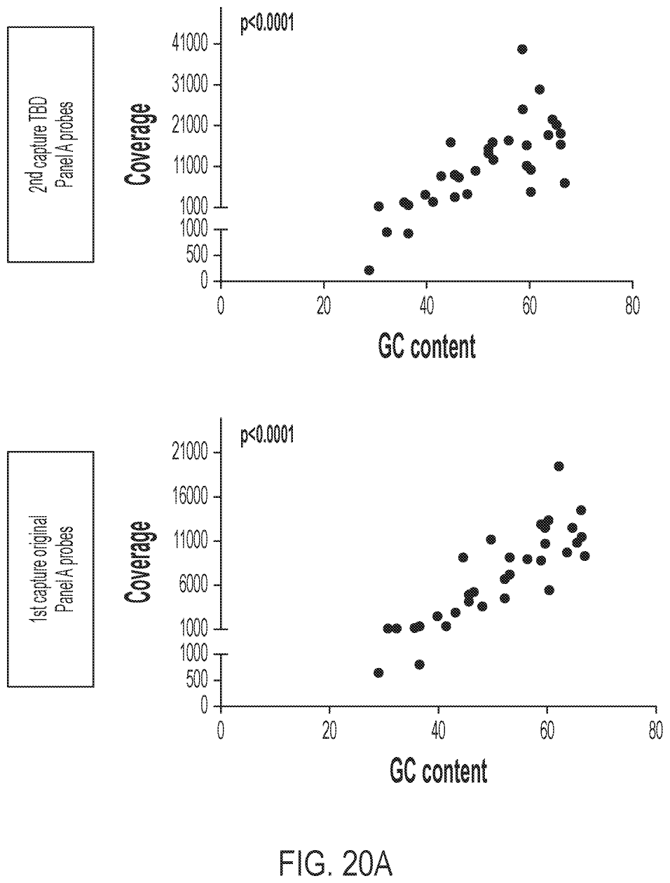

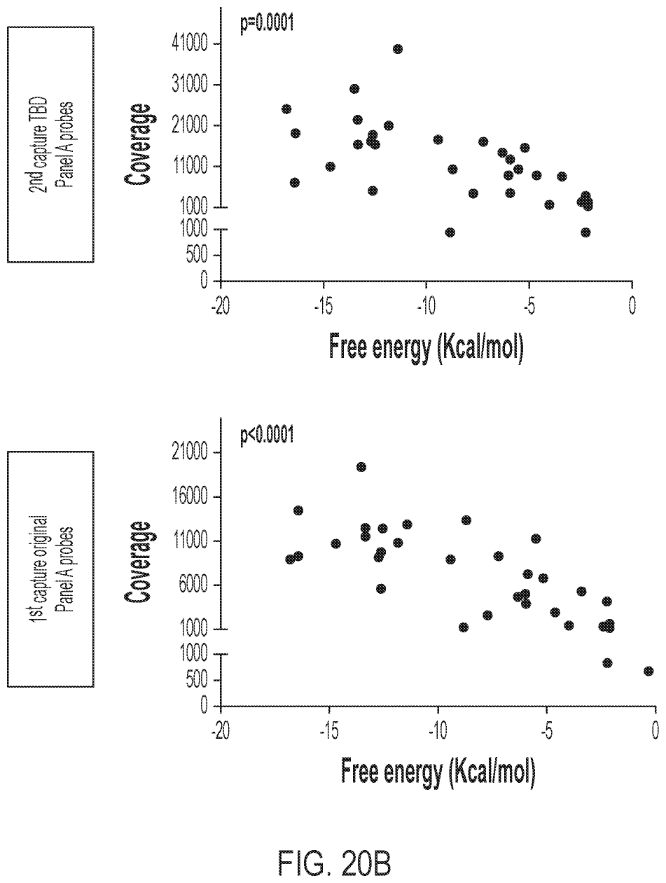

To investigate how GC content and secondary sequence structure affect hybridization capture, the second capture coverage using TBD probes generated from Panel A ultramers, as well the unmodified commercial ultramers were analyzed (FIG. 20A-20B). The data indicate a similar dependence of coverage and GC content/secondary structure and free energy for TBD probes and the unmodified ultramers. In both cases, coverage was found to decline with GC content.

The ability of TBD probes to recover targets containing low-level mutations was also investigated by applying the same protocol to a DNA sample from a cancer patient. LMPCR product from patient 295 was diluted 20-fold into WT LMPCR product obtained from a normal volunteer, to generate DNA containing panel A target mutations close to mutation allelic frequency MAF˜1%, which is at the detection limits of Miseq sequencing analysis (9). Table 7 shows that, most targets anticipated to harbor mutations at the ˜1% level are detected by both ultramer-based capture and TBD probe-based double capture. No mutations at these targets were detected when LMPCR product from normal volunteers was used (not shown).

TABLE 7
Application of original ultramer probes, or ultramer-derived
TBD probes to capture DNA targets with low-level mutations

| | | | | Panel A |
|---|---|---|---|---|
| | | | Panel A | TBD probe |
| DNA | | | ultramer probe | double |
| target | position | mutation | capture MAF | capture MAF |
| MAP10 | chr1:232942818 | C > G | 0.26% * | 1.18% |
| RBM8A | chr1:145507691 | G > A | 1.82% | 2.64% |
| EVI5 | chr1:93029240 | T > C | 1.18% | 1.78% |
| NPHS1 | chr19:36322234 | G > C | 1.50% | 0.33% * |
| SPACA4 | chr19:49110373 | C > G | 0.22% * | 1.22% |
| RBM8A | chr1:145507691 | G > A | 1.82% | 2.64% |
| GREB1 | chr2:11777848 | G > T | 1.12% | 0.51% * |

* Value near of below noise threshold of ~0.5%

### Random Pattern of BST-DSN Probe Generation

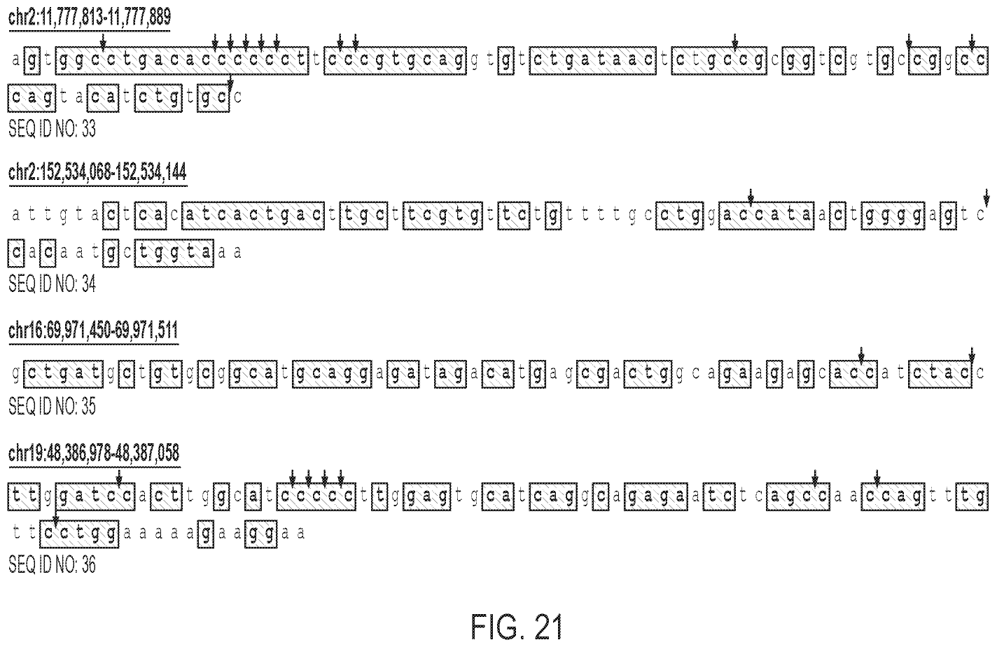

To investigate the distribution of DSN cutting positions during TBD probe generation, sequencing adaptors were ligated to TBD probes themselves and subjected the probes to Miseq sequencing. The first nucleotide in each sequencing read was then assumed to represent a DSN cutting site, since BST synthesis starts from this position. FIG. 21 depicts four representative sequences from panel A and indicates that DSN digestion is random, enabling BST to initiate synthesis at almost every sequence position.

### Target Capture Using TBD Probes Generated from Panel B (190 Target Commercial Oligonucleotide Panel) and from Panel C (7,816 Target Commercial Oligonucleotide Panel)

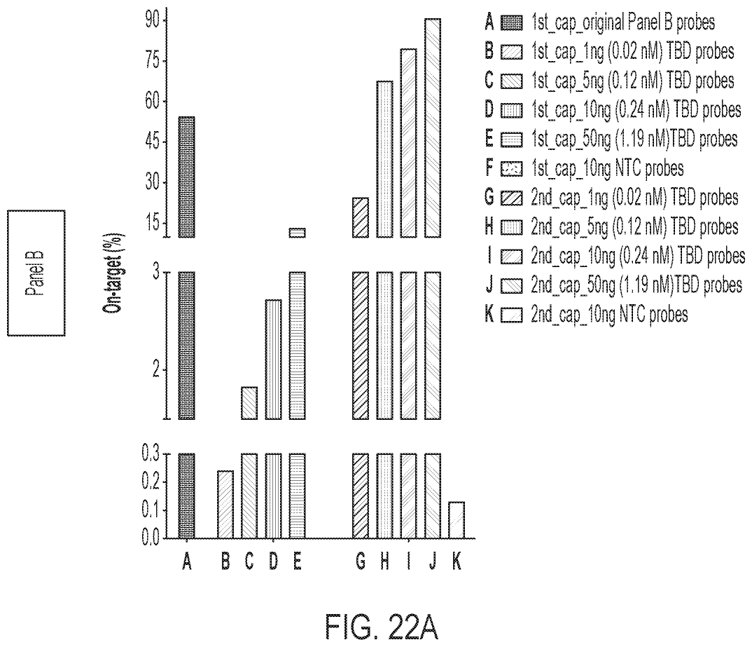

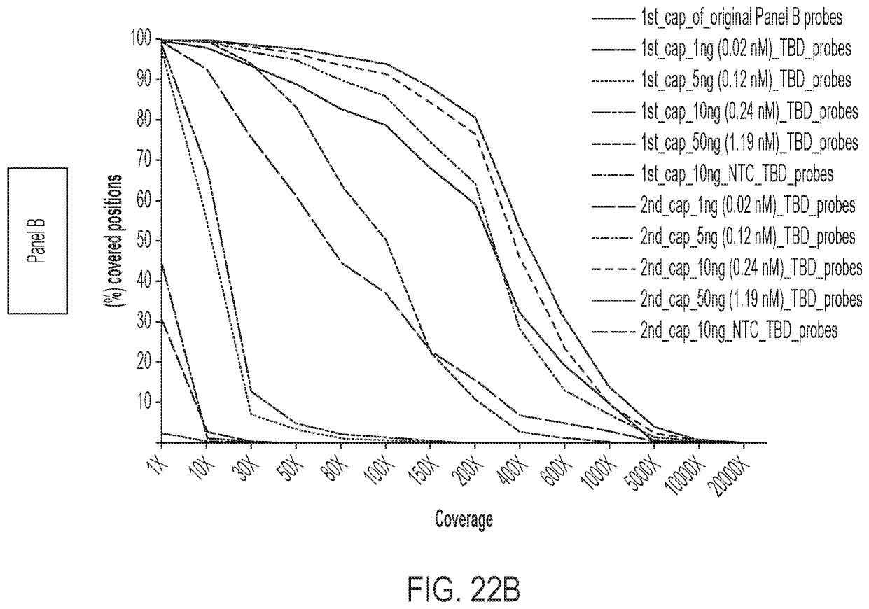

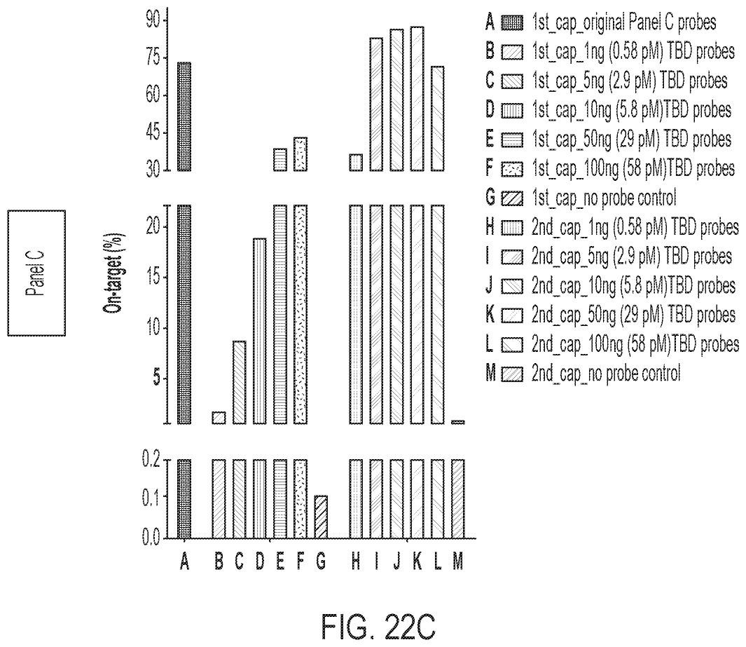

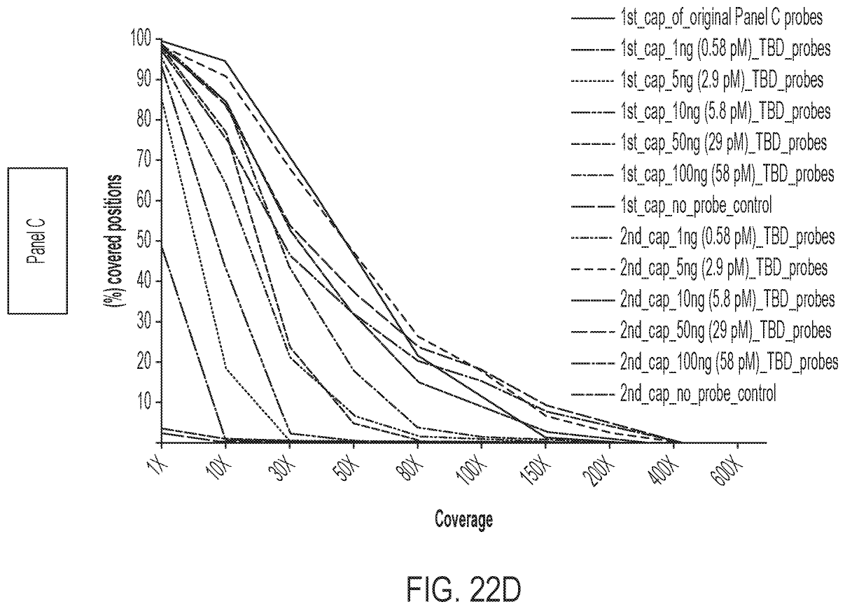

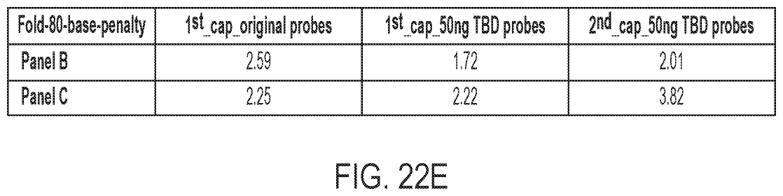

TBD probes generated using a 190 target cancer-specific panel (panel B, NEBNext™ Direct hotspot) was tested next, using the same workflow used for Panel A, and by varying the input 1-50 ng TBD probes during hybridization to LMPCR products. The results (FIG. 22A) show that a single round of capture using TBD probes from all conditions has inferior on-target ratio (1-15%) compared to a single round capture using the original, commercial probes (50%). However, a second round of capture by TBD probes using at least 5 ng BD probes as input results to a better on-target percentage, 60-90%, as compared to a single round of commercial probes, FIG. 22A. Similar conclusions apply to the coverage (FIG. 22B) and uniformity performance (FIG. 22E) as assessed by the fold-80-base penalty function.

Next, TBD probe-based capture generated using a 7,816 oligonucleotides comprehensive cancer panel C as input was evaluated using the same workflow as for panel B. Results (FIG. 22C) depict the same trend as those shown for panel B. A single round of capture using TBD probes at varying input for hybridization to LMPCR product, 1-100 ng, has somewhat inferior on-target ratio performance (3-45%) compared to a single round capture using the original, commercial probes (73%). In contrast, the second round of capture using TBD probes revealed superior on-target capture (82-88%) as compared to a single round via the commercial probes (73%), FIG. 22C. Moreover, a second round of capture using TBD probes showed comparable coverage (FIG. 22D) and uniformity (FIG. 22E) to the first capture of the commercial probes.

Finally, the on-target percentage for panels A-C was also assessed by including the target-flanking regions as part of target-specific capture. Minor improvement in target capture was seen in selected cases (Table 8) if the flanking regions are assumed to be part of the captured target.

TABLE 8
Comparison of the on-target captured percentage using
the probe regions with/without 300 bp flanking region

| samples | on-target (%) | on-target (%) with 300 bp flanking region |
|---|---|---|
| Panel A | | |
| 1st capture with 10 ng BD probes | 3.6 | 4.5 |
| 2nd capture with 10 ng BD probes | 48.1 | 60.1 |
| 1st capture with Panel A original probes | 38.9 | 46.8 |
| Panel B | | |
| 1st capture with 50 ng BD probes | 13.4 | 13.5 |
| 2nd capture with 50 ng BD probes | 90.7 | 91.0 |
| 1st capture with Panel B original probes | 54.2 | 54.9 |
| Panel C | | |
| 1st capture with 50 ng BD probes | 39.3 | 42.0 |
| 2nd capture with 50 ng BD probes | 87.5 | 93.7 |
| 1st capture with Panel C original probes | 73.3 | 79.8 |

While isothermal amplification reactions using endonucleases for genome-wide (10) or target-specific DNA amplification (11,12) have been described, the action of these enzymes relies on the presence of a DNA recognition sequence on the target DNA and is not random. As such, genomic regions with low-levels of recognition sequences might not amplify effectively. Nucleases, on the other hand, are not dependent on a recognition sequence (4) and can digest homopolymers efficiently (13). Thereby DSN would be expected to amplify and generate probes with good representation of any double stranded DNA template, independent of sequence. Indeed, the data in FIG. 21 show that DSN digests sequences at almost every position, thus initiating synthesis in a sequence-independent manner. In contrast, a CviPII ‘nickase’-based amplification as described by Chan et al (10) requires -CC- for digestion and would not produce random nicking on these sequences (FIG. 21, arrows). Accordingly, by replacing endonucleases with DSN nuclease in a BST-DSN chain reaction, capture probes likely representing all sections of an original sequence are generated. Consistent with this expectation, TBD amplification reactions produce capture probes leading to uniform capture of DNA targets from amplified genomic DNA.

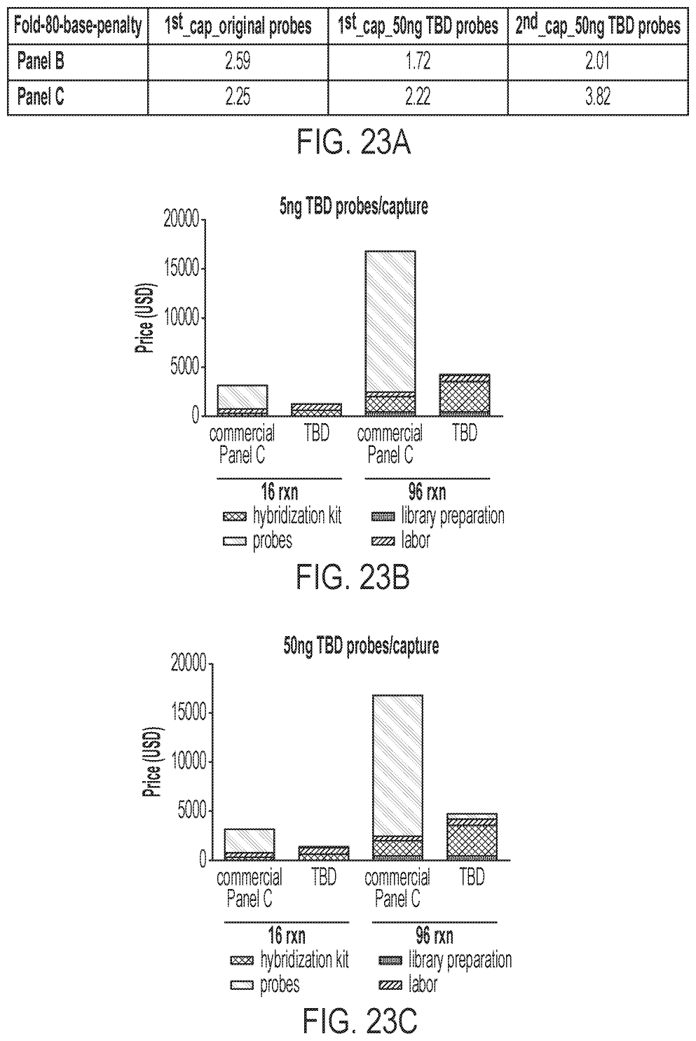

The data in FIG. 23A-23C indicate that by amplifying commercial panels of oligonucleotides prior to performing capture reactions enables major reagent savings. For example, using commercial probes suitable for a single capture reaction as the DNA input in a TBD reaction (Panel B, 190 targets) produces 4500 ng of TBD probes, which is enough for 45-450 double-capture reactions using biotinylated TBD probes (FIG. 23A). Also, using commercial probes appropriate for a single capture reaction as the DNA input in a TBD reaction (Panel C, 7,186 targets) produces 3300 ng of TBD probes, which is enough for 33-330 double-capture reactions using biotinylated TBD probes (FIG. 23A). TBD reactions are complete in minutes, while the overall process with purifications is less than 2 hours. Moreover, a two-round capture using TBD probes produces excellent (>80%) on-target ratio and uniformity (fold-80-base penalty). In effect, the reagent cost for target capture is diminished by following the present approach, albeit at the cost of introducing an additional capture step. This additional step increases labor cost, but as FIG. 23B and FIG. 23C show, the overall cost of sample preparation is reduced a lot. Reducing cost of sample preparation reagents lowers the overall cost of targeted re-sequencing. Additional reductions in overall re-sequencing cost can be achieved via mutation enrichment approaches which reduce the number of wild-type molecules that needs be sequenced (5,14). For example, PCR amplification using wild-type DNA-suppression approaches (15,16) have been shown to boost the mutant allelic fraction and reduce the amount of sequencing required to call mutations (17,18), in addition to increasing mutation detection threshold in conventional sequencing applications in cancer and in prenatal diagnostics (19,20).

Compared to an alternative way for amplification of commercial capture probes by synthesizing probes with two universal regions, then amplifying the universal regions with biotinylated primers, the TBD method has the advantage of amplifying any pre-existing set of probes without requiring sequence information and without presence/absence of universal regions. Most manufacturers currently do not provide information on universal regions hence TBD enables small laboratories to reduce cost on expensive capture probes irrespective of commercial format. Further, including universal regions in the probes may promote probe self-hybridization during capture unless a new ‘blocker’ oligonucleotide is used to prevent this. A disadvantage of the TBD method is that, under the current capture protocol, two sequential capture reactions are needed instead of one to achieve high ‘on-target’ fraction.

In summary, presented above a simple approach to amplify panels of double-stranded DNA or long oligonucleotide probes used for target enrichment prior to sequencing, using a novel nuclease-polymerase isothermal chain reaction and without knowledge of the sequences involved. This approach enables an increase in efficiency and reduction in cost of reagents used for sample preparation in targeted re-sequencing applications.

### REFERENCES TO EXAMPLE 2

1. Mamanova, L., Coffey, A. J., Scott, C. E., Kozarewa, I., Turner, E. H., Kumar, A., Howard, E., Shendure, J. and Turner, D. J. (2010) Target-enrichment strategies for next-generation sequencing. Nat Methods, 7, 111-118.

2. Mertes, F., Elsharawy, A., Sauer, S., van Helvoort, J. M., van der Zaag, P. J., Franke, A., Nilsson, M., Lehrach, H. and Brookes, A. J. (2011) Targeted enrichment of genomic DNA regions for next-generation sequencing. Brief Funct Genomics, 10, 374-386.

3. Maricic, T., Whitten, M. and Paabo, S. (2010) Multiplexed DNA sequence capture of mitochondrial genomes using PCR products. PLoS One, 5, e14004.

4. Shagin, D. A., Rebrikov, D. V., Kozhemyako, V. B., Altshuler, I. M., Shcheglov, A. S., Zhulidov, P. A., Bogdanova, E. A., Staroverov, D. B., Rasskazov, V. A. and Lukyanov, S. (2002) A novel method for SNP detection using a new duplex-specific nuclease from crab hepatopancreas. Genome Res, 12, 1935-1942.

5. Song, C., Liu, Y., Fontana, R., Makrigiorgos, A., Mamon, H., Kulke, M. H. and Makrigiorgos, G. M. (2016) Elimination of unaltered DNA in mixed clinical samples via nuclease-assisted minor-allele enrichment. Nucleic Acids Res, 44, e146.

6. Ladas, I., Fitarelli-Kiehl, M., Song, C., Adalsteinsson, V. A., Parsons, H. A., Lin, N. U., Wagle, N. and Makrigiorgos, G. M. (2017) Multiplexed Elimination of Wild-Type DNA and High-Resolution Melting Prior to Targeted Resequencing of Liquid Biopsies. Clin Chem, 63, 1605-1613.

7. Adalsteinsson, V. A., Ha, G., Freeman, S. S., Choudhury, A. D., Stover, D. G., Parsons, H. A., Gydush, G., Reed, S. C., Rotem, D., Rhoades, J. et al. (2017) Scalable whole-exome sequencing of cell-free DNA reveals high concordance with metastatic tumors. Nat Commun, 8, 1324.

8. Lopez-Domingo, F. J., Florido, J. P., Rueda, A., Dopazo, J. and Santoyo-Lopez, J. (2014) ngsCAT: a tool to assess the efficiency of targeted enrichment sequencing. Bioinformatics, 30, 1767-1768.

9. Mehrotra, M., Singh, R. R., Chen, W., Huang, R. S. P., Almohammedsalim, A. A., Barkoh, B. A., Simien, C. M., Hernandez, M., Behrens, C., Patel, K. P. et al. (2017) Study of Preanalytic and Analytic Variables for Clinical Next-Generation Sequencing of Circulating Cell-Free Nucleic Acid. J Mol Diagn, 19, 514-524.

10. Chan, S. H., Zhu, Z., Van Etten, J. L. and Xu, S. Y. (2004) Cloning of CviPII nicking and modification system from chlorella virus NYs-1 and application of Nt.CviPII in random DNA amplification. Nucleic Acids Res, 32, 6187-6199.

11. Joneja, A. and Huang, X. (2011) Linear nicking endonuclease-mediated strand-displacement DNA amplification. Anal Biochem, 414, 58-69.

12. Gill, P. and Ghaemi, A. (2008) Nucleic acid isothermal amplification technologies: a review. Nucleosides Nucleotides Nucleic Acids, 27, 224-243.

13. Ladas, I., Yu, F., Leong, K. W., Fitarelli-Kiehl, M., Song, C., Ashtaputre, R., Kulke, M., Mamon, H. and Makrigiorgos, G. M. (2018) Enhanced detection of microsatellite instability using pre-PCR elimination of wild-type DNA homo-polymers in tissue and liquid biopsies. Nucleic Acids Res, 46, e74.

14. Li, J., Wang, L., Mamon, H., Kulke, M. H., Berbeco, R. and Makrigiorgos, G. M. (2008) Replacing PCR with COLD-PCR enriches variant DNA sequences and redefines the sensitivity of genetic testing. Nat Med, 14, 579-584.

15. Milbury, C. A., Li, J. and Makrigiorgos, G. M. (2009) PCR-based methods for the enrichment of minority alleles and mutations. Clin Chem, 55, 632-640.

16. Milbury, C. A., Li, J. and Makrigiorgos, G. M. (2011) Ice-COLD-PCR enables rapid amplification and robust enrichment for low-abundance unknown DNA mutations. Nucleic Acids Res, 39, e2.

17. Wu, L. R., Chen, S. X., Wu, Y., Patel, A. A. and Zhang, D. Y. (2017) Multiplexed enrichment of rare DNA variants via sequence-selective and temperature-robust amplification. Nat Biomed Eng, 1, 714-723.

18. Milbury, C. A., Correll, M., Quackenbush, J., Rubio, R. and Makrigiorgos, G. M. (2012) COLD-PCR enrichment of rare cancer mutations prior to targeted amplicon resequencing. Clin Chem, 58, 580-589.

19. Murphy, D. M., Bejar, R., Stevenson, K., Neuberg, D., Shi, Y., Cubrich, C., Richardson, K., Eastlake, P., Garcia-Manero, G., Kantarjian, H. et al. (2013) NRAS mutations with low allele burden have independent prognostic significance for patients with lower risk myelodysplastic syndromes. Leukemia, 27, 2077-2081.

20. Galbiati, S., Brisci, A., Lalatta, F., Seia, M., Makrigiorgos, G. M., Ferrari, M. and Cremonesi, L. (2011) Full COLD-PCR protocol for noninvasive prenatal diagnosis of genetic diseases. Clin Chem, 57, 136-138.

### Example 3. Use of Oligonucleotides to Make dsDNA from ssDNA

FIGS. 24A-C provide an example of using oligonucleotides (e.g., F1 and R1 with sequences SEQ ID NOs: 1 and 2, respectively) to make probes using a single strand sequence of NOP14 (SEQ ID NO: 37). The BST polymerase makes ds NOP14 sequences by extending the oligonucleotides F1 and R1. The resultant dsDNA sequences are nicked by the DSN for subsequent formation of dsDNA.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method of generating probes from a sample of input double-stranded DNA (dsDNA), the method comprising:
(a) forming a reaction mixture comprising:
the input dsDNA,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and
deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and
(b) subjecting the reaction mixture to the temperature T under which both the nicking nuclease and the strand-displacing polymerase are active, thereby forming the probes.

2. The method of claim 1, further comprising, prior to forming the reaction mixture, forming the input dsDNA from input ssDNA.

3. The method of claim 2, wherein the forming of input dsDNA comprises:
forming a reaction mixture comprising;
the input ssDNA,
deoxynucleotidyl transferase (TdT), wherein the TdT has the ability to add a polyA tail to the 3′ end of ssDNA, and
poly-dT-primers, wherein the poly-dT primers consist of equal amounts of a poly-dT-primer with an extra G nucleotide at the 3′ end, a poly-dT-primer with an extra C nucleotide at the 3′ end, and a poly-dT-primer with an extra A nucleotide at the 3′ end;
forming a polyA tail on the 3′ end of the ssDNA;
subjecting the reaction mixture to a temperature under which the poly-dT-primers anneal to the polyA tails on the ssDNA; and
subjecting the reaction mixture to a polymerase and to a temperature under which the poly-dT-primers extend to form dsDNA.

4. The method of claim 2, wherein forming the input dsDNA comprises performing a Klenow-fragment enzymatic reaction on the input ssDNA in the presence of random oligonucleotides.

5. The method of claim 2, wherein forming the input dsDNA comprises:
forming a reaction mixture comprising the input ssDNA, one or more oligonucleotides that are complementary to part of the input ssDNA; and
subjecting the reaction mixture to a temperature and a polymerase under which the oligonucleotides extend to form dsDNA.

6. The method of claim 1, further comprising synthesizing the input dsDNA as complementary DNA (cDNA) from RNA.

7. A method of generating probes from a sample of input single-stranded DNA (ssDNA), the method comprising:
(a) forming a reaction mixture comprising:
the input ssDNA,
one or more oligonucleotides that are complementary to at least a part of the input ssDNA, wherein the oligonucleotides are capable of extending in the presence of a strand-displacing polymerase to form dsDNA,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the input ssDNA and/or single strand of dsDNA having the break and displaces the ssDNA fragment that is 3′ relative to the break, and
deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and
(b) subjecting the reaction mixture to the temperature T under which both the nicking nuclease and the strand-displacing polymerase are active, thereby forming the probes.

8. The method of any one of the preceding claims, wherein the generated probes are a collection of dsDNA, wherein each dsDNA is a portion of the input dsDNA having corresponding or shared sequence with the input dsDNA, and wherein the collection of dsDNA randomly provides a coverage of 100×-10,000× of the input dsDNA.

9. The method of any one of the preceding claims, wherein the average length of the generated probes is 15-70 bp.

10. The method of any one of the preceding claims, wherein the amount in nanograms of generated probes is 100-10,000 times higher than the amount of input dsDNA.

11. The method of any one of the preceding claims, wherein the reaction mixture is subjected to a temperature T for a time period of 4-5 minutes.

12. The method of any one of the preceding claims, further comprising inactivating the nicking nuclease and the strand-displacing polymerase.

13. The method of any one of the preceding claims, further comprising separating the nicking nuclease and the strand-displacing polymerase from the reaction mixture.

14. The method of any one of the preceding claims, further comprising separating the generated probes from the reaction mixture.

15. The method of any one of the preceding claims, wherein the nicking nuclease is selected from the group consisting of: double-stranded DNA specific nuclease (DSN), Shrimp-based double strand specific nucleases (dsDNase), HL-dsDNAse, and DNAse I.

16. The method of any one of the preceding claims, wherein the strand-displacing polymerase is selected from the group consisting of: a Bst DNA polymerase, phi29 polymerase, and Klenow fragment of DNA polymerase I.

17. The method of claim 15 or 16, wherein the nicking nuclease is selected from the group consisting of: double-stranded DNA specific nuclease (DSN), Shrimp-based double strand specific nucleases (dsDNase), and HL-dsDNAse and at a concentration of 0.1-0.3 units; and the strand-displacing polymerase is a Bst DNA polymerase and at a concentration of 6-10 units; and the temperature T is 30-70° C.

18. The method of claim 15 or 16, wherein the nicking nuclease is selected from the group consisting of: double-stranded DNA specific nuclease (DSN), Shrimp-based double strand specific nucleases (dsDNase), and HL-dsDNAse and at a concentration of 0.1-0.3 units; and the strand-displacing polymerase is a phi29 polymerase and at a concentration of 0.1-5 units; and the temperature T is 20-50° C.

19. The method of claim 15 or 16, wherein the nicking nuclease is DNAse I, and the strand-displacing polymerase is Klenow fragment of DNA polymerase I; and the temperature T is 25-45° C.

20. A method of any one of the preceding claims, wherein the dNTPs comprise biotin-dUTP, 2,6 di-amino-purinetriphosphate, and/or d-iosinetriphosphate.

21. The method of any one of the preceding claims, further comprising validating the generated probes, wherein validating the generated probes comprises:
attaching the probes to a solid surface;
incubating the probes with a sample of target DNA fragments to allow hybridization of the probes and target DNA sequences, wherein each target DNA fragment is ligated to an adapter;
washing away unbound DNA fragments;
releasing the target DNA fragments that are hybridized to the probes;
amplifying the released target DNA fragments using primers that are complementary to the adapters;
amplifying the released target DNA fragments using target-specific primers; and
sequencing the amplified released target DNA fragments to determine whether the amplified released target DNA fragments are specific to the probes.

22. A method of interrogating target DNA regions in a sample of DNA, the method comprising:
(a) providing one or more input probes, wherein each input probe is a dsDNA, each single strand of which is complementary to a target DNA region, wherein the target DNA region for each input probe is different from the target DNA region for all other input probes;
(b) generating probes comprising
(i) forming a reaction mixture comprising:
the input probe,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and
deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and
(ii) subjecting the reaction mixture to the temperature T under which both the nicking nuclease and the strand-displacing polymerase are active, thereby forming the probes;
(c) incubating a first aliquot of the generated probes with a sample of DNA suspected to have one or more of the target DNA regions to allow the generated probes to capture complementary target DNA regions;
(d) releasing the captured complementary target DNA regions from the generated probes;
(e) amplifying the released target DNA regions; and
(e) performing sequencing, real time PCR, or other downstream assays on the captured target DNA sequences.

23. A method of interrogating target DNA regions in a sample of DNA, the method comprising:
(a) providing one or more input probes, wherein each input probe is a ssDNA, each single strand of which is complementary to a target DNA region, wherein the target DNA region for each input probe is different from the target DNA region for all other input probes;
(b) generating probes comprising
(i) forming a reaction mixture comprising:
the ss-input probe,
one or more oligonucleotides that are complementary to at least a part of the ss-input probe, wherein the oligonucleotides are capable of extending in the presence of a strand-displacing polymerase to form dsDNA,
a nicking nuclease active at a temperature T, wherein the nicking nuclease incorporates random single-stranded breaks into dsDNA,
a strand-displacing polymerase active at the temperature T, wherein the strand-displacing polymerase recognizes a single-stranded break in dsDNA, and, in the presence of nucleotide triphosphates, extends the single strand having the break and displaces the ssDNA fragment that is 3′ relative to the break, and
deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP); and
(ii) subjecting the reaction mixture to the temperature T under which both the nicking nuclease and the strand-displacing polymerase are active, thereby forming the probes;
(c) incubating a first aliquot of the generated probes with a sample of DNA suspected to have one or more of the target DNA regions to allow the generated probes to capture complementary target DNA regions;
(d) releasing the captured complementary target DNA regions from the generated probes;
(e) amplifying the released target DNA regions; and
(e) performing sequencing, real time PCR, or other downstream assays on the captured target DNA sequences.

24. The method of claim 22 or 23, further comprising forming the input dsDNA probes from input ssDNA probes.

25. The method of claim 24, wherein the forming of input dsDNA probes comprises:
forming a reaction mixture comprising;
one or more input ssDNA probes,
deoxynucleotidyl transferase (TdT), wherein the TdT has the ability to add a polyA tail to the 3′ end of ssDNA,
poly-dT-primers, wherein the poly-dT primers consist of equal amounts of a poly-dT-primer with an extra G nucleotide at the 3′ end, a poly-dT-primer with an extra C nucleotide at the 3′ end, and a poly-dT-primer with an extra A nucleotide at the 3′ end; and
forming a polyA tail on the 3′ end of the ssDNA probes;
permitting annealing of the poly-dT-primers to the polyA tails on the ssDNA probes; and
extending the poly-dT-primers to form dsDNA probes.

26. The method of any one of claims 22-25, further comprising, prior to performing sequencing:
incubating the amplified capture target DNA regions with a second aliquot of the generated probes, wherein the second aliquot of generated probes is the same or different from the first aliquot of generated probes;
releasing captured complementary target DNA regions from probes of the second aliquot of generated probes; and
amplifying the released target DNA regions of the second aliquot of generated probes.

27. The method of any one of claims 22-26, wherein the sample of DNA comprises genomic DNA obtained from a biological sample.

28. The method of claims 27, wherein the sample of DNA comprises genomic DNA is that of a micro-organism and the method is used to identify the species of the micro-organism.

29. The method of any one of claims 27, wherein the genomic DNA is from a subject suspected of having one or more mutations in one or more target regions.

30. The method of any one of claims 27-29, further comprising performing end repair to each of the generated probes to form blunt ends, and ligating the repaired ends of the generated probes to sequencing primers.

31. The method of any one of claims 27-30, wherein the biological sample is blood, serum, plasma, urine, cheek swab, a tissue biopsy, a bronchial lavage, or pulmonary brushing.

32. The method of any one of the preceding methods, further comprising forming droplets of the reaction mixture prior to subjecting the reaction mixture to a temperature T.

33. A composition comprising a collection of dsDNA probes, wherein each probe has a sequence that corresponds to a portion of input dsDNA, wherein the collection of probes randomly provides a coverage of 100×-1000× of the input dsDNA.

34. The composition of claim 33, wherein the average length of each probe in the collection of probes is 15-70 bp.

35. A reaction mixture comprising:
a sample of dsDNA,
a nicking nuclease, wherein the nicking nuclease at a temperature T incorporates random single-stranded breaks into dsDNA,
a strand-displacing polymerase, wherein the strand-displacing polymerase at the temperature T recognizes a single-stranded break in dsDNA and in the presence of nucleotide triphosphates extends the single strand having the break and displaces the ssDNA fragment that is 3′ relative to the break, and
deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP).

36. A reaction mixture comprising:
a sample of ssDNA,
one or more oligonucleotides that are complementary to at least a part of the input ssDNA, wherein the oligonucleotides are capable of extending in the presence of a strand-displacing polymerase to form dsDNA,
a nicking nuclease, wherein the nicking nuclease at a temperature T incorporates random single-stranded breaks into dsDNA,
a strand-displacing polymerase, wherein the strand-displacing polymerase at the temperature T recognizes a single-stranded break in dsDNA and in the presence of nucleotide triphosphates extends the single strand having the break and displaces the ssDNA fragment that is 3′ relative to the break, and
deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP).

37. The reaction mixture of claim 35 or 36, wherein the nicking nuclease is selected from the group consisting of: double-stranded DNA specific nuclease (DSN), Shrimp-based double strand specific nucleases (dsDNase), HL-dsDNAse, and DNAse I.

38. The reaction mixture of any one of claims 35-37, wherein the strand-displacing polymerase is selected from the group consisting of: Bst DNA polymerase, phi29 polymerase, and Klenow fragment of DNA polymerase I.

39. The reaction mixture of any one of claims 35-38, further comprising Mg²⁺.

40. The reaction mixture of any one of the claims 35-39, wherein the dNTPs comprise dATP, dGTP, dCTP, dTTP, or analogs thereof.

41. The reaction mixture of any one of claims 35-40, wherein the nicking nuclease is selected from the group consisting of: double-stranded DNA specific nuclease (DSN), Shrimp-based double strand specific nucleases (dsDNase), and HL-dsDNAse; and the strand-displacing polymerase is a Bst DNA polymerase, and wherein the reaction mixture is at a temperature of 44-56° C.

42. The reaction mixture of any one of claims 35-41, wherein the nicking nuclease is DNAse I, and the strand-displacing polymerase is Klenow fragment of DNA polymerase I, and wherein the reaction mixture is at a temperature of 34-40° C.

43. A kit comprising:
a nicking nuclease, wherein the nicking nuclease at a temperature T incorporates random single-stranded breaks into dsDNA,
a strand-displacing polymerase, wherein the strand-displacing polymerase at the temperature T recognizes a single-stranded break in dsDNA and in the presence of nucleotide triphosphates extends the single strand having the break and displaces the ssDNA fragment that is 3′ relative to the break, and
deoxynucleotide triphosphates (dNTPs), wherein the dNTPs comprise one or more the following dNTPs: deoxyadenosine triphosphate (dATP), deoxythymidine triphosphate (dTTP); deoxycytosine triphosphate (dCTP), and deoxyguanosine triphosphate (dGTP).

44. The kit of claim 43, further comprising biotin-dUTP, 2,6 di-amino-purine, and/or d-iosinetriphosphate.


SEQUENCE LISTING (36 sequences)

SEQ ID NO: 1 - DNA, 22 bp, Artificial Sequence
  Synthetic Polynucleotide
gcagatgtgaggtaatgtccag

SEQ ID NO: 2 - DNA, 20 bp, Artificial Sequence
  Synthetic Polynucleotide
tgcggaaagaaagcggaaag

SEQ ID NO: 3 - DNA, 21 bp, Artificial Sequence
  Synthetic Polynucleotide
tgcttctgaaaggcagtcttc

SEQ ID NO: 4 - DNA, 27 bp, Artificial Sequence
  Synthetic Polynucleotide
agcgttaatgatgatgaagataatttg

SEQ ID NO: 5 - DNA, 20 bp, Artificial Sequence
  Synthetic Polynucleotide
ctgtgagagagaacaatggc

SEQ ID NO: 6 - DNA, 23 bp, Artificial Sequence
  Synthetic Polynucleotide
agccattcacataaatgactcct

SEQ ID NO: 7 - DNA, 21 bp, Artificial Sequence
  Synthetic Polynucleotide
ggtctgggttccagaaatgag

SEQ ID NO: 8 - DNA, 21 bp, Artificial Sequence
  Synthetic Polynucleotide
agcatctcagtgattgacagg

SEQ ID NO: 9 - DNA, 20 bp, Artificial Sequence
  Synthetic Polynucleotide
gacggaagctcaagctcttc

SEQ ID NO: 10 - DNA, 21 bp, Artificial Sequence
  Synthetic Polynucleotide
cccatctccgtaagaaaagca

SEQ ID NO: 11 - DNA, 22 bp, Artificial Sequence
  Synthetic Polynucleotide
ccattgtgctactgtcagtcat

SEQ ID NO: 12 - DNA, 17 bp, Artificial Sequence
  Synthetic Polynucleotide
cccgttccctgtcccac

SEQ ID NO: 13 - DNA, 24 bp, Artificial Sequence
  Synthetic Polynucleotide
ggatgagtttgtctgaaagatcct

SEQ ID NO: 14 - DNA, 20 bp, Artificial Sequence
  Synthetic Polynucleotide
aggggaggaaaaaggtcttc

SEQ ID NO: 15 - DNA, 26 bp, Artificial Sequence
  Synthetic Polynucleotide
tgtcttttcctttctctgttttctct

SEQ ID NO: 16 - DNA, 22 bp, Artificial Sequence
  Synthetic Polynucleotide
actgtacgaagactggattgtc

SEQ ID NO: 17 - DNA, 19 bp, Artificial Sequence
  Synthetic Polynucleotide
tggctttgcaggtcgaagg

SEQ ID NO: 18 - DNA, 22 bp, Artificial Sequence
  Synthetic Polynucleotide
gtgagctacctgatgatgaacc

SEQ ID NO: 19 - DNA, 20 bp, Artificial Sequence
  Synthetic Polynucleotide
gccacttcgttagaagaccg

SEQ ID NO: 20 - DNA, 23 bp, Artificial Sequence
  Synthetic Polynucleotide
tggtgatagtagtgtgtctgtcc

SEQ ID NO: 21 - DNA, 22 bp, Artificial Sequence
  Synthetic Polynucleotide
gctgtatcagatcgcaatttcc

SEQ ID NO: 22 - DNA, 27 bp, Artificial Sequence
  Synthetic Polynucleotide
catcttttgcttatatttcctggaagt

SEQ ID NO: 23 - DNA, 25 bp, Artificial Sequence
  Synthetic Polynucleotide
atttcagtgttacttacctgtcttg

SEQ ID NO: 24 - DNA, 20 bp, Artificial Sequence
  Synthetic Polynucleotide
acaggctcaggacttagcaa

SEQ ID NO: 25 - DNA, 24 bp, Artificial Sequence
  Synthetic Polynucleotide
accacattacatacttaccatgcc

SEQ ID NO: 26 - DNA, 20 bp, Artificial Sequence
  Synthetic Polynucleotide
tgggcagattacagtgggac

SEQ ID NO: 27 - DNA, 23 bp, Artificial Sequence
  Synthetic Polynucleotide
gaacagctttgaggtgggtgttt

SEQ ID NO: 28 - DNA, 19 bp, Artificial Sequence
  Synthetic Polynucleotide
tggtctcctccaccgcttc

SEQ ID NO: 29 - DNA, 32 bp, Artificial Sequence
  Synthetic Polynucleotide
acactctttccctacacgacctcttccgatct

SEQ ID NO: 30 - DNA, 31 bp, Artificial Sequence
  Synthetic Polynucleotide
gactggagttcagacgtgtctcttccgatct

SEQ ID NO: 31 - DNA, 10 bp, Artificial Sequence
  Synthetic Polynucleotide
tttttcctct

SEQ ID NO: 32 - DNA, 12 bp, Artificial Sequence
  Synthetic Polynucleotide
ctttttcctctt

SEQ ID NO: 33 - DNA, 77 bp, Artificial Sequence
  Synthetic Polynucleotide
agtggcctgacaccccccttcccgtgcaggtgtctgataactctgccgcggtcgtgccggcccagtacatctgtgcc

SEQ ID NO: 34 - DNA, 77 bp, Artificial Sequence
  Synthetic Polynucleotide
attgtactcacatcactgacttgcttcgtgttctgttttgcctggaccataactggggagtccacaatgctggtaaa

SEQ ID NO: 35 - DNA, 62 bp, Artificial Sequence
  Synthetic Polynucleotide
gctgatgctgtgcggcatgcaggagatagacatgagcgactggcagaagagcaccatctacc

SEQ ID NO: 36 - DNA, 81 bp, Artificial Sequence
  Synthetic Polynucleotide
ttggatccacttggcatcccccttggagtgcatcaggcagagaatctcagccaaccagtttgttcctggaaaaagaaggaa